(19) United States
(12) Patent Application Publication
(10) Pub. No.: US 2008/0167309 A1
(43) Pub. Date: Jul. 10, 2008

(54) Pharmaceutical Compounds

(75) Inventors: Valerio Berdini, Cambridge (GB); Theresa Rachael Early, Macclesfield (GB); Michael Alistair O'Brien, Hitchen (GB); Andrew James Woodhead, Cambridge (GB); Paul Graham Wyatt, Perth (GB)

Correspondence Address: HESLIN ROTHENBERG FARLEY & MESITI PC, 5 COLUMBIA CIRCLE, ALBANY, NY, 12203, US

(73) Assignee: ASTEX THERAPEUTICS, LTD., Cambrige (UK)

(21) Appl. No.: 11/572,305

(22) PCT Filed: Jul. 22, 2005
(86) PCT No.: PCT/GB05/02888

§ 371 Date: Dec. 21, 2007

(30) Foreign Application Priority Data
Jul. 22, 2004 (GB) 0416373.9
Jan. 22, 2005 (GB) 0501310.7

(51) Int. Cl. A61K 31/535; A61K 31/445; A61K 31/497; A61K 31/425; A61K 31/44

(52) U.S. Cl. 514/236.8; 514/326; 514/318; 514/254.02; 514/371; 514/253.1; 514/342

(57) ABSTRACT

The invention provides a compound for use in the prophylaxis or treatment of a disease state or condition mediated by a cyclin dependent kinase, the compound having the formula (I):

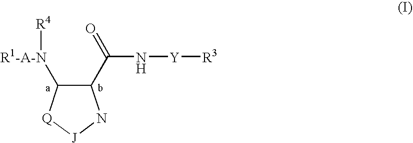

and salts, tautomers, N-oxides or solvates thereof,
wherein
A is a bond, C═O, NR^{g}(C═O) or O(C═O) wherein R^{g} is hydrogen or C₁₋₄ hydrocarbyl optionally substituted by hydroxy or C₁₋₄ alkoxy;
Y is a bond or an alkylene chain of 1, 2 or 3 carbon atoms in length;
Q is S or CR²;
J is S or CH; provided that one of Q and J is S, and the other of Q and J is not S;
   when Q is S, there is a double bond between the ring carbon atoms “a” and “b” and a double bond between the ring nitrogen N and J; and when J is S, there is a double bond between Q and the ring carbon atom “a” and a double bond between the ring nitrogen N and the ring carbon atom “b”;
and R¹ to R⁴ are as defined in the claims.

### CROSS REFERENCE TO RELATED APPLICATIONS

This application is a national phase filing under 35 USC§371 of PCT International Application PCT/GB2005/002888, filed July 22, 2005, and published under PCT Article 21(2) in English as WO 2006/008545 on Jan. 26, 2006. PCT/GB2005/002888 claimed priority from British application 0416373.9, filed Jul. 22, 2004 and British application 0501310.7, filed Jan. 22, 2005. The entire contents of each of the prior applications are incorporated herein by reference.

This invention relates to isothiazole and thiazole compounds that inhibit or modulate the activity of cyclin dependent kinases (CDK) and glycogen synthase kinase-3 (GSK-3), to the use of the compounds in the treatment or prophylaxis of disease states or conditions mediated by cyclin dependent kinases and glycogen synthase kinase-3, and to novel compounds having cyclin dependent kinase or glycogen synthase kinase-3 inhibitory or modulating activity. Also provided are pharmaceutical compositions containing the compounds and novel chemical intermediates.

### BACKGROUND OF THE INVENTION

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, Calif.). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB J, 9:576-596 (1995); Knighton, et al., Science, 253:407-414 (1991); Hiles, et al., Cell, 70:419-429 (1992); Kunz, et al., Cell, 73:585-596 (1993); Garcia-Bustos, et al., EMBO J., 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signalling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signalling pathways to activate or inactivate (either directly or indirectly), for example, a metabolic enzyme, regulatory protein, receptor, cytoskeletal protein, ion channel or pump, or transcription factor. Uncontrolled signalling due to defective control of protein phosphorylation has been implicated in a number of diseases, including, for example, inflammation, cancer, allergy/asthma, disease and conditions of the immune system, disease and conditions of the central nervous system, and angiogenesis.

The process of eukaryotic cell division may be broadly divided into a series of sequential phases termed G1, S, G2 and M. Correct progression through the various phases of the cell cycle has been shown to be critically dependent upon the spatial and temporal regulation of a family of proteins known as cyclin dependent kinases (CDKs) and a diverse set of their cognate protein partners termed cyclins. CDKs are cdc2 (also known as CDK1) homologous serine-threonine kinase proteins that are able to utilise ATP as a substrate in the phosphorylation of diverse polypeptides in a sequence dependent context. Cyclins are a family of proteins characterised by a homology region, containing approximately 100 amino acids, termed the “cyclin box” which is used in binding to, and defining selectivity for, specific CDK partner proteins.

Modulation of the expression levels, degradation rates, and activation levels of various CDKs and cyclins throughout the cell cycle leads to the cyclical formation of a series of CDK/cyclin complexes, in which the CDKs are enzymatically active. The formation of these complexes controls passage through discrete cell cycle checkpoints and thereby enables the process of cell division to continue. Failure to satisfy the pre-requisite biochemical criteria at a given cell cycle checkpoint, i.e. failure to form a required CDK/cyclin complex, can lead to cell cycle arrest and/or cellular apoptosis. Aberrant cellular proliferation, as manifested in cancer, can often be attributed to loss of correct cell cycle control. Inhibition of CDK enzymatic activity therefore provides a means by which abnormally dividing cells can have their division arrested and/or be killed. The diversity of CDKs, and CDK complexes, and their critical roles in mediating the cell cycle, provides a broad spectrum of potential therapeutic targets selected on the basis of a defined biochemical rationale.

Progression from the G1 phase to the S phase of the cell cycle is primarily regulated by CDK2, CDK3, CDK4 and CDK6 via association with members of the D and E type cyclins. The D-type cyclins appear instrumental in enabling passage beyond the G1 restriction point, where as the CDK2/cyclin E complex is key to the transition from the G1 to S phase. Subsequent progression through S phase and entry into G2 is thought to require the CDK2/cyclin A complex. Both mitosis, and the G2 to M phase transition which triggers it, are regulated by complexes of CDK1 and the A and B type cyclins.

During G1 phase Retinoblastoma protein (Rb), and related pocket proteins such as p130, are substrates for CDK(2, 4, & 6)/cyclin complexes. Progression through G1 is in part facilitated by hyperphosphorylation, and thus inactivation, of Rb and p130 by the CDK(4/6)/cyclin-D complexes. Hyperphosphorylation of Rb and p130 causes the release of transcription factors, such as E2F, and thus the expression of genes necessary for progression through G1 and for entry into S-phase, such as the gene for cyclin E. Expression of cyclin E facilitates formation of the CDK2/cyclin E complex which amplifies, or maintains, E2F levels via further phosphorylation of Rb. The CDK2/cyclin E complex also phosphorylates other proteins necessary for DNA replication, such as NPAT, which has been implicated in histone biosynthesis. G1 progression and the G1/S transition are also regulated via the mitogen stimulated Myc pathway, which feeds into the CDK2/cyclin E pathway. CDK2 is also connected to the p53 mediated DNA damage response pathway via p53 regulation of p21 levels. p21 is a protein inhibitor of CDK2/cyclin E and is thus capable of blocking, or delaying, the G1/S transition. The CDK2/cyclin E complex may thus represent a point at which biochemical stimuli from the Rb, Myc and p53 pathways are to some degree integrated. CDK2 and/or the CDK2/cyclin E complex therefore represent good targets for therapeutics designed at arresting, or recovering control of, the cell cycle in aberrantly dividing cells.

The exact role of CDK3 in the cell cycle is not clear. As yet no cognate cyclin partner has been identified, but a dominant negative form of CDK3 delayed cells in G1, thereby suggesting that CDK3 has a role in regulating the G1/S transition.

There is evidence that particular components of the CDK4/cyclin D-INK4 proteins-Rb family regulatory machinery act as tumor suppressors or protooncogenes, whose mutations occur so frequently (>90%) as to suggest that perturbing “the RB pathway” may be involved in the formation of cancer cells. RB loss and mutations inactivating p16^{INK4a} function occurs in many tumor types. Mutually exclusive events resulting in RB or p16^{INK4a} inactivation through mutation, deletion, or epigenetic silencing, or in the overexpression of cyclin D1 or Cdk4, provide genetic evidence for operation of this signaling pathway in tumor surveillance.

Cancers that experience INK4a and RB loss of function, and cyclin D1 or Cdk4 overexpression, include retinoblastomas, small cell lung carcinomas, non-small lung carcinomas, sarcomas, gliomas, pancreatic cancers, head, neck and breast cancers and mantle cell lymphomas in particular small cell lung cancer, non-small cell lung cancer, pancreatic cancer, breast cancer, glioblastoma multiforme, T cell ALL and mantle cell lymphoma.

Although most CDKs have been implicated in regulation of the cell cycle there is evidence that certain members of the CDK family are involved in other biochemical processes. This is exemplified by CDK5 which is necessary for correct neuronal development and which has also been implicated in the phosphorylation of several neuronal proteins such as Tau, NUDE-1, synapsin1, DARPP32 and the Munc18/Syntaxin1A complex. Neuronal CDK5 is conventionally activated by binding to the p35/p39 proteins. CDK5 activity can, however, be deregulated by the binding of p25, a truncated version of p35. Conversion of p35 to p25, and subsequent deregulation of CDK5 activity, can be induced by ischemia, excitotoxicity, and β-amyloid peptide. Consequently p25 has been implicated in the pathogenesis of neurodegenerative diseases, such as Alzheimer's, and is therefore of interest as a target for therapeutics directed against these diseases.

CDK7 is a nuclear protein that has cdc2 CAK activity and binds to cyclin H. CDK7 has been identified as component of the TFIIH transcriptional complex which has RNA polymerase II C-terminal domain (CTD) activity. This has been associated with the regulation of HIV-1 transcription via a Tat-mediated biochemical pathway. CDK8 binds cyclin C and has been implicated in the phosphorylation of the CTD of RNA polymerase II. Similarly the CDK9/cyclin-T1 complex (P-TEFb complex) has been implicated in elongation control of RNA polymerase II. PTEF-b is also required for activation of transcription of the HIV-1 genome by the viral transactivator Tat through its interaction with cyclin T1. CDK7, CDK8, CDK9 and the P-TEFb complex are therefore potential targets for anti-viral therapeutics.

At a molecular level mediation of CDK/cyclin complex activity requires a series of stimulatory and inhibitory phosphorylation, or dephosphorylation, events. CDK phosphorylation is performed by a group of CDK activating kinases (CAKs) and/or kinases such as weel, Myt1 and Mik1. Dephosphorylation is performed by phosphatases such as cdc25(a & c), pp2a, or KAP.

CDK/cyclin complex activity may be further regulated by two families of endogenous cellular proteinaceous inhibitors: the Kip/Cip family, or the INK family. The INK proteins specifically bind CDK4 and CDK6. p16^{ink4} (also known as MTS1) is a potential tumour suppressor gene that is mutated, or deleted, in a large number of primary cancers. The Kip/Cip family contains proteins such as p21^{Cip1,Waf1}, p27^{Kip1} and p57^{Kip2}. As discussed previously p21 is induced by p53 and is able to inactivate the CDK2/cyclin(E/A) and CDK4/cyclin(D1/D2/D3) complexes. Atypically low levels of p27 expression have been observed in breast, colon and prostate cancers. Conversely over expression of cyclin E in solid tumours has been shown to correlate with poor patient prognosis. Over expression of cyclin D1 has been associated with oesophageal, breast, squamous, and non-small cell lung carcinomas.

The pivotal roles of CDKs, and their associated proteins, in co-ordinating and driving the cell cycle in proliferating cells have been outlined above. Some of the biochemical pathways in which CDKs play a key role have also been described. The development of monotherapies for the treatment of proliferative disorders, such as cancers, using therapeutics targeted generically at CDKs, or at specific CDKs, is therefore potentially highly desirable. CDK inhibitors could conceivably also be used to treat other conditions such as viral infections, autoimmune diseases and neuro-degenerative diseases, amongst others. CDK targeted therapeutics may also provide clinical benefits in the treatment of the previously described diseases when used in combination therapy with either existing, or new, therapeutic agents. CDK targeted anticancer therapies could potentially have advantages over many current antitumour agents as they would not directly interact with DNA and should therefore reduce the risk of secondary tumour development.

Glycogen Synthase Kinase-3 (GSK3) is a serine-threonine kinase that occurs as two ubiquitously expressed isoforms in humans (GSK3α & beta GSK3β). GSK3 has been implicated as having roles in embryonic development, protein synthesis, cell proliferation, cell differentiation, microtubule dynamics, cell motility and cellular apoptosis. As such GSK3 has been implicated in the progression of disease states such as diabetes, cancer, Alzheimer's disease, stroke, epilepsy, motor neuron disease and/or head trauma. Phylogenetically GSK3 is most closely related to the cyclin dependent kinases (CDKs).

The consensus peptide substrate sequence recognised by GSK3 is (Ser/Thr)-X-X-X-(pSer/pThr), where X is any amino acid (at positions (n+1), (n+2), (n+3)) and pSer and pThr are phospho-serine and phospho-threonine respectively (n+4). GSK3 phosphorylates the first serine, or threonine, at position (n). Phospho-serine, or phospho-threonine, at the (n+4) position appears necessary for priming GSK3 to give maximal substrate turnover. Phosphorylation of GSK3α at Ser21, or GSK3β at Ser9, leads to inhibition of GSK3. Mutagenesis and peptide competition studies have led to the model that the phosphorylated N-terminus of GSK3 is able to compete with phospho-peptide substrate (S/TXXXpS/pT) via an autoinhibitory mechanism. There are also data suggesting that GSK3α and GSKβ may be subtly regulated by phosphorylation of tyrosines 279 and 216 respectively. Mutation of these residues to a Phe caused a reduction in in vivo kinase activity. The X-ray crystallographic structure of GSK3β has helped to shed light on all aspects of GSK3 activation and regulation.

GSK3 forms part of the mammalian insulin response pathway and is able to phosphorylate, and thereby inactivate, glycogen synthase. Upregulation of glycogen synthase activity, and thereby glycogen synthesis, through inhibition of GSK3, has thus been considered a potential means of combating type II, or non-insulin-dependent diabetes mellitus (NIDDM): a condition in which body tissues become resistant to insulin stimulation. The cellular insulin response in liver, adipose, or muscle tissues, is triggered by insulin binding to an extracellular insulin receptor. This causes the phosphorylation, and subsequent recruitment to the plasma membrane, of the insulin receptor substrate (IRS) proteins. Further phosphorylation of the IRS proteins initiates recruitment of phosphoinositide-3 kinase (PI3K) to the plasma membrane where it is able to liberate the second messenger phosphatidylinosityl 3,4,5-trisphosphate (PIP3). This facilitates co-localisation of 3-phosphoinositide-dedependent protein kinase 1 (PDK1) and protein kinase B (PKB or Akt) to the membrane, where PDK1 activates PKB. PKB is able to phosphorylate, and thereby inhibit, GSK3α and/or GSKβ through phosphorylation of Ser9, or Ser21, respectively. The inhibition of GSK3 then triggers upregulation of glycogen synthase activity. Therapeutic agents able to inhibit GSK3 may thus be able to induce cellular responses akin to those seen on insulin stimulation. A further in vivo substrate of GSK3 is the eukaryotic protein synthesis initiation factor 2B (eIF2B). eIF2B is inactivated via phosphorylation and is thus able to suppress protein biosynthesis. Inhibition of GSK3, e.g. by inactivation of the “mammalian target of rapamycin” protein (mTOR), can thus upregulate protein biosynthesis. Finally there is some evidence for regulation of GSK3 activity via the mitogen activated protein kinase (MAPK) pathway through phosphorylation of GSK3 by kinases such as mitogen activated protein kinase activated protein kinase 1 (MAPKAP-K1 or RSK). These data suggest that GSK3 activity may be modulated by mitogenic, insulin and/or amino acid stimuli.

It has also been shown that GSK3β is a key component in the vertebrate Wnt signalling pathway. This biochemical pathway has been shown to be critical for normal embryonic development and regulates cell proliferation in normal tissues. GSK3 becomes inhibited in response to Wnt stimuli. This can lead to the de-phosphorylation of GSK3 substrates such as Axin, the adenomatous polyposis coli (APC) gene product and β-catenin. Aberrant regulation of the Wnt pathway has been associated with many cancers. Mutations in APC, and/or β-catenin, are common in colorectal cancer and other tumours. β-catenin has also been shown to be of importance in cell adhesion. Thus GSK3 may also modulate cellular adhesion processes to some degree. Apart from the biochemical pathways already described there are also data implicating GSK3 in the regulation of cell division via phosphorylation of cyclin-D1, in the phosphorylation of transcription factors such as c-Jun, CCAAT/enhancer binding protein α (C/EBPα), c-Myc and/or other substrates such as Nuclear Factor of Activated T-cells (NFATc), Heat Shock Factor-1 (HSF-1) and the c-AMP response element binding protein (CREB). GSK3 also appears to play a role, albeit tissue specific, in regulating cellular apoptosis. The role of GSK3 in modulating cellular apoptosis, via a pro-apoptotic mechanism, may be of particular relevance to medical conditions in which neuronal apoptosis can occur. Examples of these are head trauma, stroke, epilepsy, Alzheimer's and motor neuron diseases, progressive supranuclear palsy, corticobasal degeneration, and Pick's disease. In vitro it has been shown that GSK3 is able to hyper-phosphorylate the microtubule associated protein Tau. Hyperphosphorylation of Tau disrupts its normal binding to microtubules and may also lead to the formation of intra-cellular Tau filaments. It is believed that the progressive accumulation of these filaments leads to eventual neuronal dysfunction and degeneration. Inhibition of Tau phosphorylation, through inhibition of GSK3, may thus provide a means of limiting and/or preventing neurodegenerative effects.

### Diffuse Large B-cell Lymphomas (DLBCL)

Cell cycle progression is regulated by the combined action of cyclins, cyclin-dependent kinases (CDKs), and CDK-inhibitors (CDKi), which are negative cell cycle regulators. p27KIP1 is a CDKi key in cell cycle regulation, whose degradation is required for G1/S transition. In spite of the absence of p27KIP1 expression in proliferating lymphocytes, some aggressive B-cell lymphomas have been reported to show an anomalous p27KIP1 staining. An abnormally high expression of p27KIP1 was found in lymphomas of this type. Analysis of the clinical relevance of these findings showed that a high level of p27KIP1 expression in this type of tumour is an adverse prognostic marker, in both univariate and multivariate analysis. These results show that there is abnormal p27KIP1 expression in Diffuse Large B-cell Lymphomas (DLBCL), with adverse clinical significance, suggesting that this anomalous p27KIP1 protein may be rendered non-functional through interaction with other cell cycle regulator proteins. (Br. J. Cancer. 1999 July; 80(9):1427-34. p27KIP1 is abnormally expressed in Diffuse Large B-cell Lymphomas and is associated with an adverse clinical outcome. Saez A, Sanchez E, Sanchez-Beato M, Cruz M A, Chacon I, Munoz E, Camacho F I, Martinez-Montero J C, Mollejo M, Garcia J F, Piris M A. Department of Pathology, Virgen de la Salud Hospital, Toledo, Spain.)

### Chronic Lymphocytic Leukemia

B-Cell chronic lymphocytic leukaemia (CLL) is the most common leukaemia in the Western hemisphere, with approximately 10,000 new cases diagnosed each year (Parker S L, Tong T, Bolden S, Wingo P A: Cancer statistics, 1997. Ca. Cancer. J. Clin. 47:5, (1997)). Relative to other forms of leukaemia, the overall prognosis of CLL is good, with even the most advanced stage patients having a median survival of 3 years.

The addition of fludarabine as initial therapy for symptomatic CLL patients has led to a higher rate of complete responses (27% v 3%) and duration of progression-free survival (33 v 17 months) as compared with previously used alkylator-based therapies. Although attaining a complete clinical response after therapy is the initial step toward improving survival in CLL, the majority of patients either do not attain complete remission or fail to respond to fludarabine. Furthermore, all patients with CLL treated with fludarabine eventually relapse, making its role as a single agent purely palliative (Rai K R, Peterson B, Elias L, Shepherd L, Hines J, Nelson D, Cheson B, Kolitz J, Schiffer C A: A randomized comparison of fludarabine and chlorambucil for patients with previously untreated chronic lymphocytic leukemia. A CALGB SWOG, CTG/NCI-C and ECOG Inter-Group Study. Blood 88:141a, 1996 (abstr 552, suppl 1). Therefore, identifying new agents with novel mechanisms of action that complement fludarabine's cytotoxicity and abrogate the resistance induced by intrinsic CLL drug-resistance factors will be necessary if further advances in the therapy of this disease are to be realized.

The most extensively studied, uniformly predictive factor for poor response to therapy and inferior survival in CLL patients is aberrant p53 function, as characterized by point mutations or chromosome 17p13 deletions. Indeed, virtually no responses to either alkylator or purine analog therapy have been documented in multiple single institution case series for those CLL patients with abnormal p53 function. Introduction of a therapeutic agent that has the ability to overcome the drug resistance associated with p53 mutation in CLL would potentially be a major advance for the treatment of the disease.

Flavopiridol and CYC 202, inhibitors of cyclin-dependent kinases induce in vitro apoptosis of malignant cells from B-cell chronic lymphocytic leukemia (B-CLL). Flavopiridol exposure results in the stimulation of caspase 3 activity and in caspase-dependent cleavage of p27(kip1), a negative regulator of the cell cycle, which is overexpressed in B-CLL (Blood. 1998 Nov. 15; 92(10):3804-16 Flavopiridol induces apoptosis in chronic lymphocytic leukemia cells via activation of caspase-3 without evidence of bcl-2 modulation or dependence on functional p53. Byrd J C, Shinn C, Waselenko J K, Fuchs E J, Lehman T A, Nguyen P L, Flinn I W, Diehl L F, Sausville E, Grever M R).

### PRIOR ART

WO 02/34721 from Du Pont discloses a class of indeno[1,2-c]pyrazol-4-ones as inhibitors of cyclin dependent kinases.

WO 01/81348 from Bristol Myers Squibb describes the use of 5-thio-, sulphinyl- and sulphonylpyrazolo[3,4-b]-pyridines as cyclin dependent kinase inhibitors.

WO 00/62778 also from Bristol Myers Squibb discloses a class of protein tyrosine kinase inhibitors.

WO 01/72745A1 from Cyclacel describes 2-substituted 4-heteroaryl-pyrimidines and their preparation, pharmaceutical compositions containing them and their use as inhibitors of cyclin-dependant kinases (CDKs) and hence their use in the treatment of proliferative disorders such as cancer, leukaemia, psoriasis and the like.

WO 99/21845 from Agouron describes 4-aminothiazole derivatives for inhibiting cyclin-dependent kinases (CDKs), such as CDK1, CDK2, CDK4, and CDK6. The invention is also directed to the therapeutic or prophylactic use of pharmaceutical compositions containing such compounds and to methods of treating malignancies and other disorders by administering effective amounts of such compounds.

WO 01/53274 from Agouron discloses as CDK kinase inhibitors a class of compounds which can comprise an amide-substituted benzene ring linked to an N-containing heterocyclic group.

WO 01/98290 (Pharmacia & Upjohn) discloses a class of 3-aminocarbonyl-2-carboxamido thiophene derivatives as protein kinase inhibitors.

WO 01/53268 and WO 01/02369 from Agouron disclose compounds that mediate or inhibit cell proliferation through the inhibition of protein kinases such as cyclin dependent kinase or tyrosine kinase.

WO 00/39108, WO 00/59902, U.S. Pat. No. 6,020,357, WO 99/32454, WO 98/28269 and WO 02/00651 (all to Du Pont) describe heterocyclic compounds that are inhibitors of trypsin-like serine protease enzymes, especially factor Xa and thrombin. The compounds are stated to be useful as anticoagulants or for the prevention of thromboembolic disorders.

US 2002/0091116 (Zhu et al.), WO 01/19798 and WO 01/64642 each disclose diverse groups of heterocyclic compounds as inhibitors of Factor Xa.

U.S. Pat. No. 6,127,382, WO 01/70668, WO 00/68191, WO 00/06169, WO 97/48672, WO 97/19052 and WO 97/19062 (all to Allergan) each describe compounds having retinoid-like activity for use in the treatment of various hyperproliferative diseases including cancers.

WO 02/070510 (Bayer) describes a class of amino-dicarboxylic acid compounds for use in the treatment of cardiovascular diseases.

WO 97/03071 (Knoll AG) discloses a class of heterocyclyl-carboxamide derivatives for use in the treatment of central nervous system disorders.

WO 97/40017 (Novo Nordisk) describes compounds that are modulators of protein tyrosine phosphatases.

WO 01/58869 (Bristol Myers Squibb) discloses cannabinoid receptor modulators that can be used inter alia to treat a variety of diseases. The main use envisaged is the treatment of respiratory diseases, although reference is made to the treatment of cancer.

WO 2004/039795 (Fujisawa) discloses amides containing a 1-substituted pyrazole group as inhibitors of apolipoprotein B secretion. The compounds are stated to be useful in treating such conditions as hyperlipidemia.

WO 2004/000318 (Cellular Genomics) discloses various amino-substituted monocycles as kinase modulators.

WO 03/031440 (Schering) discloses a class of maleimide compounds as CXC-chemokine antagonists.

U.S. Pat. No. 5,502,068 (Synphar) discloses cyclopropylpyrroloindole-oligopeptide compounds as anti-cancer agents.

WO 03/040147 (Pharmacia & Upjohn) discloses azabicyclic-substituted heteroaryl compounds and their therapeutic uses.

WO 03/014137 (CV Therapeutics) discloses a class of adenosine receptor antagonists.

WO 02/083624 (Schering) discloses substituted cyclobutene-1,2-diones as CXC-chemokine receptor ligands.

WO 02/064586 (Vertex) discloses amino-substituted heterocycles are ERK2 inhibitors.

WO 02/22601, WO 02/22603, WO 02/22605 and WO 02/22608 (each to Vertex) discloses pyrazole compounds as protein kinase inhibitors.

WO 01/32626 (SmithKline Beecham) discloses isoquinoline and quinazoline compounds having 5HT receptor activity.

WO 98/08845 (Novartis) discloses thiatriazines having herbicidal activity.

WO 2004/087138 (Sucampo AG & Fujisawa) discloses a class of 2-acylamino thiazole compounds for treating vascular hyperpermeable disease.

WO 2004/067521 (Fujisawa) discloses a class of thiazole compounds for treating a VAP-1 associated disease such as macular edema.

WO 03/062392 (Ceretek) discloses methods of treating conditions associated with an EDG receptor.

JP 2000/86641 (Kyorin) discloses a class of substituted benzothiazole compounds.

WO 03/024448 (Methylgene) discloses a class of triazine compounds as histone deacetylase inhibitors.

WO 02/094791, WO 01/70671 and WO 02/070483 (each to Dupont) disclose heterocyclic diamides as invertebrate pest control agents.

WO 99/46244 (Novo Nordisk) discloses a class of heterocyclic amides as inhibitors of protein tyrosine phosphatases such as PTP1B.

WO 02/00649 (AstraZeneca) discloses substituted quinazoline derivatives as aurora kinase inhibitors.

WO 01/52847, WO 02/053156, WO 02/053158, WO 02/07725, WO 02/08210, WO 02/053161 (each to Alteon) discloses thiazole, imidazole and oxazole compounds for various therapeutic uses.

WO 02/088119 (Genesoft) discloses a class of thienyl compounds as antibiotic agents.

WO 2004/012736 (Genesoft) discloses biaryl compounds having anti-infective activity.

WO 02/066470, WO 2004/007458, WO 02/068406 and WO 2004/007481 (each to Amgen) disclose substituted alkylamine derivatives for use in treating cancer and angiogenesis mediated disorders.

WO 2004/089415 and WO 2004/089416 (both to Novo Nordisk) disclose combination therapies involving 11β-hydroxysteroid dehydrogenase type 1 inhibitors.

US 2004/0082629 (Nippon Soda) discloses a class of aminothiazole compounds as insecticidal agents.

### SUMMARY OF THE INVENTION

The invention provides compounds that have cyclin dependent kinase inhibiting or modulating activity, and which it is envisaged will be useful in preventing or treating disease states or conditions mediated by the kinases.

Thus, for example, it is envisaged that the compounds of the invention will be useful in alleviating or reducing the incidence of cancer.

Accordingly, in a first aspect, the invention provides a compound for use in the prophylaxis or treatment of a disease state or condition mediated by a cyclin dependent kinase, the compound having the formula (I):

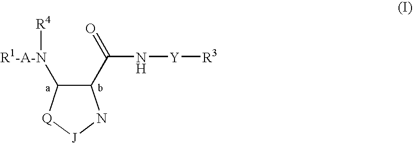

and salts, tautomers, N-oxides or solvates thereof;
wherein

A is a bond, C═O, NR^{g}(C═O) or O(C═O) wherein R^{g} is hydrogen or C₁₋₄ hydrocarbyl optionally substituted by hydroxy or C₁₋₄ alkoxy;

Y is a bond or an alkylene chain of 1, 2 or 3 carbon atoms in length;

Q is S or CR²;

J is S or CH; provided that one of Q and 3 is S, and the other of Q and J is not S;

when Q is S, there is a double bond between the ring carbon atoms “a” and “b” and a double bond between the ring nitrogen N and 3; and when 3 is S, there is a double bond between Q and the ring carbon atom “a” and a double bond between the ring nitrogen N and the ring carbon atom “b”;

R¹ is hydrogen; a carbocyclic or heterocyclic group having from 3 to 12 ring members; or a C₁₋₈ hydrocarbyl group optionally substituted by one or more substituents selected from halogen (e.g. fluorine), hydroxy, C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂;

R² is hydrogen; halogen; C₁₋₄ alkoxy (e.g. methoxy); or a C₁₋₄ hydrocarbyl group optionally substituted by halogen (e.g. fluorine), hydroxyl or C₁₋₄ alkoxy (e.g. methoxy);

R³ is selected from hydrogen and carbocyclic and heterocyclic groups having from 3 to 12 ring members; and

R⁴ is hydrogen or a C₁₋₄ hydrocarbyl group optionally substituted by halogen (e.g. fluorine), hydroxyl or C₁₋₄ alkoxy (e.g. methoxy).

In another aspect, the invention provides a compound of the formula (Ia):

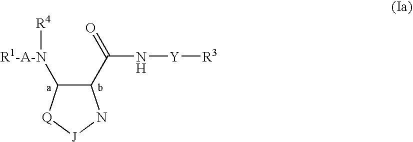

and salts, tautomers, N-oxides and solvates thereof;
wherein

A is a bond, C═O, NR^{g}(C═O) or O(C═O) wherein R^{g} is hydrogen or C₁₋₄ hydrocarbyl optionally substituted by hydroxy or C₁₋₄ alkoxy;

Y is a bond or an alkylene chain of 1, 2 or 3 carbon atoms in length;

Q is S or CR²;

J is S or CH; provided that one of Q and J is S, and the other of Q and J is not S;

when Q is S, there is a double bond between the ring carbon atoms “a” and “b” and a double bond between the ring nitrogen N and J; and when J is S, there is a double bond between Q and the ring carbon atom “a” and a double bond between the ring nitrogen N and the ring carbon atom “b”;

R¹ is (i) a carbocyclic or heterocyclic group having from 3 to 12 ring members; or (ii) a C₁₋₄ hydrocarbyl group substituted by a carbocyclic or heterocyclic group having from 3 to 12 ring members, the hydrocarbyl group being optionally further substituted by one or more substituents selected from halogen (e.g. fluorine), hydroxy, C₁₋₄ hydrocarbyloxy, amino, mono- or di-C₁₋₄ hydrocarbylamino, and wherein 1 or 2 of the carbon atoms of the hydrocarbyl group may optionally be replaced by an atom or group selected from O, S, NH, SO and SO₂;

R² is hydrogen; halogen; C₁₋₄ alkoxy; or a C₁₋₄ hydrocarbyl group optionally substituted by halogen, hydroxy or C₁₋₄ alkoxy;

R³ is selected from carbocyclic and heterocyclic groups having from 3 to 12 ring members; and

R⁴ is hydrogen or a C₁₋₄ hydrocarbyl group optionally substituted by halogen, hydroxy or C₁₋₄ alkoxy;

but excluding:

(A) the compound wherein the ring containing the moiety Q-J is a thiazole ring, A is a bond, R⁴ is hydrogen, R¹ is cyclohexyl, Y is a bond and R³ is a methoxy-substituted dibenzofuran group; and

(B) a compound wherein the ring containing the moiety Q-J is a thiazole ring, A is a bond, R⁴ is hydrogen and R¹ is 4-pyridylmethyl or 5-quinolinyl, and Y—R³ is selected from 3,4-dichlorophenyl, 4-phenoxyphenyl, 4-biphenyl, 4-cyclohexylphenyl and 3-isoquinolinyl.

In one embodiment, the invention provides a compound for use in the prophylaxis or treatment of a disease state or condition mediated by a cyclin dependent kinase, the compound having the formula (Ib):

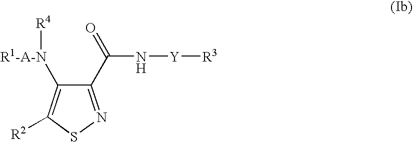

and salts, tautomers, N-oxides or solvates thereof;
wherein

A is a bond, C═O, NR^{g}(C═O) or O(C═O) wherein R^{g} is hydrogen or C₁₋₄ hydrocarbyl optionally substituted by hydroxy or C₁₋₄ alkoxy;

Y is a bond or an alkylene chain of 1, 2 or 3 carbon atoms in length;

R¹ is hydrogen; a carbocyclic or heterocyclic group having from 3 to 12 ring members; or a C₁₋₈ hydrocarbyl group optionally substituted by one or more substituents selected from halogen (e.g. fluorine), hydroxy, C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂;

R² is hydrogen; halogen; C₁₋₄ alkoxy (e.g. methoxy); or a C₁₋₄ hydrocarbyl group optionally substituted by halogen (e.g. fluorine), hydroxyl or C₁₋₄ alkoxy (e.g. methoxy);

R³ is selected from hydrogen and carbocyclic and heterocyclic groups having from 3 to 12 ring members; and

R⁴ is hydrogen or a C₁₋₄ hydrocarbyl group optionally substituted by halogen (e.g. fluorine), hydroxyl or C₁₋₄ alkoxy (e.g. methoxy).

In another embodiment, the invention provides a compound of the formula (Ic):

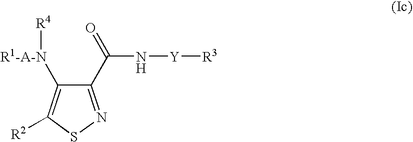

and salts, tautomers, N-oxides and solvates thereof;
wherein

A is a bond, C═O, NR^{g}(C═O) or O(C═O) wherein R^{g} is hydrogen or C₁₋₄ hydrocarbyl optionally substituted by hydroxy or C₁₋₄ alkoxy;

Y is a bond or an alkylene chain of 1, 2 or 3 carbon atoms in length;

R¹ is (i) a carbocyclic or heterocyclic group having from 3 to 12 ring members; or (ii) a C₁₋₄ hydrocarbyl group substituted by a carbocyclic or heterocyclic group having from 3 to 12 ring members, the hydrocarbyl group being optionally further substituted by one or more substituents selected from halogen (e.g. fluorine), hydroxy, C₁₋₄ hydrocarbyloxy, amino, mono- or di-C₁₋₄ hydrocarbylamino, and wherein 1 or 2 of the carbon atoms of the hydrocarbyl group may optionally be replaced by an atom or group selected from O, S, NH, SO and SO₂;

R² is hydrogen; halogen; C₁₋₄ alkoxy; or a C₁₋₄ hydrocarbyl group optionally substituted by halogen, hydroxy or C₁₋₄ alkoxy;

R³ is selected from carbocyclic and heterocyclic groups having from 3 to 12 ring members; and

R⁴ is hydrogen or a C₁₋₄ hydrocarbyl group optionally substituted by halogen, hydroxy or C₁₋₄ alkoxy.

In a further embodiment, the invention provides a compound for use in the prophylaxis or treatment of a disease state or condition mediated by a cyclin dependent kinase, the compound having the formula (Id):

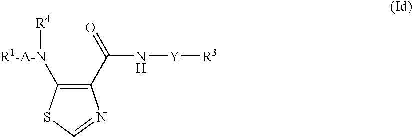

and salts, tautomers, N-oxides or solvates thereof;
wherein

A is a bond, C═O, NR^{g}(C═O) or O(C═O) wherein R^{g} is hydrogen or C₁₋₄ hydrocarbyl optionally substituted by hydroxy or C₁₋₄ alkoxy;

Y is a bond or an alkylene chain of 1, 2 or 3 carbon atoms in length;

R¹ is hydrogen; a carbocyclic or heterocyclic group having from 3 to 12 ring members; or a C₁₋₈ hydrocarbyl group optionally substituted by one or more substituents selected from halogen (e.g. fluorine), hydroxy, C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂;

R³ is selected from hydrogen and carbocyclic and heterocyclic groups having from 3 to 12 ring members; and

R⁴ is hydrogen or a C₁₋₄ hydrocarbyl group optionally substituted by halogen (e.g. fluorine), hydroxyl or C₁₋₄ alkoxy (e.g. methoxy).

In another embodiment, the invention provides a compound of the formula (Ie):

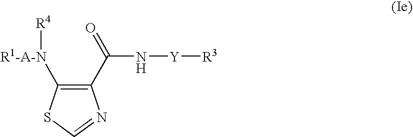

and salts, tautomers, N-oxides and solvates thereof;
wherein

A is a bond, C═O, NR^{g}(C═O) or O(C═O) wherein R^{g} is hydrogen or C₁₋₄ hydrocarbyl optionally substituted by hydroxy or C₁₋₄ alkoxy;

Y is a bond or an alkylene chain of 1, 2 or 3 carbon atoms in length;

R¹ is (i) a carbocyclic or heterocyclic group having from 3 to 12 ring members; or (ii) a C₁₋₄ hydrocarbyl group substituted by a carbocyclic or heterocyclic group having from 3 to 12 ring members, the hydrocarbyl group being optionally further substituted by one or more substituents selected from halogen (e.g. fluorine), hydroxy, C₁₋₄ hydrocarbyloxy, amino, mono- or di-C₁₋₄ hydrocarbylamino, and wherein 1 or 2 of the carbon atoms of the hydrocarbyl group may optionally be replaced by an atom or group selected from O, S, NH, SO and SO₂;

R³ is selected from carbocyclic and heterocyclic groups having from 3 to 12 ring members; and

R⁴ is hydrogen or a C₁₋₄ hydrocarbyl group optionally substituted by halogen, hydroxy or C₁₋₄ alkoxy;

but excluding:

(A) the compound wherein A is a bond, R⁴ is hydrogen, R¹ is cyclohexyl, Y is a bond and R³ is a methoxy-substituted dibenzofuran group; and

(B) a compound wherein A is a bond, R⁴ is hydrogen and R is 4-pyridylmethyl or 5-quinolinyl, and Y—R³ is selected from 3,4-dichlorophenyl, 4-phenoxyphenyl, 4-biphenyl, 4-cyclohexylphenyl and 3-isoquinolyl.

Any one or more of the following optional provisos, in any combination, may apply to the compounds of formulae (I) to (Ie) and sub-groups thereof as defined herein:

(a-i) When A is a bond and Y—R³ is an alkyl, cycloalkyl, optionally substituted phenyl or optionally substituted phenylalkyl, then R¹ is other than a substituted or unsubstituted dihydronaphthalene, dihydrochromen, dihydrothiochroman, tetrahydroquinoline or tetrahydrobenzofuranyl group.

(a-ii) R¹-A-NR⁴— and R³ are each other than a moiety containing a maleimide group wherein the maleimide group has nitrogen atoms attached to the 3- and 4-positions thereof.

(a-iii) R¹ is other than a moiety containing a purine nucleoside group.

(a-iv) R¹-A-NR⁴— and R³ are each other than a moiety containing a cyclobutene-1,2-dione group wherein the cyclobutene-1,2-dione group has nitrogen atoms attached to the 3- and 4-positions thereof.

(a-v) R³ is other than a moiety containing a 4-monosubstituted or 4,5-disubstituted 2-pyridyl or 2-pyrimidinyl group or a 5-monosubstituted or 5,6-disubstituted 1,2,4-triazin-3-yl or 3-pyridazinyl group.

(a-vi) R¹-A-NR⁴— and R³ are each other than a moiety containing a substituted or unsubstituted pyrazol-3-ylamine group linked to a substituted or unsubstituted pyridine, diazine or triazine group.

(a-vii) When A is C═O and Y—R³ is an alkyl, cycloalkyl, optionally substituted phenyl or optionally substituted phenylalkyl group, then R¹ is other than a substituted or unsubstituted tetrahydronaphthalene, tetrahydroquinolinyl, tetrahydrochromanyl or tetrahydrothiochromanyl group.

(a-viii) When R³ is H and A is a bond, R¹ is other than a moiety containing a bis-aryl, bis-heteroaryl or aryl heteroaryl group.

(a-ix) R³ is other than a moiety containing a 1,2,8,8a-tetrahydro-7-methyl-cyclopropa[c]pyrrolo[3,2,e]indole-4-(5H)-one group.

(a-x) When Y is a bond, R³ is hydrogen, A is CO and R¹ is a substituted phenyl group, each substituent on the phenyl group is other than a group CH₂—P(O)R^{x}R^{y} where R^{x} and R^{y} are each selected from alkoxy and phenyl groups.

(b-i) R³ is other than a bridged azabicyclo group.

(b-ii) When A is a bond, then R³ is other than a moiety containing an unsubstituted or substituted phenyl group having attached to an ortho position thereof, a substituted or unsubstituted carbamoyl or thiocarbamoyl group.

(b-iii) When A is a bond, then R³ is other than a moiety containing an isoquinoline or quinoxaline group each having attached thereto a substituted or unsubstituted piperidine or piperazine ring.

(b-iv) When A is a bond and R¹ is an alkyl group, then R³ is other than a moiety containing a thiatriazine group.

(b-v) When R¹ or R³ contain a moiety in which a heterocyclic ring having an S(—O)₂ ring member is fused to a carbocyclic ring, the said carbocyclic ring is other than a substituted or unsubstituted benzene ring

(b-vi) When A is a bond, R¹ is other than an arylalkyl, heteroarylalkyl or piperidinylalkyl group each having attached thereto a substituent selected from cyano, and substituted or unsubstituted amino, aminoalkyl, amidine, guanidine, and carbamoyl groups.

(b-vii) When A is a bond and R¹ is a non-aromatic group, then R³ is other than a six membered monocyclic aryl or heteroaryl group linked directly to a 5,6-fused bicyclic heteroaryl group.

(c-i) When A is a bond, R¹ is other than a substituted arylalkyl, heteroarylalkyl or piperidinylalkyl group.

(c-ii) When R¹-A-NR⁴— is an amino or alkylamino group and Y is a bond, R³ is other than a disubstituted thiazolyl group wherein one of the substituents is selected from cyano and fluoroalkyl.

(c-iii) When A is CO and Y—R³ is (i) a bicyclic heteroaryl group selected from phthalazine, isoquinoline, quinazoline, benzisoxazole and indazole, each of which bicyclic heteroaryl groups bears an amino substituent, or (ii) a phenyl or pyridyl group substituted by one or more substituents selected from methoxy, chlorine, fluorine, amino, aminomethyl, amidino and CONH₂, then R¹ is other than phenyl, pyridyl or pyrimidinyl bearing one or more substituents, one of which is selected from an optionally substituted phenyl, pyridyl or imidazole group.

(c-iv) When A is CO and Y—R³ is (i) a bicyclic heteroaryl group selected from phthalazine, isoquinoline, quinazoline, benzisoxazole and indazole, each of which bicyclic heteroaryl groups bears an amino substituent, or (ii) a phenyl or pyridyl group substituted by one or more substituents selected from methoxy, chlorine, fluorine, amino, aminomethyl, amidino and CONH₂, then R¹ is other than piperidine bearing a substituent selected from an optionally substituted phenyl, pyridyl or imidazole group or an isopropyl group.

(c-v) When A is CO and Y—R³ is (i) a bicyclic heteroaryl group bearing an amino substituent or (ii) an optionally substituted phenyl or pyridyl group, then R¹ is other than phenyl, pyridyl, pyrimidinyl or piperidine bearing one or more substituents, one of which is selected from an optionally substituted phenyl, pyridyl or imidazole group or an isopropyl group.

(c-vi) When the ring containing the moiety Q-J is a thiazole and A is C═O or OC═O, then Y—R³ is other than (i) a 2-aminobenzimidazole group or (ii) a phenyl group bearing a substituent which consists of or terminates in a moiety selected from an optionally protected amino group, —N—CH₂, 2-thiazoline, 2-imidazoline and C(═NH)R^{k}, where R^{k} is hydrogen, alkyl, alkylthio, amino, alkylamino or NH—NH₂.

(c-vii) When A is C═O, R¹ is other than a tetrazole group or a five membered unsaturated or aromatic heterocyclic group attached to the group A through a linking carbon atom of the heterocyclic group and containing, in addition to the linking carbon atom, up to one CH ring member, two or three heteroatom ring members selected from S, O, N and NH and one ring member selected from C—OH, O═S═O, C—SH, C═O, and S═O.

(c-viii) When A is a bond, then R¹ is other than a moiety containing an unsubstituted or substituted quinazoline group.

(c-ix) R¹ is other than a moiety consisting of or containing a substituted triazine group.

(c-x) Y—R³ is other than an ortho-amino phenyl group.

(c-xi) R¹ is other than a moiety consisting of or containing a substituted or unsubstituted benzothiazole group.

(c-xii) The compound is other than one wherein the ring containing the moiety Q-J is a thiazole ring, A is a bond, R⁴ is hydrogen, R¹ is cyclohexyl, Y is a bond and R³ is a methoxy-substituted dibenzofuran group.

(c-xiii) When A is CO and R¹ is (i) a bicyclic heteroaryl group selected from phthalazine, isoquinoline, quinazoline, benzisoxazole and indazole, each of which bicyclic heteroaryl groups bears an amino substituent, or (ii) a phenyl or pyridyl group substituted by one or more substituents selected from methoxy, chlorine, fluorine, amino, aminomethyl, amidino and CONH₂, then Y—R³ is other than phenyl, pyridyl or pyrimidinyl bearing one or more substituents, one of which is selected from an optionally substituted phenyl, pyridyl or imidazole group.

(c-xiv) When A is CO and R¹ is (i) a bicyclic heteroaryl group selected from phthalazine, isoquinoline, quinazoline, benzisoxazole and indazole, each of which bicyclic heteroaryl groups bears an amino substituent, or (ii) a phenyl or pyridyl group substituted by one or more substituents selected from methoxy, chlorine, fluorine, amino, aminomethyl, amidino and CONH₂, then Y—R³ is other than piperidine bearing a substituent selected from an optionally substituted phenyl, pyridyl or imidazole group or an isopropyl group.

(c-xv) When A is CO and R¹ is (i) a bicyclic heteroaryl group bearing an amino substituent or (ii) an optionally substituted phenyl or pyridyl group, then Y—R³ is other than phenyl, pyridyl, pyrimidinyl or piperidine bearing one or more substituents, one of which is selected from an optionally substituted phenyl, pyridyl or imidazole group or an isopropyl group.

(c-xvi) When R¹ contains a substituted or unsubstituted thiophene group, Y—R³ is other than a substituted 5-membered heteroaryl ring containing one to three heteroatom ring members selected from N, S and O.

(c-xvii) When the ring containing the moiety Q-J is a thiazole ring, A is a bond, R⁴ is hydrogen and R¹ is 4-pyridylmethyl or 5-quinolinyl, then Y—R³ is other than 3,4-dichlorophenyl, 4-phenoxyphenyl, 4-biphenyl, 4-cyclohexylphenyl and 3-isoquinolyl.

The reference in proviso (a-iii) to a purine nucleoside group refers to substituted and unsubstituted purine groups having attached thereto a monosaccharide group (e.g. a pentose or hexose) or a derivative of a monosaccharide group, for example a deoxy monosaccharide group or a substituted monosaccharide group.

The reference in proviso (b-i) to a bridged azabicyclo group refers to bicycloalkane bridged ring systems in which one of the carbon atoms of the bicycloalkane has been replaced by a nitrogen atom. In bridged ring systems, two rings share more than two atoms, see for example Advanced Organic Chemistry, by Jerry March, 4^{th} Edition, Wiley Interscience, pages 131-133, 1992.

The provisos (a-i) to (a-x), (b-i) to (b-vii), (c-i) to (c-xvii) in formulae (I) to (Ie) above refer to the disclosures in the following prior art documents.

| | |
|---|---|
| (a-i) | US 2003/0166932, U.S. Pat. No. 6,127,382, |
| | U.S. Pat. No. 6,093,838 |
| (a-ii) | WO 03/031440 |
| (a-iii) | WO 03/014137 |
| (a-iv) | WO 021083624 |
| (a-v) | WO 02/064586 |
| (a-vi) | WO 02/22608, WO 02/22605, WO 02/22603 & WO 02/22601 |
| (a-vii) | WO 97/48672, WO 97/19052 |
| (a-viii) | WO 00/06169 |
| (a-ix) | U.S. Pat. No. 5,502,068 |
| (a-x) | JP 07188269 |
| (b-i) | WO 03/040147 |
| (b-ii) | WO 01/70671 |
| (b-iii) | WO 01/32626 |
| (b-iv) | WO 98/08845 |
| (b-v) | WO 00/59902 |
| (b-vi) | U.S. Pat. No. 6,020,357, WO 99/32454 & WO 98/28269 |
| (b-vii) | WO 2004/012736 |
| (c-i) | U.S. Pat. No. 6,020,357, WO 99/32454 & WO 98/28269 |
| (c-ii) | US 2004/0082629 |
| (c-iii) | WO 02/00651 |
| (c-iv) | WO 02/00651 |
| (c-v) | WO 02/00651 |
| (c-vi) | WO 2004/087138 & WO 2004/067521 |
| (c-vii) | WO 99/46244 |
| (c-viii) | WO 02/00649 |
| (c-ix) | WO 03/024448 |
| (c-x) | WO 03/024448 |
| (c-xi) | JP 2000/086641 |
| (c-xii) | WO 2004/89415 & WO 2004/89416 |
| (c-xiii) | WO 02/00651 |
| (c-xiv) | WO 02/00651 |
| (c-xv) | WO 02/00651 |
| (c-xvi) | WO 02/088119 |
| (c-xvii) | WO 02/066470 & WO 2004/007481 & WO 2004/007458 & |
| | WO 02/068406 |

The invention also provides:
A compound of the formula (I), (Ia), (Ib), (Ic), (Id), (Ie), (II), (IIa), (IIb), (III), (IIIa), (IIIb), (IV), (V), (Va), (Vb), (VI), (VIa), (VII) and (VIII) and sub-groups thereof as defined herein for use in medicine.
A compound of the formula (I), (Ia), (Ib), (Ic), (Id), (Ie), (II), (IIa), (IIb), (III), (IIIa), (IIIb), (IV), (V), (Va), (Vb), (VI), (VIa), (VII) and (VIII) and sub-groups thereof as defined herein for use in the prophylaxis or treatment of a disease state or condition mediated by a cyclin dependent kinase (e.g. CDK1 or CDK2 or CDK4 or CDK5).
The use of a compound of the formula (I), (Ia), (Ib), (Ic), (Id), (Ie), (II), (IIa), (IIb), (III), (IIIa), (IIIb), (IV), (V), (Va), (Vb), (VI), (VIa), (VII) and (VIII) and sub-groups thereof as defined herein for the manufacture of a medicament for the prophylaxis or treatment of a disease state or condition mediated by a cyclin dependent kinase (e.g. CDK1 or CDK2 or CDK4 or CDK5).

A method for alleviating or reducing the incidence of a disease or condition comprising or arising from abnormal cell growth in a mammal, which method comprises administering to the mammal a compound of the formula (I), (Ia), (Ib), (Ic), (Id), (Ie), (II), (IIa), (IIb), (III), (IIIa), (IIIb), (IV), (V), (Va), (Vb), (VI), (VIa), (VII) and (VIII) and sub-groups thereof as defined herein in an amount effective in inhibiting abnormal cell growth.
A method for alleviating or reducing the incidence of a disease state or condition mediated by a cyclin dependent kinase (e.g. CDK1 or CDK2 or CDK4 or CDK5) or glycogen synthase kinase-3, which method comprises administering to a subject in need thereof a compound of the formula (I), (Ia), (Ib), (Ic), (Id), (Ie), (II), (IIa), (IIb), (III), (IIIa), (IIIb), (IV), (V), (Va), (Vb), (VI), (VIa), (VII) and (VIII) and sub-groups thereof as defined herein.
A method for the prophylaxis or treatment of a disease state or condition mediated by a cyclin dependent kinase (e.g. CDK1 or CDK2 or CDK4 or CDK5), which method comprises administering to a subject in need thereof a compound of the formula (I), (Ia), (Ib), (Ic), (Id), (Ie), (II), (IIa), (IIb), (III), (IIa), (IIIb), (IV), (V), (Va), (Vb), (VI), (VIa), (VII) and (VIII) and sub-groups thereof as defined herein.
A method for treating a disease or condition comprising or arising from abnormal cell growth in a mammal, which method comprises administering to the mammal a compound of the formula (I), (Ia), (Ib), (Ic), (Id), (Ie), (II), (IIa), (IIb), (III), (IIa), (IIIb), (IV), (V), (Va), (Vb), (VI), (VIa), (VII) and (VIII) and sub-groups thereof as defined herein in an amount effective in inhibiting abnormal cell growth.
A method for treating a disease or condition comprising or arising from abnormal cell growth in a mammal, the method comprising administering to the mammal a compound of the formula (I), (Ia), (Ib), (Ic), (Id), (Ie), (II), (IIa), (IIb), (III), (IIIa), (IIIb), (IV), (V), (Va), (Vb), (VI), (VIa), (VII) and (VIII) and sub-groups thereof as defined herein in an amount effective to inhibit a cyclin dependent kinase (e.g. CDK1 or CDK2 or CDK4 or CDK5).
A method of inhibiting a cyclin dependent kinase (e.g. CDK1 or CDK2 or CDK4 or CDK5), which method comprises contacting the kinase with a kinase-inhibiting compound of the formula (I), (Ia), (Ib), (Ic), (Id), (Ie), (II), (IIa), (IIb), (III), (IIIa), (IIIb), (IV), (V), (Va), (Vb), (VI), (Via), (VII) and (VIII) and sub-groups thereof as defined herein.
A method of modulating a cellular process (for example cell division) by inhibiting the activity of a cyclin dependent kinase (e.g. CDK1 or CDK2 or CDK4 or CDK5) using a compound of the formula (I), (Ia), (Ib), (Ic), (Id), (Ie), (II), (IIa), (IIb), (III), (IIIa), (IIIb), (IV), (V), (Va), (Vb), (VI), (VIa), (VII) and (VIII) and sub-groups thereof as defined herein.

The compounds of the invention are also considered to be inhibitors of glycogen synthase kinase-3 (GSK3) and, accordingly, the invention also provides methods and uses of kinase inhibitors or modulators of the formula (I), (Ia), (Ib), (Ic), (Id), (Ie), (II), (IIa), (IIb), (III), (IIIa), (IIIb), (IV), (V), (Va), (Vb), (VI), (VIa), (VII) and (VIII) and sub-groups thereof as defined herein but wherein the kinase is glycogen synthase kinase-3.

In further aspects, the invention provides:
A pharmaceutical composition comprising a compound of the formula (I), (Ia), (Ib), (Ic), (Id), (Ie), (II), (IIa), (IIb), (III), (IIIa), (IIIb), (IV), (V), (Va), (Vb), (VI), (VIa), (VII) and (VIII) and sub-groups thereof as defined herein and a pharmaceutically acceptable carrier.
The use of a compound of the formula (I), (Ia), (Ib), (Ic), (Id), (Ie), (II), (IIa), (IIb), (III), (IIIa), (IIIb), (IV), (V), (Va), (Vb), (VI), (VIa), (VII) and (VIII) and sub-groups thereof as defined herein, for the manufacture of a medicament for the prophylaxis or treatment of any one of the disease states or conditions disclosed herein.
A method for the treatment or prophylaxis of any one of the disease states or conditions disclosed herein, which method comprises administering to a patient (e.g. a patient in need thereof) a compound (e.g. a therapeutically effective amount) of the formula (I), (Ia), (Ib), (Ic), (Id), (Ie), (II), (IIa), (IIb), (III), (IIIa), (IIIb), (IV), (V), (Va), (Vb), (VI), (VIa), (VII) and (VIII) and sub-groups thereof as defined herein.
A method for alleviating or reducing the incidence of a disease state or condition disclosed herein, which method comprises administering to a patient (e.g. a patient in need thereof) a compound (e.g. a therapeutically effective amount) of the formula (I), (Ia), (Ib), (Ic), (Id), (Ie), (II), (IIa), (IIb), (III), (IIIa), (IIIb), (IV), (V), (Va), (Vb), (VI), (VIa), (VII) and (VIII) and sub-groups thereof as defined herein.
A method for the diagnosis and treatment of a disease state or condition mediated by a cyclin dependent kinase (e.g. CDK1 or CDK2 or CDK4 or CDK5), which method comprises (i) screening a patient to determine whether a disease or condition from which the patient is or may be suffering is one which would be susceptible to treatment with a compound having activity against cyclin dependent kinases; and (ii) where it is indicated that the disease or condition from which the patient is thus susceptible, thereafter administering to the patient a compound of the formula (I), (Ia), (Ib), (Ic), (Id), (Ie), (II), (IIa), (IIb), (III), (IIIa), (IIIb), (IV), (V), (Va), (Vb), (VI), (VIa), (VII) and (VIII) and sub-groups thereof as defined herein.

The use of a compound of the formula (I), (Ia), (Ib), (Ic), (Id), (Ie), (II), (Ia), (IIb), (III), (IIIa), (IIIb), (IV), (V), (Va), (Vb), (VI), (VIa), (VII) and (VIII) and sub-groups thereof as defined herein for the manufacture of a medicament for the treatment or prophylaxis of a disease state or condition in a patient who has been screened and has been determined as suffering from, or being at risk of suffering from, a disease or condition which would be susceptible to treatment with a compound having activity against cyclin dependent kinase (e.g. CDK1 or CDK2 or CDK4 or CDK5).

### General Preferences and Definitions

The following general preferences and definitions shall apply to each of the moieties T, Q, Y, R^{g}, R¹ to R⁴ and any sub-definition, sub-group or embodiment thereof, unless the context indicates otherwise.

In this section, as in all other sections of this application, unless the context indicates otherwise, a reference to a compound of the formula (I) is to be taken as a reference also to each of formulae (Ia), (Ib), (Ic), (Id), (Ie), (II), (IIa), (IIb), (III), (IIIa), (IIIb), (IV), (V), (Va), (Vb), (VI), (VIa), (VII) or (VIII).

Furthermore, a reference to a compound of formula (I), (Ia), (Ib), (Ic), (Id), (Ie), (II), (IIa), (IIb), (III), (IIIa), (IIIb), (IV), (V), (Va), (Vb), (VI), (VIa), (VII) or (VIII) includes all other subgroups as defined herein. The term ‘subgroups’ includes all preferences, examples and particular compounds defined herein.

Moreover, a reference to a compound of formula (I), (Ia), (Ib), (Ic), (Id), (Ie), (II), (IIa), (IIb), (III), (IIIa), (IIIb), (IV), (V), (Va), (Vb), (VI), (VIa), (VII) or (VIII) and sub-groups thereof includes ionic forms, salts, solvates, isomers, tautomers, N-oxides, esters, prodrugs, isotopes and protected forms thereof, as discussed below, more particularly the salts or tautomers or isomers or N-oxides or solvates thereof, and more preferably, the salts or tautomers or N-oxides or solvates thereof.

For convenience, the compounds of the formulae (I), (Ia), (Ib), (Ic), (Id), (Ie), (II), (IIa), (IIb), (III), (IIa), (IIIb), (IV), (V), (Va), (Vb), (VI), (VIa), (VII) or (VIII) and sub-groups thereof may be referred to also herein as “the compounds of the invention”.

References to “carbocyclic” and “heterocyclic” groups as used herein shall, unless the context indicates otherwise, include both aromatic and non-aromatic ring systems. Thus, for example, the term “carbocyclic and heterocyclic groups” includes within its scope aromatic, non-aromatic, unsaturated, partially saturated and fully saturated carbocyclic and heterocyclic ring systems. In general, such groups may be monocyclic or bicyclic and may contain, for example, 3 to 12 ring members, more usually 5 to 10 ring members. Examples of monocyclic groups are groups containing 3, 4, 5, 6, 7, and 8 ring members, more usually 3 to 7, and preferably 5 or 6 ring members. Examples of bicyclic groups are those containing 8, 9, 10, 11 and 12 ring members, and more usually 9 or 10 ring members.

The carbocyclic or heterocyclic groups can be aryl or heteroaryl groups having from 5 to 12 ring members, more usually from 5 to 10 ring members. The term “aryl” as used herein refers to a carbocyclic group having aromatic character and the term “heteroaryl” is used herein to denote a heterocyclic group having aromatic character. The terms “aryl” and “heteroaryl” embrace polycyclic (e.g. bicyclic) ring systems wherein one or more rings are non-aromatic, provided that at least one ring is aromatic. In such polycyclic systems, the group may be attached by the aromatic ring, or by a non-aromatic ring. The aryl or heteroaryl groups can be monocyclic or bicyclic groups and can be unsubstituted or substituted with one or more substituents, for example one or more groups R¹⁰ as defined herein.

The term “non-aromatic group” embraces unsaturated ring systems without aromatic character, partially saturated and fully saturated carbocyclic and heterocyclic ring systems. The terms “unsaturated” and “partially saturated” refer to rings wherein the ring structure(s) contains atoms sharing more than one valence bond i.e. the ring contains at least one multiple bond e.g. a C═C, C—C or N═C bond. The term “fully saturated” refers to rings where there are no multiple bonds between ring atoms. Saturated carbocyclic groups include cycloalkyl groups as defined below. Partially saturated carbocyclic groups include cycloalkenyl groups as defined below, for example cyclopentenyl, cycloheptenyl and cyclooctenyl. A further example of a cycloalkenyl group is cyclohexenyl.

Examples of heteroaryl groups are monocyclic and bicyclic groups containing from five to twelve ring members, and more usually from five to ten ring members. The heteroaryl group can be, for example, a five membered or six membered monocyclic ring or a bicyclic structure formed from fused five and six membered rings or two fused six membered rings or, by way of a further example, two fused five membered rings. Each ring may contain up to about four heteroatoms typically selected from nitrogen, sulphur and oxygen. Typically the heteroaryl ring will contain up to 4 heteroatoms, more typically up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of five membered heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups.

Examples of six membered heteroaryl groups include but are not limited to pyridine, pyrazine, pyridazine, pyrimidine and triazine.

A bicyclic heteroaryl group may be, for example, a group selected from:
a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a 5- or 6-membered ring containing 1, 2 or 3 ring heteroatoms;
e) a pyrazole ring fused to a 5- or 6-membered ring containing 1 or 2 ring heteroatoms;
f) a pyrazine ring fused to a 5- or 6-membered ring containing 1 or 2 ring heteroatoms;
g) an imidazole ring fused to a 5- or 6-membered ring containing 1 or 2 ring heteroatoms;
h) an oxazole ring fused to a 5- or 6-membered ring containing 1 or 2 ring heteroatoms;
i) an isoxazole ring fused to a 5- or 6-membered ring containing 1 or 2 ring heteroatoms;
j) a thiazole ring fused to a 5- or 6-membered ring containing 1 or 2 ring heteroatoms; f
k) an isothiazole ring fused to a 5- or 6-membered ring containing 1 or 2 ring heteroatoms;
l) a thiophene ring fused to a 5- or 6-membered ring containing 1, 2 or 3 ring heteroatoms;
m) a furan ring fused to a 5- or 6-membered ring containing 1, 2 or 3 ring heteroatoms;
n) a cyclohexyl ring fused to a 5- or 6-membered ring containing 1, 2 or 3 ring heteroatoms; and
o) a cyclopentyl ring fused to a 5- or 6-membered ring containing 1, 2 or 3 ring heteroatoms.

One sub-group of bicyclic heteroaryl groups consists of groups a) to e) and g) to o) above.

Particular examples of bicyclic heteroaryl groups containing a five membered ring fused to another five membered ring include but are not limited to imidazothiazole (e.g. imidazo[2,1-b]thiazole) and imidazoimidazole (e.g. imidazo[1,2-a]imidazole).

Particular examples of bicyclic heteroaryl groups containing a six membered ring fused to a five membered ring include but are not limited to benzofuran, benzothiophene, benzimidazole, benzoxazole, isobenzoxazole, benzisoxazole, benzthiazole, benzisothiazole, isobenzofuran, indole, isoindole, indolizine, indoline, isoindoline, purine (e.g., adenine, guanine), indazole, pyrazolopyrimidine (e.g. pyrazolo[1,5-a]pyrimidine), triazolopyrimidine (e.g. [1,2,4]triazolo[1,5-a]pyrimidine), benzodioxole and pyrazolopyridine (e.g. pyrazolo[1,5-a]pyridine) groups.

Particular examples of bicyclic heteroaryl groups containing two fused six membered rings include but are not limited to quinoline, isoquinoline, chroman, thiochroman, chromene, isochromene, chroman, isochroman, benzodioxan, quinolizine, benzoxazine, benzodiazine, pyridopyridine, quinoxaline, quinazoline, cinnoline, phthalazine, naphthyridine and pteridine groups.

Examples of polycyclic aryl and heteroaryl groups containing an aromatic ring and a non-aromatic ring include tetrahydronaphthalene, tetrahydroisoquinoline, tetrahydroquinoline, dihydrobenzthiene, dihydrobenzofuran, 2,3-dihydro-benzo[1,4]dioxine, benzo[1,3]dioxole, 4,5,6,7-tetrahydrobenzofuran, indoline and indane groups.

Examples of carbocyclic aryl groups include phenyl, naphlithyl, indenyl, and tetrahydronaphthyl groups.

Examples of non-aromatic heterocyclic groups include unsubstituted or substituted (by one or more groups R¹⁰) heterocyclic groups having from 3 to 12 ring members, typically 4 to 12 ring members, and more usually from 5 to 10 ring members. Such groups can be monocyclic or bicyclic, for example, and typically have from 1 to 5 heteroatom ring members (more usually 1, 2, 3 or 4 heteroatom ring members) typically selected from nitrogen, oxygen and sulphur.

When sulphur is present, it may, where the nature of the adjacent atoms and groups permits, exist as —S—, —S(O)— or —S(O)₂—.

The heterocylic groups can contain, for example, cyclic ether moieties (e.g. as in tetrahydrofuran and dioxane), cyclic thioether moieties (e.g. as in tetrahydrothiophene and dithiane), cyclic amine moieties (e.g. as in pyrrolidine), cyclic amide moieties (e.g. as in pyrrolidone), cyclic thioamides, a cyclic urea moiety (e.g. as in imidazolidin-2-one, cyclic thioesters, cyclic ester moieties (e.g. as in butyrolactone), cyclic sulphones (e.g. as in sulpholane and sulpholene), cyclic sulphoxides, cyclic sulphonamides and combinations thereof (e.g. morpholine and thiomorpholine and its S-oxide and S,S-dioxide).

Examples of monocyclic non-aromatic heterocyclic groups include 5-, 6- and 7-membered monocyclic heterocyclic groups. Particular examples include morpholine, piperidine (e.g. 1-piperidinyl, 2-piperidinyl, 3-piperidinyl and 4-piperidinyl), pyrrolidine (e.g. 1-pyrrolidinyl, 2-pyrrolidinyl and 3-pyrrolidinyl), pyrrolidone, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazine, and N-alkyl piperazines such as N-methyl piperazine. Further examples include thiomorpholine and its S-oxide and S,S-dioxide (particularly thiomorpholine). Still further examples include azetidine, piperidone, piperazone, and N-alkyl piperidines such as N-methyl piperidine.

One preferred sub-set of non-aromatic heterocyclic groups consists of saturated groups such as azetidine, pyrrolidine, piperidine, morpholine, thiomorpholine, thiomorpholine S,S-dioxide, piperazine, N-alkyl piperazines, and N-alkyl piperidines.

Examples of non-aromatic carbocyclic groups include cycloalkane groups such as cyclohexyl and cyclopentyl, cycloalkenyl groups such as cyclopentenyl, cyclohexenyl, cycloheptenyl and cyclooctenyl, as well as cyclohexadienyl, cyclooctatetraene, tetrahydronaphthenyl and decalinyl.

Preferred non-aromatic carbocyclic groups are monocyclic rings and most preferably saturated monocyclic rings.

Typical examples are three, four, five and six membered saturated carbocyclic rings, e.g. optionally substituted cyclopentyl and cyclohexyl rings.

One sub-set of non-aromatic carbocylic groups includes unsubstituted or substituted (by one or more groups R¹⁰) monocyclic groups and particularly saturated monocyclic groups, e.g. cycloalkyl groups. Examples of such cycloalkyl groups include cyclopropyl, cyclobutyl, cyclopentyl, cyclohexyl and cycloheptyl; more typically cyclopropyl, cyclobutyl, cyclopentyl and cyclohexyl, particularly cyclohexyl.

Further examples of non-aromatic cyclic groups include bridged ring systems such as bicycloalkanes and azabicycloalkanes although such bridged ring systems are generally less preferred. By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged ring systems include bicyclo[2.2.1]heptane, aza-bicyclo[2.2.1]heptane, bicyclo[2.2.2]octane, aza-bicyclo[2.2.2]octane, bicyclo[3.2.1]octane and aza-bicyclo[3.2.1]octane. A particular example of a bridged ring system is the 1-aza-bicyclo[2.2.2]octan-3-yl group.

Where reference is made herein to carbocyclic and heterocyclic groups, the carbocyclic or heterocyclic ring can, unless the context indicates otherwise, be unsubstituted or substituted by one or more substituent groups R¹⁰ selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;

R^{c} is selected from hydrogen and C₁₋₄ hydrocarbyl; and

X¹ is O, S or NR^{c} and X² is ═O, ═S or ═NR^{c}.

Where the substituent group R¹⁰ comprises or includes a carbocyclic or heterocyclic group, the said carbocyclic or heterocyclic group may be unsubstituted or may itself be substituted with one or more further substituent groups R¹⁰. In one sub-group of compounds of the formula (I), such further substituent groups R¹⁰ may include carbocyclic or heterocyclic groups, which are typically not themselves further substituted. In another sub-group of compounds of the formula (I), the said further substituents do not include carbocyclic or heterocyclic groups but are otherwise selected from the groups listed above in the definition of R¹⁰.

The substituents R¹⁰ may be selected such that they contain no more than 20 non-hydrogen atoms, for example, no more than 15 non-hydrogen atoms, e.g. no more than 12, or 11, or 10, or 9, or 8, or 7, or 6, or 5 non-hydrogen atoms.

Where the carbocyclic and heterocyclic groups have a pair of substituents on adjacent ring atoms, the two substituents may be linked so as to form a cyclic group. Thus, two adjacent groups R¹⁰, together with the carbon atoms or heteroatoms to which they are attached may form a 5-membered heteroaryl ring or a 5- or 6-membered non-aromatic carbocyclic or heterocyclic ring, wherein the said heteroaryl and heterocyclic groups contain up to 3 heteroatom ring members selected from N, O and S. For example, an adjacent pair of substituents on adjacent carbon atoms of a ring may be linked via one or more heteroatoms and optionally substituted alkylene groups to form a fused oxa-, dioxa-, aza-, diaza- or oxa-aza-cycloalkyl group.

Examples of such linked substituent groups include:

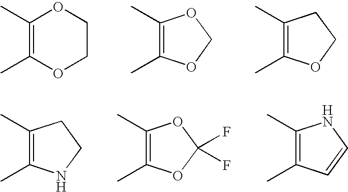

Examples of halogen substituents include fluorine, chlorine, bromine and iodine. Fluorine and chlorine are particularly preferred.

In the definition of the compounds of the formula (I) above and as used hereinafter, the term “hydrocarbyl” is a generic term encompassing aliphatic, alicyclic and aromatic groups having an all-carbon backbone and consisting of carbon and hydrogen atoms, except where otherwise stated.

In certain cases, as defined herein, one or more of the carbon atoms making up the carbon backbone may be replaced by a specified atom or group of atoms.

Examples of hydrocarbyl groups include alkyl, cycloalkyl, cycloalkenyl, carbocyclic aryl, alkenyl, alkynyl, cycloalkylalkyl, cycloalkenylalkyl, and carbocyclic aralkyl, aralkenyl and aralkynyl groups. Such groups can be unsubstituted or, where stated, substituted by one or more substituents as defined herein. The examples and preferences expressed below apply to each of the hydrocarbyl substituent groups or hydrocarbyl-containing substituent groups referred to in the various definitions of substituents for compounds of the formula (I), (Ia), (Ib), (Ic), (Id), (Ie), (II), (IIa), (IIb), (III), (IIIa), (IIIb), (IV), (V), (Va), (Vb), (VI), (VIa), (VII) and (VIII) unless the context indicates otherwise.

Preferred non-aromatic hydrocarbyl groups are saturated groups such as alkyl and cycloalkyl groups.

Generally by way of example, the hydrocarbyl groups can have up to eight carbon atoms, unless the context requires otherwise. Within the sub-set of hydrocarbyl groups having 1 to 8 carbon atoms, particular examples are C₁₋₆ hydrocarbyl groups, such as C₁₋₄ hydrocarbyl groups (e.g. C₁₋₃ hydrocarbyl groups or C₁₋₂ hydrocarbyl groups), specific examples being any individual value or combination of values selected from C₁, C₂, C₃, C₄, C₅, C₆, C₇ and C₈ hydrocarbyl groups.

The term “alkyl” covers both straight chain and branched chain alkyl groups. Examples of alkyl groups include methyl, ethyl, propyl, isopropyl, n-butyl, isobutyl, tert-butyl, n-pentyl, 2-pentyl, 3-pentyl, 2-methyl butyl, 3-methyl butyl, and n-hexyl and its isomers. Within the sub-set of alkyl groups having 1 to 8 carbon atoms, particular examples are C₁₋₆ alkyl groups, such as C₁₋₄ alkyl groups (e.g. C₁₋₃ alkyl groups or C₁₋₂ alkyl groups).

Examples of cycloalkyl groups are those derived from cyclopropane, cyclobutane, cyclopentane, cyclohexane and cycloheptane. Within the sub-set of cycloalkyl groups the cycloalkyl group will have from 3 to 8 carbon atoms, particular examples being C₃₋₆ cycloalkyl groups.

Examples of alkenyl groups include, but are not limited to, ethenyl (vinyl), 1-propenyl, 2-propenyl (allyl), isopropenyl, butenyl, buta-1,4-dienyl, pentenyl, and hexenyl. Within the sub-set of alkenyl groups the alkenyl group will have 2 to 8 carbon atoms, particular examples being C₂₋₆ alkenyl groups, such as C₂₋₄ alkenyl groups.

Examples of cycloalkenyl groups include, but are not limited to, cyclopropenyl, cyclobutenyl, cyclopentenyl, cyclopentadienyl and cyclohexenyl. Within the sub-set of cycloalkenyl groups the cycloalkenyl groups have from 3 to 8 carbon atoms, and particular examples are C₃₋₆ cycloalkenyl groups.

Examples of alkynyl groups include, but are not limited to, ethynyl and 2-propynyl (propargyl) groups. Within the sub-set of alkynyl groups having 2 to 8 carbon atoms, particular examples are C₂₋₆ alkynyl groups, such as C₂₋₄ alkynyl groups.

Examples of carbocyclic aryl groups include substituted and unsubstituted phenyl groups.

Examples of cycloalkylalkyl, cycloalkenylalkyl, carbocyclic aralkyl, aralkenyl and aralkynyl groups include phenethyl, benzyl, styryl, phenylethynyl, cyclohexylmethyl, cyclopentylmethyl, cyclobutylmethyl, cyclopropylmethyl and cyclopentenylmethyl groups.

When present, and where stated, a hydrocarbyl group can be optionally substituted by one or more substituents selected from hydroxy, oxo, alkoxy, carboxy, halogen, cyano, nitro, amino, mono- or di-C₁₋₄ hydrocarbylamino, and monocyclic or bicyclic carbocyclic and heterocyclic groups having from 3 to 12 (typically 3 to 10 and more usually 5 to 10) ring members. Preferred substituents include halogen such as fluorine. Thus, for example, the substituted hydrocarbyl group can be a partially fluorinated or perfluorinated group such as difluoromethyl or trifluoromethyl. In one embodiment preferred substituents include monocyclic carbocyclic and heterocyclic groups having 3-7 ring members, more usually 3, 4, 5 or 6 ring members.

Where stated, one or more carbon atoms of a hydrocarbyl group may optionally be replaced by O, S, SO, SO₂, NR^{c}, X¹C(X²), C(X²)X or X¹C(X²)X¹ (or a sub-group thereof) wherein X¹ and X² are as hereinbefore defined, provided that at least one carbon atom of the hydrocarbyl group remains. For example, 1, 2, 3 or 4 carbon atoms of the hydrocarbyl group may be replaced by one of the atoms or groups listed, and the replacing atoms or groups may be the same or different. In general, the number of linear or backbone carbon atoms replaced will correspond to the number of linear or backbone atoms in the group replacing them. Examples of groups in which one or more carbon atom of the hydrocarbyl group have been replaced by a replacement atom or group as defined above include ethers and thioethers (C replaced by O or S), amides, esters, thioamides and thioesters (C—C replaced by X¹C(X²) or C(X²)X¹), sulphones and sulphoxides (C replaced by SO or SO₂), amines (C replaced by NR^{c}). Further examples include ureas, carbonates and carbamates (C—C—C replaced by X¹C(X²)X¹).

Where an amino group has two hydrocarbyl substituents, they may, together with the nitrogen atom to which they are attached, and optionally with another heteroatom such as nitrogen, sulphur, or oxygen, link to form a ring structure of 4 to 7 ring members, more usually 5 to 6 ring members.

The term “aza-cycloalkyl” as used herein refers to a cycloalkyl group in which one of the carbon ring members has been replaced by a nitrogen atom. Thus examples of aza-cycloalkyl groups include piperidine and pyrrolidine. The term “oxa-cycloalkyl” as used herein refers to a cycloalkyl group in which one of the carbon ring members has been replaced by an oxygen atom. Thus examples of oxa-cycloalkyl groups include tetrahydrofuran and tetrahydropyran. In an analogous manner, the terms “diaza-cycloalkyl”, “dioxa-cycloalkyl” and “aza-oxa-cycloalkyl” refer respectively to cycloalkyl groups in which two carbon ring members have been replaced by two nitrogen atoms, or by two oxygen atoms, or by one nitrogen atom and one oxygen atom.

The definition “R^{a}—R^{b}” as used herein, either with regard to substituents present on a carbocyclic or heterocyclic moiety, or with regard to other substituents present at other locations on the compounds of the formula (I), includes inter alia compounds wherein R^{a} is selected from a bond, O, CO, OC(O), SC(O), NR^{c}C(O), OC(S), SC(S), NR^{c}C(S), OC(NR^{c}), SC(NR^{c}), NR^{c}C(NR^{c}), C(O)O, C(O)S, C(O)NR^{c}, C(S)O, C(S)S, C(S)NR^{c}, C(NR^{c})O, C(NR^{c})S, C(NC)NR^{c}, OC(O)O, SC(O)O, NR^{c}C(O)O, OC(S)O, SC(S)O, NR^{c}C(S)O, OC(NR^{c})O, SC(NR^{c})O, NR^{c}C(NR^{c})O, OC(O)S, SC(O)S, NR^{c}C(O)S, OC(S)S, SC(S)S, NR^{c}C(S)S, OC(NR^{c})S, SC(NR^{c})S, NR^{c}C(R^{c}C)S, OC(O)NR^{c}, SC(O)NR^{c}, NR^{c}C(O)NR^{c}, OC(S)NR^{c}, SC(S)NR^{c}, NR^{c}C(S)NR^{c}, OC(NR^{c})NR^{c}, SC(NR^{c})NR^{c}, NR^{c}C(NR^{c}NR^{c}, S, SO, SO₂ NR^{c}, SO₂NR^{c} and NR^{c}SO₂ wherein R^{c} is as hereinbefore defined.

The moiety R^{b} can be hydrogen or it can be a group selected from carbocyclic and heterocyclic groups having from 3 to 12 ring members (typically 3 to 10 and more usually from 5 to 10), and a C₁₋₈ hydrocarbyl group optionally substituted as hereinbefore defined. Examples of hydrocarbyl, carbocyclic and heterocyclic groups are as set out above.

When R^{a} is O and R^{b} is a C₁₋₈ hydrocarbyl group, R^{a} and R^{b} together form a hydrocarbyloxy group. Preferred hydrocarbyloxy groups include saturated hydrocarbyloxy such as alkoxy (e.g. C₁₋₆ alkoxy, more usually C₁₋₄ alkoxy such as ethoxy and methoxy, particularly methoxy), cycloalkoxy (e.g. C₃₋₆ cycloalkoxy such as cyclopropyloxy, cyclobutyloxy, cyclopentyloxy and cyclohexyloxy) and cycloalkyalkoxy (e.g. C₃₋₆ cycloalkyl-C₁₋₂ alkoxy such as cyclopropylmethoxy).

The hydrocarbyloxy groups can be substituted by various substituents as defined herein. For example, the alkoxy groups can be substituted by halogen (e.g. as in difluoromethoxy and trifluoromethoxy), hydroxy (e.g. as in hydroxyethoxy), C₁₋₂ alkoxy (e.g. as in methoxyethoxy), hydroxy-C₁₋₂ alkyl (as in hydroxyethoxyethoxy) or a cyclic group (e.g. a cycloalkyl group or non-aromatic heterocyclic group as hereinbefore defined). Examples of alkoxy groups bearing a non-aromatic heterocyclic group as a substituent are those in which the heterocyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkoxy group is a C₁₋₄ alkoxy group, more typically a C₁₋₃ alkoxy group such as methoxy, ethoxy or n-propoxy. Particular examples are alkoxy groups substituted by a monocyclic group such as pyrrolidine, piperidine, morpholine and piperazine and N-substituted derivatives thereof such as N-benzyl, N—C₁₋₄ acyl and N—C₁₋₄ alkoxycarbonyl, for example pyrrolidinoethoxy, piperidinoethoxy and piperazinoethoxy.

When R^{a} is a bond and R^{b} is a C₁₋₈ hydrocarbyl group, examples of hydrocarbyl groups R^{a}—R^{b} are as hereinbefore defined. The hydrocarbyl groups may be saturated groups such as cycloalkyl and alkyl and particular examples of such groups include methyl, ethyl and cyclopropyl. The hydrocarbyl (e.g. alkyl) groups can be substituted by various groups and atoms as defined herein. Examples of substituted alkyl groups include alkyl groups substituted by one or more halogen atoms such as fluorine and chlorine (particular examples including bromoethyl, chloroethyl and trifluoromethyl), or hydroxy (e.g. hydroxymethyl and hydroxyethyl), C₁₋₈ acyloxy (e.g. acetoxymethyl and benzyloxymethyl), amino and mono- and dialkylamino (e.g. aminoethyl, methylaminoethyl, dimethylaminomethyl, dimethylaminoethyl and tert-butylaminoethyl), alkoxy (e.g. C₁₋₂ alkoxy such as methoxy—as in methoxyethyl), and cyclic groups such as cycloalkyl groups, aryl groups, heteroaryl groups and non-aromatic heterocyclic groups as hereinbefore defined).

Particular examples of alkyl groups substituted by a cyclic group are those wherein the cyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkyl group is a C₁₋₄ alkyl group, more typically a C₁₋₃ alkyl group such as methyl, ethyl or n-propyl. Specific examples of alkyl groups substituted by a cyclic group include pyrrolidinomethyl, pyrrolidinopropyl, morpholinomethyl, morpholinoethyl, morpholinopropyl, piperidinylmethyl, piperazinomethyl and N-substituted forms thereof as defined herein.

Particular examples of alkyl groups substituted by aryl groups and heteroaryl groups include benzyl and pyridylmethyl groups.

When R^{a} is SO₂NR^{c}, R^{b} can be, for example, hydrogen or an optionally substituted C₁₋₈ hydrocarbyl group, or a carbocyclic or heterocyclic group. Examples of R^{a}—R^{b} where R^{a} is SO₂NR^{c} include aminosulphonyl, C₁₋₄ alkylaminosulphonyl and di-C₁₋₄ alkylaminosulphonyl groups, and sulphonamides formed from a cyclic amino group such as piperidine, morpholine, pyrrolidine, or an optionally N-substituted piperazine such as N-methyl piperazine.

Examples of groups R^{a}—R^{b} where R^{a} is SO₂ include alkylsulphonyl, heteroarylsulphonyl and arylsulphonyl groups, particularly monocyclic aryl and heteroaryl sulphonyl groups. Particular examples include methylsulphonyl, phenylsulphonyl and toluenesulphonyl.

When R^{a} is NR^{c}, R^{b} can be, for example, hydrogen or an optionally substituted C₁₋₈ hydrocarbyl group, or a carbocyclic or heterocyclic group. Examples of R^{a}—R^{b} where R^{a} is NR^{c} include amino, C₁₋₄ alkylamino (e.g. methylamino, ethylamino, propylamino, isopropylamino, tert-butylamino), di-C₁₋₄ alkylamino (e.g. dimethylamino and diethylamino) and cycloalkylamino (e.g. cyclopropylamino, cyclopentylamino and cyclohexylamino).

### Specific Embodiments of and Preferences for Y, A, R^{g}, R¹ to R⁴ and R¹⁰

### A

In formula (I), A is a bond, C═O, NR^{g}(C═O) or O(C═O). It will be appreciated that the moiety R¹-A-NR⁴ linked to the 4-position of the isothiazole or thiazole ring can therefore take the form of an amine R¹—NR⁴, an amide R¹—C(═O)NR⁴, a urea R¹—NR^{g}C(═O)NR⁴ or a carbamate R¹—OC(═O)NR⁴.

In one preferred group of compounds of the invention, A is C═O and hence the group R¹-A-NR⁴ takes the form of an amide R¹—C(═O)NR⁴.

In another group of compounds of the invention, A is a bond and hence the group R¹-A-NR⁴ takes the form of an amine R¹—NR⁴.

In a further group of compounds of the invention, A is NR^{g}(C═O) and hence the group R¹-A-NR⁴ takes the form of a urea. Within this group of compounds, R^{g} is typically hydrogen or methyl. In one embodiment, R^{g} is hydrogen in another embodiment R^{g} is methyl.

### R⁴

R⁴ is hydrogen or a C₁₋₄ hydrocarbyl group optionally substituted by halogen (e.g. fluorine), hydroxyl or C₁₋₄ alkoxy (e.g. methoxy).

The number of optional substitutents on the hydrocarbyl group typically will vary according to the nature of the substituent. For example, where the substituent is halogen, there may be from one to three halogen atoms present, preferably two or three. Where the substituent is hydroxyl or an alkoxy group, typically there will be only a single such substituent present

R⁴ is preferably hydrogen or C₁₋₃ alkyl, more preferably hydrogen or methyl and most preferably is hydrogen.

### R^{g}

R^{g} is hydrogen or a C₁₋₄ hydrocarbyl group optionally substituted by hydroxyl or C₁₋₄ alkoxy (e.g. methoxy).

When R^{g} is C₁₋₄ hydrocarbyl substituted by hydroxyl or C₁₋₄ alkoxy, typically there is only one such substituent present.

Preferably R^{g} is hydrogen or C₁₋₃ alkyl, more preferably hydrogen or methyl and most preferably R^{g} is hydrogen.

### R²

R² is hydrogen, halogen, C₁₋₄ alkoxy, or a C₁₋₄ hydrocarbyl group optionally substituted by halogen, hydroxyl or C₁₋₄ alkoxy.

When R² is halogen, preferably it is selected from chlorine and fluorine and more preferably it is fluorine.

When R¹ is C₁₋₄ alkoxy, it can be, for example, C₁₋₁₃ alkoxy, more preferably C₁₋₂ alkoxy and most preferably methoxy.

When R² is an optionally substituted C₁₋₄ hydrocarbyl group, the hydrocarbyl group is preferably a C₁₋₃ hydrocarbyl group, more preferably a C₁₋₂ hydrocarbyl group, for example an optionally substituted methyl group. The optional substituents for the optionally substituted hydrocarbyl group are preferably selected from fluorine, hydroxyl and methoxy.

The number of optional substituents on the hydrocarbyl group typically will vary according to the nature of the substituent. For example, where the substituent is halogen, there may be from one to three halogen atoms present, preferably two or three. Where the substituent is hydroxyl or methoxy, typically there will be only a single such substituent present.

The hydrocarbyl groups constituting R² are preferably saturated hydrocarbyl groups. Examples of saturated hydrocarbyl groups include methyl, ethyl, n-propyl, i-propyl and cyclopropyl.

In one embodiment, R² is hydrogen, halogen, C₁₋₄ alkoxy, or a C₁₋₄ hydrocarbyl group optionally substituted by halogen, hydroxyl or C₁₋₄ alkoxy.

In another embodiment, R² is hydrogen, fluorine, chlorine, methoxy, or a C₁₋₃ hydrocarbyl group optionally substituted by fluorine, hydroxyl or methoxy.

In a preferred embodiment, R² is hydrogen or methyl, most preferably hydrogen.

### R¹

In formulae (I), (Ib) and (Id), R¹ is hydrogen, a carbocyclic or heterocyclic group having from 3 to 12 ring members, or a C₁₋₈ hydrocarbyl group optionally substituted by one or more substituents selected from halogen (e.g. fluorine), hydroxy, C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂. Examples of carbocyclic or heterocyclic groups and hydrocarbyl groups and general preferences for such groups are as set out above in the General Preferences and Definitions section, and as set out below.

A C₁₋₈ hydrocarbyl group R¹ can be optionally substituted by one or more substituents selected from halogen (e.g. fluorine), hydroxy, C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂. Particular substituents for the hydrocarbyl group include hydroxy, chlorine, fluorine (e.g. as in trifluoromethyl), methoxy, ethoxy, amino, methylamino and dimethylamino, preferred substituents being hydroxy and fluorine.

In formulae (Ia), (Ic) and (Ie), R¹ is (i) a carbocyclic or heterocyclic group having from 3 to 12 ring members; or (ii) a C₁₋₄ hydrocarbyl group substituted by a carbocyclic or heterocyclic group having from 3 to 12 ring members, the hydrocarbyl group being optionally further substituted by one or more substituents selected from halogen (e.g. fluorine), hydroxy, C₁₋₄ hydrocarbyloxy, amino, mono- or di-C₁₋₄ hydrocarbylamino, and wherein 1 or 2 of the carbon atoms of the hydrocarbyl group may optionally be replaced by an atom or group selected from O, S, NH, SO and SO₂.

In one embodiment, R¹ is (i) a carbocyclic or heterocyclic group having from 3 to 12 ring members.

In another embodiment, R¹ is (ii) a C₁₋₄ hydrocarbyl group substituted by a carbocyclic or heterocyclic group having from 3 to 12 ring members, the hydrocarbyl group being optionally further substituted by one or more substituents selected from halogen (e.g. fluorine), hydroxy, C₁₋₄ hydrocarbyloxy, amino, mono- or di-C₁₋₄ hydrocarbylamino, and wherein 1 or 2 of the carbon atoms of the hydrocarbyl group may optionally be replaced by an atom or group selected from O, S, NH, SO and SO₂.

Particular examples of C₁₋₄ hydrocarbyl group substituted by a carbocyclic or heterocyclic group are C₁₋₂ alkyl groups substituted by an optionally substituted phenyl or pyridyl group, for example an optionally substituted benzyl, phenylethyl (e.g. 1-phenylethyl) or pyridyl-methyl group. One preferred group is an optionally substituted benzyl group.

The following preferences, definitions and examples for carbocyclic and heterocyclic groups apply individually to each of formulae (I), (Ia), (Ib), (Ic), (Id), (Ie), (II), (IIa), (IIb), (III), (IIa), (IIIb), (IV), (V), (Va), (Vb), (VI), (VIa), (VII) and (VIII) and sub-groups thereof, such as sub-groups (i) and (ii) of the group R¹ in formulae (Ia), (Ic) and (Ie).

Examples of carbocyclic or heterocyclic groups and hydrocarbyl groups and general preferences for such groups are as set out above in the General Preferences and Definitions section, and as set out below.

In one embodiment, the carbocyclic or heterocyclic group is an aryl or heteroaryl group.

The aryl and heteroaryl groups are typically monocyclic or bicyclic and preferably are monocyclic.

When the carbocyclic or heterocyclic group is a heteroaryl group, particular heteroaryl groups include monocyclic heteroaryl groups containing up to three heteroatom ring members selected from O, S and N, and bicyclic heteroaryl groups containing up to 2 heteroatom ring members selected from O, S and N and wherein both rings are aromatic.

Examples of such groups include furanyl (e.g. 2-furanyl or 3-furanyl), indolyl (e.g. 3-indolyl, 6-indolyl), 2,3-dihydro-benzo[1,4]dioxinyl (e.g. 2,3-dihydro-benzo[1,4]dioxin-5-yl), pyrazolyl (e.g. pyrazole-5-yl), pyrazolo[1,5-a]pyridinyl (e.g. pyrazolo[1,5-a]pyridine-3-yl), oxazolyl (e.g.), isoxazolyl (e.g. isoxazol-4-yl), pyridyl (e.g. 2-pyridyl, 3-pyridyl, 4-pyridyl), quinolinyl (e.g. 2-quinolinyl), pyrrolyl (e.g. 3-pyrrolyl), imidazolyl and thienyl (e.g. 2-thienyl, 3-thienyl).

One sub-group of heteroaryl groups consists of furanyl (e.g. 2-furanyl or 3-furanyl), indolyl, oxazolyl, isoxazolyl, pyridyl, quinolinyl, pyrrolyl, imidazolyl and thienyl.

A particular sub-set of heteroaryl groups includes 2-furanyl, 3-furanyl, pyrrolyl, imidazolyl and thienyl.

Preferred aryl groups are phenyl groups.

Further preferred groups are benzyl groups.

The carbocyclic or heterocyclic group (whether directly attached to ANR⁴ or linked via a hydrocarbyl group) can be an unsubstituted or substituted carbocylic or heterocyclic group in which one or more substituents can be selected from the group R¹⁰ as hereinbefore defined. In one embodiment, the substituents on the carbocyclic or heterocyclic group may be selected from the group R^{10a} consisting of halogen, hydroxy, trifluoromethyl, cyano, nitro, carboxy, a group R^{a}—R^{b} wherein R^{a} is a bond, O, CO, X³C(X⁴), C(X⁴)X³, X³C(X⁴)X³, S, SO, or SO₂, and R^{b} is selected from hydrogen and a C₁₋₈ hydrocarbyl group optionally substituted by one or more substituents selected from hydroxy, oxo, halogen, cyano, nitro, carboxy and monocyclic non-aromatic carbocyclic or heterocyclic groups having from 3 to 6 ring members; wherein one or more carbon atoms of the C₁₋₈ hydrocarbyl group may optionally be replaced by O, S, SO, SO₂, X³C(X⁴), C(X⁴)X³ or X³C(X⁴)X³; X³ is O or S; and X⁴ is ═O or ═S.

Where the carbocyclic and heterocyclic groups have a pair of substituents on adjacent ring atoms, the two substituents may be linked so as to form a cyclic group. Thus, two adjacent groups R¹⁰, together with the carbon atoms or heteroatoms to which they are attached may form a 5-membered heteroaryl ring or a 5- or 6-membered non-aromatic carbocyclic or heterocyclic ring, wherein the said heteroaryl and heterocyclic groups contain up to 3 heteroatom ring members selected from N, O and S. In particular the two adjacent groups R¹⁰, together with the carbon atoms or heteroatoms to which they are attached, may form a 6-membered non-aromatic heterocyclic ring, containing up to 3, in particular 2, heteroatom ring members selected from N, O and S. More particularly the two adjacent groups R¹⁰ may form a 6-membered non-aromatic heterocyclic ring, containing 2 heteroatom ring members selected from N, or O, such as dioxan e.g. [1,4 dioxan]. In one embodiment the carbocyclic or heterocyclic group is a carbocyclic group e.g. phenyl having a pair of substituents on adjacent ring atoms linked so as to form a cyclic group e.g. to form 2,3-dihydro-benzo[1,4]dioxine.

In another embodiment, two adjacent groups R¹⁰ do not link to form a cyclic group.

More particularly, the substituents on the carbocyclic or heterocyclic group may be selected from halogen, hydroxy, trifluoromethyl, a group R^{a}—R^{b} wherein R^{a} is a bond or O, and R^{b} is selected from hydrogen and a C₁₋₄ hydrocarbyl group optionally substituted by one or more substituents selected from hydroxyl, halogen (preferably fluorine) and 5 and 6 membered saturated carbocyclic and heterocyclic groups (for example groups containing up to two heteroatoms selected from O, S and N, such as unsubstituted piperidine, pyrrolidino, morpholino, piperazino and N-methyl piperazino).

The carbocyclic or heterocyclic group may be substituted by more than one substituent. Thus, for example, there may be 1 or 2 or 3 or 4 substituents. In one embodiment, where the carbocyclic or heterocyclic group is a six membered ring (e.g. a carbocyclic ring such as a phenyl ring), there may be one, two or three substituents and these may be located at the 2-, 3-, 4- or 6-positions around the ring. By way of example, a phenyl group may be 2-monosubstituted, 3-monosubstituted, 2,6-disubstituted, 2,3-disubstituted, 2,4-disubstituted 2,5-disubstituted, 2,3,6-trisubstituted or 2,4,6-trisubstituted. More particularly, a phenyl group may be monosubstituted at the 2-position or disubstituted at positions 2- and 6- with substituents selected from fluorine, chlorine and R^{a}—R^{b}, where R^{a} is O and R^{b} is C₁₋₄ alkyl (e.g. methyl or ethyl). In one embodiment, fluorine is a preferred substituent. In another embodiment, preferred substituents are selected from fluorine, chlorine and methoxy.

In the context of R¹, particular examples of non-aromatic groups include unsubstituted or substituted (by one or more groups R¹⁰) monocyclic cycloalkyl groups. Examples of such cycloalkyl groups include cyclopropyl, cyclobutyl, cyclopentyl, cyclohexyl and cycloheptyl; more typically cyclopropyl, cyclobutyl, cyclopentyl and cyclohexyl, particularly cyclohexyl.

Further examples of non-aromatic groups include unsubstituted or substituted (by one or more groups R¹⁰) heterocyclic groups having from 3 to 12 ring members, typically 4 to 12 ring members, and more usually from 5 to 10 ring members. Such groups can be monocyclic or bicyclic, for example, and typically have from 1 to 5 heteroatom ring members (more usually 1, 2, 3 or 4 heteroatom ring members) typically selected from nitrogen, oxygen and sulphur.

When sulphur is present, it may, where the nature of the adjacent atoms and groups permits, exist as —S—, —S(O)— or —S(O)₂—.

The heterocylic groups can contain, for example, cyclic ether moieties (e.g. as in tetrahydrofuran and dioxane), cyclic thioether moieties (e.g. as in tetrahydrothiophene and dithiane), cyclic amine moieties (e.g. as in pyrrolidine), cyclic amides (e.g. as in pyrrolidone), cyclic esters (e.g. as in butyrolactone), cyclic thioamides and thioesters, cyclic sulphones (e.g. as in sulpholane and sulpholene), cyclic sulphoxides, cyclic sulphonamides and combinations thereof (e.g. morpholine and thiomorpholine and its S-oxide and S,S-dioxide).

In the context of R¹, examples of monocyclic non-aromatic heterocyclic groups include 5-, 6- and 7-membered monocyclic heterocyclic groups such as morpholine, piperidine (e.g. 1-piperidinyl, 2-piperidinyl 3-piperidinyl and 4-piperidinyl), pyrrolidine (e.g. 1-pyrrolidinyl, 2-pyrrolidinyl and 3-pyrrolidinyl), pyrrolidone, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), tetrahydrothiopyran (e.g. 4-tetrahydrothiopyran), imidazoline, imidazolidinone, oxazoline, thiazoline, 2-pyrazoline, pyrazolidine, piperazine, N-alkyl piperazines such as N-methyl piperazine, thiomorpholine and its S-oxide and S,S-dioxide (particularly thiomorpholine), and N-alkyl piperidines such as N-methyl piperidine.

In general, preferred non-aromatic heterocyclic groups include pyrrolidine, piperidine, tetrahydropyran, tetrahydrothiopyran, morpholine, thiomorpholine, thiomorpholine S,S-dioxide, piperazine, N-alkyl piperazines, and N-alkyl piperidines.

One sub-group of non-aromatic heterocyclic groups includes pyrrolidine, piperidine, morpholine, thiomorpholine, thiomorpholine S,S-dioxide, piperazine, N-alkyl piperazines, and N-alkyl piperidines.

When R¹ is a C₁₋₄ hydrocarbyl group substituted by a carbocyclic or heterocyclic group, the substituted hydrocarbyl group is typically a saturated C₁₋₄ hydrocarbyl group and is preferably up to three carbon atoms in length for example one or two carbon atoms in length. Particular examples are the alkylene groups CH₂ and CH₂CH₂. A further particular example is the group CH—CH₃. Where the substituted hydrocarbyl group is a C₂₋₄ hydrocarbyl group, one of the carbon atoms and its associated hydrogen atoms may be replaced by a sulphonyl group, for example as in the moiety SO₂CH₂.

Alternatively, the carbocyclic group can be an aryl or heteroaryl group as defined herein, and in particular an optionally substituted phenyl group in which the optional substituents are selected from the group R¹⁰ as defined herein.

The hydrocarbyl group may be a straight chain or branched alkyl group. Examples of straight chain alkyl groups include groups derived from methyl, ethyl, propyl and butyl groups.

Examples of branched chain alkyl groups include those derived from isopropyl, isobutyl, tert-butyl and 2,2-dimethylpropyl groups.

The C₁₋₄ hydrocarbyl group can be additionally substituted by one or more substituents selected from halogen (e.g. fluorine), hydroxy, C₁₋₄ hydrocarbyloxy, amino, and mono- or di-C₁₋₄ hydrocarbylamino, and wherein 1 or 2 of the carbon atoms of the hydrocarbyl group may optionally be replaced by an atom or group selected from O, S, NH, SO, SO₂. Particular substituents for the hydrocarbyl group include hydroxy, chlorine, fluorine (e.g. as in trifluoromethyl), methoxy, ethoxy, amino, methylamino and dimethylamino, preferred substituents being hydroxy and fluorine. In a further embodiment, the C₁₋₄ hydrocarbyl group can be additionally substituted by a cyano group.

In one embodiment, R¹ is a group R^{1a}—(V)ₙ— where:

n is 0 or 1;

V is selected from CH₂, CH(CH₃), CH₂CH₂ and SO₂CH₂ (for example from CH₂, CH₂CH₂ and SO₂CH₂); and

R^{1a} is a carbocyclic or heterocyclic group selected from phenyl;

five membered heteroaryl rings having up to 4 heteroatom ring members selected from N, O and S;

six membered heteroaryl rings containing one or two nitrogen ring members; five or six membered saturated non-aromatic heterocyclic rings containing one or two heteroatom ring members selected from N, O, S and SO₂;

C₃₋₆ cycloalkyl groups; indole; and quinoline;

wherein each of the carbocyclic and heterocyclic groups R^{1a} can be optionally substituted by one or more substituents selected from five or six membered saturated non-aromatic carbocyclic and heterocyclic groups containing up to two heteroatom ring members selected from N, O, S and SO₂; hydroxy; amino; oxo; mono-C₁₋₄ alkylamino; di-C₁₋₄ alkylamino; fluorine; chlorine; nitro; C₁₋₄ alkyl-(O)_{q}— wherein q is 0 or 1 and the C₁₋₄ alkyl moiety is optionally substituted by fluorine, hydroxy, C₁₋₂ alkoxy or a five or six membered saturated non-aromatic carbocyclic or heterocyclic group containing up to two heteroatom ring members selected from N, O, S and SO₂; phenyl and C₁₋₂-alkylene dioxy.

When A is C═O, particular groups R¹—CO are the groups set out in Table 1 below.

In Table 1, the point of attachment of the group to the nitrogen atom of the isothiazole-4-amino group is represented by the terminal single bond extending from the carbonyl group. Thus, by way of illustration, group D in the table is the phenylacetyl group and group I in the table is the 3-(4-chlorophenyl)propionyl group.

TABLE 1
Examples of the group R¹-CO 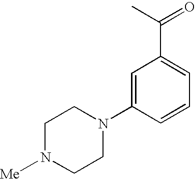
A 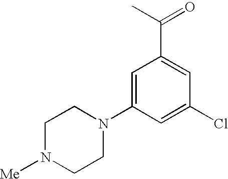
B 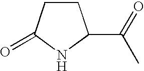
C 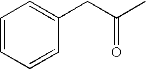
D 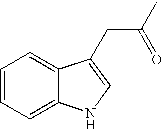
E 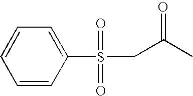
F 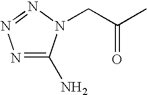
G 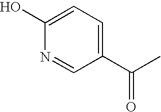
H 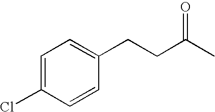
I 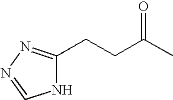
J 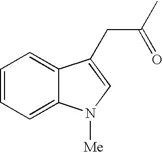
K 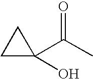
L 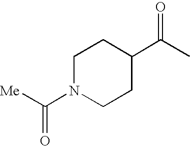
M 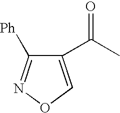
N 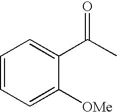
O 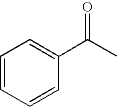
P 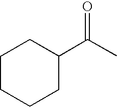
Q 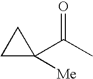
R 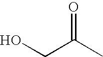
S 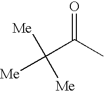
T 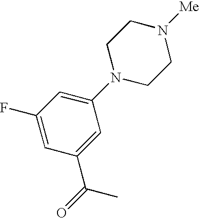
U 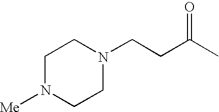
V 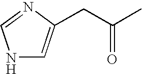
W 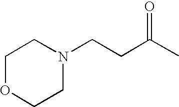
X 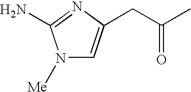
Y 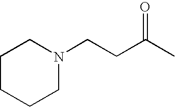
Z 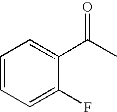
AA 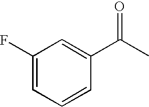
AB 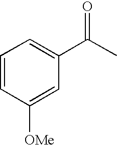
AC 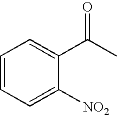
AD 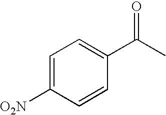
AE 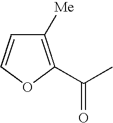
AF 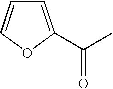
AG 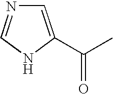
AH 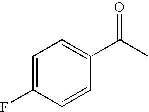
AI 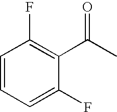
AJ 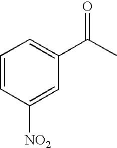
AK 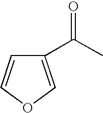
AL 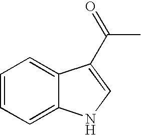
AM 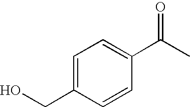
AN 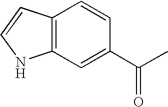
AO 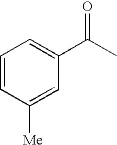
AP 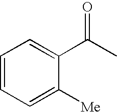
AQ 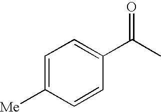
AR 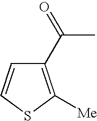
AS 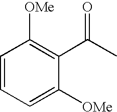
AT 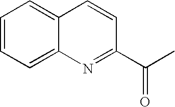
AU 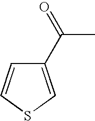
AV 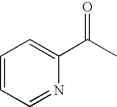
AW 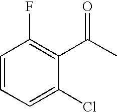
AX 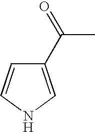
AY 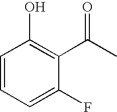
AZ 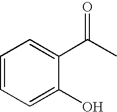
BA 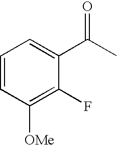
BB 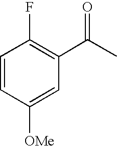
BC 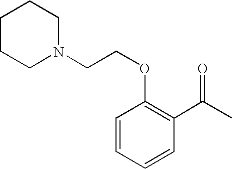
BD 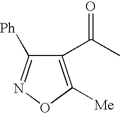
BE 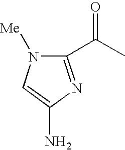
BF 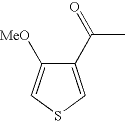
BG 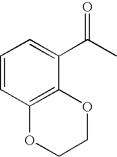
BH 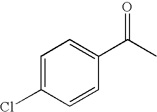
BI 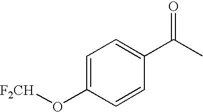
BJ 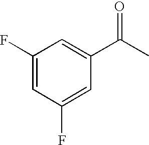
BK 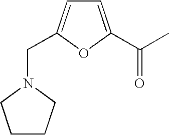
BL 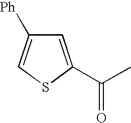
BM 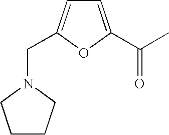
BN 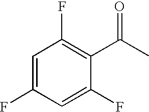
BO 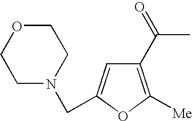
BP 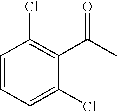
BQ 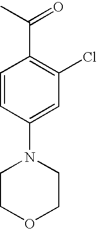
BR 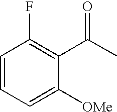
BS 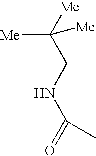
BT 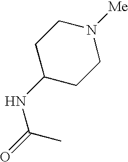
BU 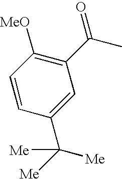
BV 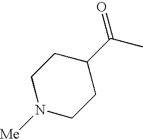
BW 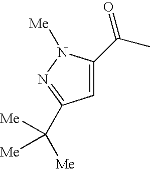
BX 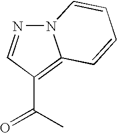
BY 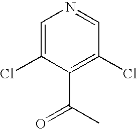
BZ 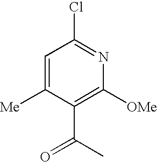
BAA 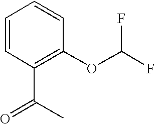
BAB 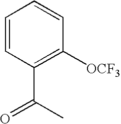
BAC 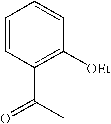
BAD 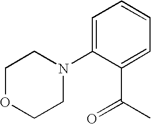
BAE 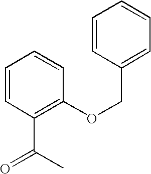
BAF 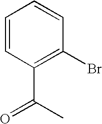
BAG 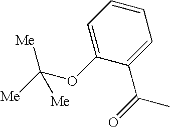
BAH 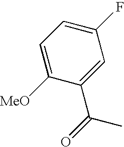
BAI 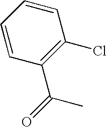
BAJ 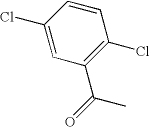
BAK 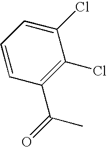
BAL 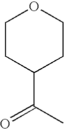
BAM 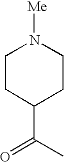
BAN 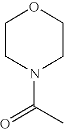
BAO 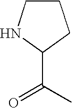
BAP 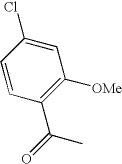
BAQ 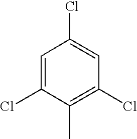
BAR 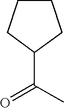
BAS

One sub-set of groups consists of groups A to BAL.

Another sub-set of groups consists of groups A to BAQ.

One particularly preferred sub-set of groups R¹—CO— consists of AJ, BQ and BS, and in particular AJ and BQ, more particularly BQ.

A further sub-set of groups consists of groups P, AJ, BQ, BAR, BAS and BAD.

When A is C═O, and R¹ is a phenyl ring bearing a substituent at the 4-position, the substituent at the 4-position is preferably other than a phenyl group having a group SO₂NH₂ or SO₂Me at the ortho-position.

When A is a bond, R¹ can be any of the groups R¹ set out in Table 1 for compounds where A is C═O. However, particular groups R¹ when A is a bond are optionally substituted benzyl groups such as unsubstituted benzyl, 2,6-dichlorobenzyl, 2-methoxybenzyl, and optionally substituted 1-phenylethyl groups such as 1-(phenyl)-ethyl. Examples of particular groups R¹ when A is a bond are set out in Table 1a.

In Table 1a, the point of attachment of the group to the nitrogen atom of the isothiazole-4-amino group is represented by the terminal single bond marked with an asterisk.

TABLE 1a
Examples of the group R¹ when A is a bond 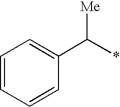
BAT 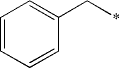
BAU 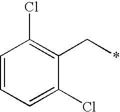
BAV 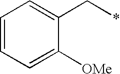
BAW 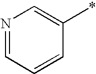
BAX 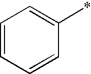
BAY

In one general embodiment, R¹ may be other than a substituted or unsubstituted tetrahydroquinoline, chroman, chromene, thiochroman, thiochromene, dihydronaphthalene or tetrahydronaphthalene group. More particularly, R¹ may be other than a substituted or unsubstituted tetrahydroquinoline, chroman, chromene, thiochroman, thiochromene, dihydro-naphthalene or tetrahydronaphthalene group linked by its aromatic ring to the moiety A-NR⁴—.

In another general embodiment, when R¹ is a substituted or unsubstituted phenyl group, the moiety Y—R³ may be other than hydrogen, unsubstituted C₁₋₁₀ alkyl, unsubstituted C₅₋₁₀ cycloalkyl, unsubstituted phenyl, unsubstituted C₁₋₁₀ alkylphenyl or unsubstituted phenyl-C₁₋₁₀ alkyl.

In the context of the group R¹-A-NR⁴—, when R¹ is an optionally substituted hydrocarbyl group and the hydrocarbyl group comprises or contains a substituted or unsubstituted alkene group, it is preferred that the carbon-carbon double bond of the alkene group is not directly bonded to the group A.

Also in the context of the group R¹-A-NR⁴—, when R¹ is an optionally substituted hydrocarbyl group, the hydrocarbyl group may be other than an alkene group.

In another general embodiment, when Y is a bond, R³ is hydrogen, A is CO and R¹ is a substituted phenyl group, each substituent on the phenyl group may be other than a group CH₂—P(O)R^{x}R^{y} where R^{x} and R^{y} are each selected from alkoxy and phenyl groups.

### Y

In the compounds of the formula (I), Y is a bond or an alkylene chain of 1, 2 or 3 carbon atoms in length.

The term “alkylene” has its usual meaning and refers to a divalent saturated acyclic hydrocarbon chain. The hydrocarbon chain may be branched or unbranched. Where an alkylene chain is branched, it may have one or more methyl group side chains. Examples of alkylene groups include —CH₂—, —CH₂—CH₂—, —CH₂—CH₂—CH₂—, CH(CH₃)—, —C(CH₃)₂—, —CH₂—CH(CH₃)—, —CH₂—C(CH₃)₂— and —CH(CH₃)—CH(CH₃)—.

In one embodiment, Y is a bond.

In another embodiment, Y is an alkylene chain.

When Y is an alkylene chain, preferably it is unbranched and more particularly contains 1 or 2 carbon atoms, preferably 1 carbon atom. Thus preferred groups Y are —CH₂— and —CH₂—CH₂—, a most preferred group being (CH₂)—.

Where Y is a branched chain, preferably it has no more than two methyl side chains. For example, it may have a single methyl side chain. In one embodiment, Y is a group —CH(Me)-.

In one sub-group of compounds, Y is a bond, CH₂, CH₂CH₂ or CH₂CH(CH₃).

### R³

The group R³ is selected from hydrogen and carbocyclic and heterocyclic groups having from 3 to 12 ring members.

In one sub-group of compounds, Y is a bond and R³ is hydrogen.

In another sub-group of compounds Y is an alkylene chain as hereinbefore defined and R³ is hydrogen.

In a another sub-group of compounds, Y is a bond or an alkylene chain (e.g. a group —(CH₂)—) and R³ is a carbocyclic or heterocyclic group.

In a further sub-group of compounds, Y is a bond and R³ is a carbocyclic or heterocyclic group.

In a still further sub-group of compounds, Y is an alkylene chain (e.g. a group —(CH₂)—) and R³ is a carbocyclic or heterocyclic group.

The carbocyclic and heterocyclic groups R³ can be aryl, heteroaryl, non-aromatic carbocyclic or non-aromatic heterocyclic and examples of such groups are as set out in detail above in the General Preferences and Definitions section, and as set out below.

Particular carbocyclic and heterocyclic groups are substituted and unsubstituted monocyclic and bicyclic groups as defined herein, with monocyclic groups being preferred.

Preferred aryl groups R³ are unsubstituted and substituted phenyl groups.

Examples of heteroaryl groups R³ include monocyclic heteroaryl groups containing up to three (and more preferably up to two) heteroatom ring members selected from O, S and N. Preferred heteroaryl groups include five membered rings containing one or two heteroatom ring members and six membered rings containing a single heteroatom ring member, most preferably nitrogen. Particular examples of heteroaryl groups include unsubstituted or substituted pyridyl, imidazole, pyrazole, thiazole, isothiazole, isoxazole, oxazole, furyl and thiophene groups.

Particular heteroaryl groups are unsubstituted and substituted pyridyl groups, e.g. 2-pyridyl, 3-pyridyl and 4-pyridyl groups, especially 3- and 4-pyridyl groups. When the pyridyl groups are substituted, they can bear one or more substituents, typically no more than two, and more usually one substituent selected, for example, from C₁₋₄ alkyl (e.g. methyl), halogen (e.g. fluorine or chlorine, preferably chlorine), and C₁₋₄ alkoxy (e.g. methoxy). Substituents on the pyridyl group may further be selected from amino, mono-C₁₋₄ alkylamino and di-C₁₋₄ alkylamino, particularly amino.

In one embodiment, when R³ is an aryl (e.g. phenyl) or heteroaryl group, the substituents on the carbocyclic or heterocyclic group may be selected from the group R^{10a} consisting of halogen, hydroxy, trifluoromethyl, cyano, monocyclic carbocyclic and heterocyclic groups having from 3 to 7 (typically 5 or 6) ring members, and a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, a carbocyclic or heterocyclic group with 3-7 ring members and a C₁₋₈ hydrocarbyl group optionally substituted by one or more substituents selected from hydroxy, oxo, halogen, cyano, nitro, carboxy, amino, mono- or di-C₁₋₄ hydrocarbylamino, a carbocyclic or heterocyclic group with 3-7 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; and R^{c}, X¹ and X² are as hereinbefore defined.

Examples of non-aromatic groups R³ include optionally substituted (by R¹⁰ or R^{10a}) cycloalkyl, oxa-cycloalkyl, aza-cycloalkyl, diaza-cycloalkyl, dioxa-cycloalkyl and aza-oxa-cycloalkyl groups. Further examples include C₇₋₁₀ aza-bicycloalkyl groups such as 1-aza-bicyclo[2.2.2]octan-3-yl.

Particular examples of such groups include unsubstituted or substituted cyclopropyl, cyclobutyl, cyclopentyl, cyclohexyl, tetrahydropyran, morpholine, tetrahydrofuran, piperidine and pyrrolidine groups.

One sub-set of non-aromatic groups R³ consists of cyclopropyl, cyclobutyl, cyclopentyl, cyclohexyl, tetrahydropyran, tetrahydrofuran, piperidine and pyrrolidine groups.

The non-aromatic groups may be unsubstituted or substituted with one or more groups R¹⁰ or R^{10a} as hereinbefore defined.

Particular substituents for R³ (e.g. (i) when R³ is an aryl or heteroaryl group or (ii) when R³ is a non-aromatic group) are selected from the group R^{10a} consisting of halogen; hydroxy; monocyclic carbocyclic and heterocyclic groups having from 3 to 6 ring members and containing up to 2 heteroatom ring members selected from O, N and S; and a group R^{a}—R^{b} wherein R^{a} is a bond, O, CO, CO₂, SO₂, NH, SO₂NH or NHSO₂; and R^{b} is selected from hydrogen, a carbocyclic or heterocyclic group with 3-6 ring members and containing up to 2 heteroatom ring members selected from O, N and S; and a C₁₋₆ hydrocarbyl group optionally substituted by one or more substituents selected from hydroxy, oxo, halogen, carboxy, amino, mono- or di-C₁₋₄ hydrocarbylamino, a carbocyclic or heterocyclic group with 3-6 ring members and containing up to 2 heteroatom ring members selected from O, N and S; and wherein one or two carbon atoms of the C₁₋₆ hydrocarbyl group may optionally be replaced by O, S, SO, SO₂ or NH.

In one embodiment, preferred R^{10a} substituent groups on R³ (e.g. (i) when R³ is an aryl or heteroaryl group or (ii) when R³ is a non-aromatic group) include halogen, a group R^{a}—R^{b} wherein R^{a} is a bond, O, CO, C(X²)X¹, and R^{b} is selected from hydrogen, heterocyclic groups having 3-7 ring members and a C₁₋₄ hydrocarbyl group optionally substituted by one or more substituents selected from hydroxy, carboxy, amino, mono- or di-C₁₋₄ hydrocarbylamino, and heterocyclic groups having 3-7 ring members.

Particularly preferred substituent groups R^{10a} on R³ (e.g. (i) when R³ is an aryl or heteroaryl group or (ii) when R³ is a non-aromatic group) include halogen, especially fluorine, C₁₋₃ alkoxy such as methoxy, and C₁₋₃ hydrocarbyl optionally substituted by fluorine, hydroxy (e.g. hydroxymethyl), C₁₋₂ alkoxy or a 5- or 6-membered saturated heterocyclic ring such as piperidino, morpholino, piperazino and N-methylpiperazino.

In another embodiment, the substituents for R³ (whether aromatic or non-aromatic) are selected from:
halogen (e.g. fluorine and chlorine)
C₁₋₄ alkoxy (e.g. methoxy and ethoxy) optionally substituted by one or substituents selected from halogen, hydroxy, C₁₋₂ alkoxy and five and six membered saturated heterocyclic rings containing 1 or 2 heteroatoms selected from O, N and S, the heterocyclic rings being optionally further substituted by one or more C₁₋₄ groups (e.g. methyl) and wherein the S, when present, may be present as S, SO or SO₂;
C₁₋₄ alkyl optionally substituted by one or substituents selected from halogen, hydroxy, C₁₋₄ alkoxy, amino, C₁₋₄ alkylsulphonylamino, 3 to 6 membered cycloalkyl groups (e.g. cyclopropyl), phenyl (optionally substituted by one or more substituents selected from halogen, methyl, methoxy and amino) and five and six membered saturated heterocyclic rings containing 1 or 2 heteroatoms selected from O, N and S, the heterocyclic rings being optionally further substituted by one or more C₁₋₄ groups (e.g. methyl) and wherein the S, when present, may be present as S, SO or SO₂;
hydroxy;
amino, mono-C₁₋₄ alkylamino, di-C₁₋₄ alkylamino, benzyloxycarbonylamino and C₁₋₄ alkoxycarbonylamino;
carboxy and C₁₋₄ alkoxycarbonyl;
C₁₋₄ alkylaminosulphonyl and C₁₋₄ alkylsulphonylamino;
C₁₋₄ alkylsulphonyl;
a group O-Het^{s} or NH-Het^{s} where Het^{s} is a five or six membered saturated heterocyclic ring containing 1 or 2 heteroatoms selected from O, N and S, the heterocyclic rings being optionally further substituted by one or more C₁₋₄ groups (e.g. methyl) and wherein the S, when present, may be present as S, SO or SO₂;
five and six membered saturated heterocyclic rings containing 1 or 2 heteroatoms selected from O, N and S, the heterocyclic rings being optionally further substituted by one or more C₁₋₄ groups (e.g. methyl) and wherein the S, when present, may be present as S, SO or SO₂;
oxo; and
six membered aryl and heteroaryl rings containing up to two nitrogen ring members and being optionally substituted by one or substituents selected from halogen, methyl and methoxy.

In one preferred sub-group of compounds, R³ is a carbocyclic or heterocyclic group R^{3a} selected from phenyl; C₃₋₆ cycloalkyl; five and six membered saturated non-aromatic heterocyclic rings containing up to two heteroatom ring members selected from N, O, S and SO₂; six membered heteroaryl rings containing one, two or three nitrogen ring members; and five membered heteroaryl rings having up to three heteroatom ring members selected from N, O and S;

wherein each carbocyclic or heterocyclic group R^{3a} is optionally substituted by up to four, preferably up to three, and more preferably up to two (e.g. one) substituents selected from amino; hydroxy; oxo; fluorine; chlorine; C₁₋₄ alkyl-(O)_{q}— wherein q is 0 or 1 and the C₁₋₄ alkyl moiety is optionally substituted by fluorine, hydroxy or C₁₋₂ alkoxy; mono-C₁₋₄ alkylamino; di-C₁₋₄ alkylamino; C₁₋₄ alkoxycarbonyl; carboxy; a group R^{e}—R¹⁶ where R^{c} is a bond or a C₁₋₃ alkylene chain and R¹⁶ is selected from C₁₋₄ alkylsulphonyl; C₁₋₄ alkylaminosulphonyl; C₁₋₄ alkylsulphonylamino-; amino; mono-C₁₋₄ alkylamino; di-C₁₋₄ alkylamino; C₁₋₇-hydrocarbyloxycarbonylamino; six membered aromatic groups containing up to three nitrogen ring members; C₃₋₆ cycloalkyl; five or six membered saturated non-aromatic heterocyclic groups containing one or two heteroatom ring members selected from N, O, S and SO₂, the group R¹⁶ when a saturated non-aromatic group being optionally substituted by one or more methyl groups, and the group R¹⁶ when aromatic being optionally substituted by one or more groups selected from fluorine, chlorine, hydroxy, C₁₋₂ alkoxy and C₁₋₂ alkyl.

In a further embodiment, R³ is selected from:
monocyclic aryl groups optionally substituted by 1-4 (for example 1-2, e.g. 1) substituents R¹⁰ or R^{10a};
C₃-C₇ cycloalkyl groups optionally substituted by 1-4 (for example 1-2, e.g. 1) substituents R¹⁰ or R^{10a};
saturated five membered heterocyclic rings containing 1 ring heteroatom selected from O, N and S and being optionally substituted by an oxo group and/or by 1-4 (for example 1-2, e.g. 1) substituents R¹⁰ or R^{10a};
saturated six membered heterocyclic rings containing 1 or 2 ring heteroatoms selected from O, N and S and being optionally substituted by an oxo group and/or by 1-4 (for example 1-2, e.g. 1) substituents R¹⁰ or R^{10a};
five membered heteroaryl rings containing 1 or 2 ring heteroatoms selected from O, N and S and being optionally substituted by 1-4 (for example 1-2, e.g. 1) substituents R¹⁰ or R^{10a};
six membered heteroaryl rings containing 1 or 2 nitrogen ring members (preferably 1 nitrogen ring member) and being optionally substituted by 1-4 (for example 1-2, e.g. 1) substituents R¹⁰ or R^{10a};
mono-azabicycloalkyl and diazabicycloalkyl groups each having 7 to 9 ring members and being optionally substituted by 1-4 (for example 1-2, e.g. 1) substituents R¹⁰ or R^{10a}.

Specific examples of the group Y—R³ are set out in Table 2. In Table 2, the point of attachment of the group to the nitrogen atom of the thiazole or isothiazole (e.g. isothiazole) carboxamide group is represented by the terminal single bond extending from the group. Thus, by way of illustration, group CA in the table is the 4-fluorophenyl, group CB in the table is the 4-methoxybenzyl group and group CC in the table is the 4-(4-methylpiperazino)-phenylmethyl group.

TABLE 2
Examples of the Group Y-R³ 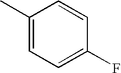
CA 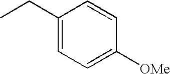
CB 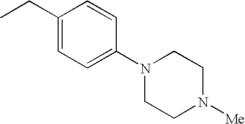
CC 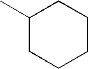
CD 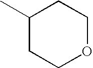
CE 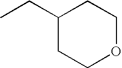
CF 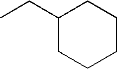
CG 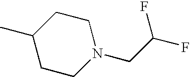
CH 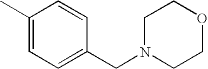
CI 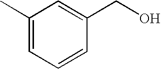
CJ 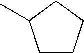
CK 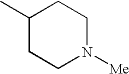
CL 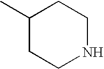
CM 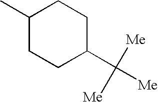
CN 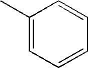
CO 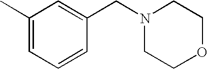
CP 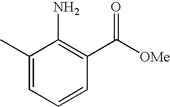
CQ 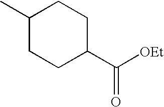
CR 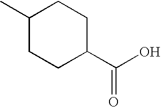
CS 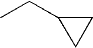
CT 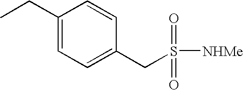
CU 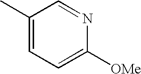
CV 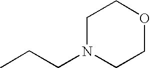
CW 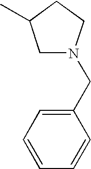
CX 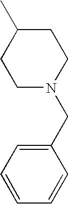
CY 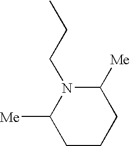
CZ 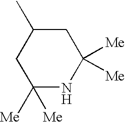
DA 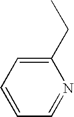
DB 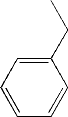
DC 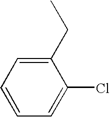
DD 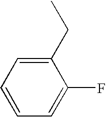
DE 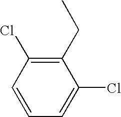
DF 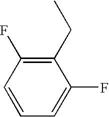
DG 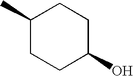
DH 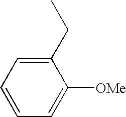
DI 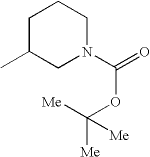
DJ 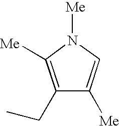
DK 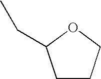
DL 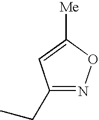
DM 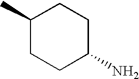
DN 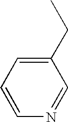
DO 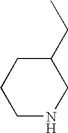
DP 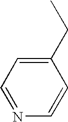
DQ 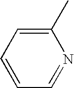
DR 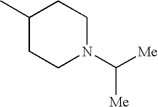
DS 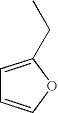
DT 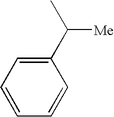
DU 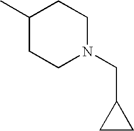
DV 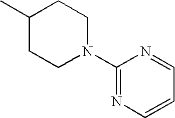
DW 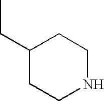
DX 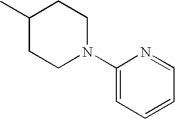
DY 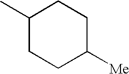
DZ 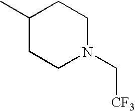
EA 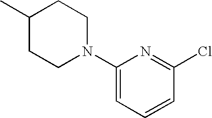
EB 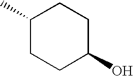
EC 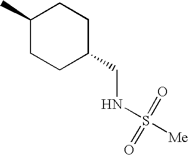
ED 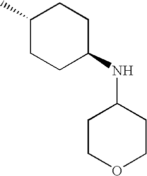
EE 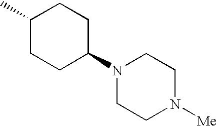
EF 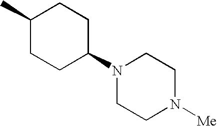
EG 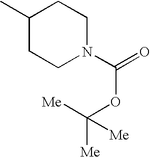
EH 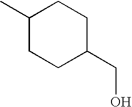
EI 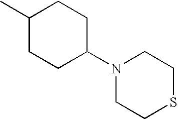
EJ 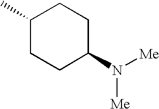
EK 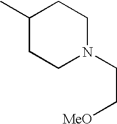
EL 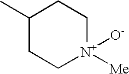
EM 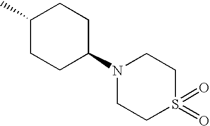
EN 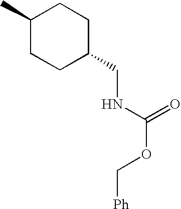
EO 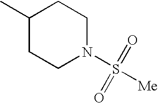
EP 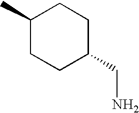
EQ 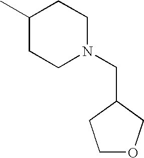
ER 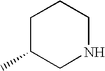
ES 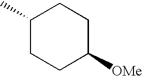
ET 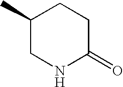
EU 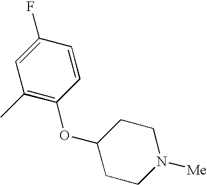
EV 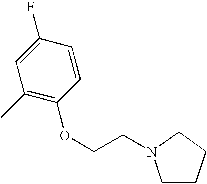
EW 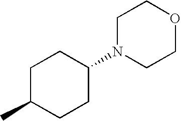
EX 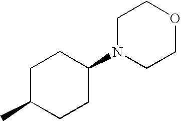
EY 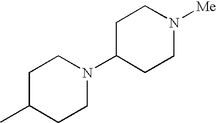
EZ 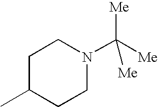
FA 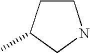
FB 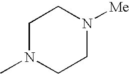
FC 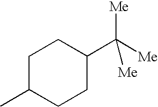
FD 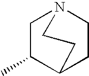
FE 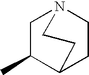
FF 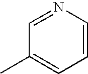
FG 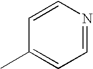
FH 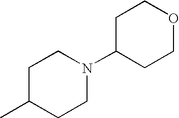
FI 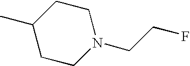
FJ 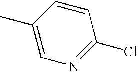
FK 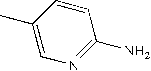
FL 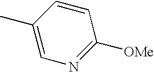
FM 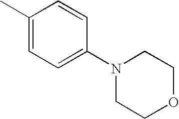
FN 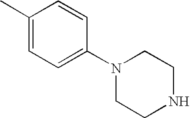
FO 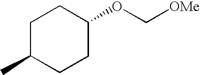
FP 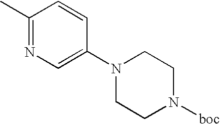
FQ 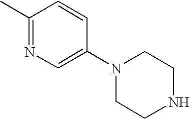
FR 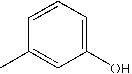
FS 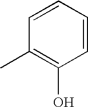
FT 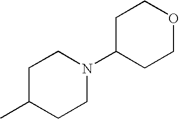
FU

One sub-set of groups selected from Table 2 consists of groups CA to FM.

Another sub-set of groups selected from Table 2 consists of groups CL, CM, CO, DR, EP, FN, FO, FP, FQ, FR, FS, FT and FU.

Preferred groups selected from Table 2 include groups CL, CM, ES, ET, FC, FG and FH.

Particularly preferred groups selected from Table 2 include groups CL, CM and ES, and most preferably CL and CM, for example CL.

Another particularly preferred group selected from Table 2 is the group ET.

Further preferred groups from Table 2 include groups FO, FR and FU.

In another general embodiment, when R³ is an aza-cycloalkyl group, A is preferably CO, NR^{g}(C═O) or O(C═O). Additionally, or alternatively, when R³ is an aza-cycloalkyl group, the nitrogen atom of the aza-cycloalkyl group is preferably not substituted with an alkylene chain linked to a 2,3-dihydro-benzo[1,4]dioxine or tetrahydronaphthalene group.

In another general embodiment, when Y is an alkylene chain of 1 carbon atom in length, R³ is other than an optionally substituted phenyl group bearing a substituted or unsubstituted cyclohexyloxy or cyclohexylthio group.

In another general embodiment, R³ is other than a moiety containing a five membered heteroaryl ring linked directly by a single bond to a monocyclic or bicyclic aryl group or R³ is other than a moiety containing a bis heteroaryl group comprising two five membered heteroaryl rings linked together by a single bond.

In a further general embodiment, R¹ is other than a moiety containing a five membered heteroaryl ring linked directly by a single bond to a monocyclic or bicyclic aryl group or R¹ is other than a moiety containing a bis heteroaryl group comprising two five membered heteroaryl rings linked together by a single bond.

In another general embodiment, R¹-A-NR⁴ is other than an optionally substituted nicotinoyl-amino or benzoyl-amino group when Y—R³ is an alkyl, cycloalkyl, optionally substituted phenyl or optionally substituted phenylalkyl group.

When A is a bond (and optionally when A is CO, NR^{g}(C═O) or O(C═O)), Y—R³ may be other than a cycloalkyl group substituted at the 1-position with a hydrocarbon chain simultaneously bearing an oxy substituent such as hydroxy, an aryl substituent and a diazole or triazole substituent.

Preferably, R¹ or R³ each are other than a moiety containing a substituted phenyl group having thio and/or oxy substituents such as hydroxy, alkoxy and alkylthio at both the 3- and 4-positions of the phenyl ring.

In a further general embodiment, when Y—R³ is unsubstituted or substituted benzyl or phenethyl or naphthylmethyl, X may be other than C₁₋₅ alkylamino or C₁₋₇ acylamino.

The group Y—R³ preferably does not include a benzo-fused lactam group having attached thereto an unsubstituted or substituted imidazole group.

The group Y—R³ preferably does not include the moiety —CH═C(CO₂R^{q})_S— where R^{q} is hydrogen or alkyl.

In another general embodiment, neither R¹ nor R³ contain a moiety in which a five membered nitrogen-containing heteroaryl group is linked directly or via an alkylene, oxa-alkylene, thia-alkylene or aza-alkylene group to an unsubstituted pyridyl group or to a substituted aryl, heteroaryl or piperidine ring, each said ring having attached thereto a substitutent selected from cyano, and substituted or unsubstituted amino, aminoalkyl, amidine, guanidine, and carbamoyl groups.

In a further general embodiment, R¹ and R³ are each other than an unsaturated nitrogen-containing heterocyclic group or a nitrogen-containing heteroaryl group, or a benzofuran or benzothiophene group wherein the said nitrogen-containing heterocyclic group, nitrogen-containing heteroaryl group, bicyclic benzofuran or benzothiophene group are linked directly by a single bond to a substituted pyridyl or phenyl group.

In another general embodiment, neither R¹ nor R³ contain a moiety in which a five membered nitrogen-containing heteroaryl group is linked directly or via an alkylene, oxa-alkylene, thia-alkylene or aza-alkylene group to a substituted aryl, heteroaryl or piperidine group or to an unsubstituted pyridyl group.

In general, it is preferred that the compounds of the invention, where they contain a carboxylic acid group, contain no more than one such group.

### Particular and Preferred Sub-Groups of the Formula (I)

One particular group of compounds of the invention is represented by the formula (II):

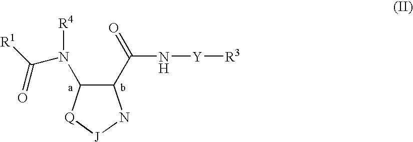

and salts, tautomers, N-oxides and solvates thereof;
wherein Q and J are as defined herein and R¹, R², R³ and Y are each independently selected from R¹, R², R³ and Y as defined herein.

Within formula (II), one sub-group of compounds has the formula (IIa):

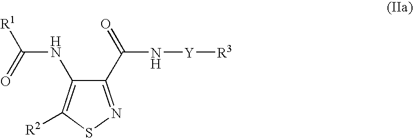

and salts, tautomers, N-oxides and solvates thereof;
wherein R¹, R², R³ and Y are each independently selected from R¹, R², R³ and Y as defined herein.

A further sub-group of compounds within formula (II) is represented by the formula (IIb):

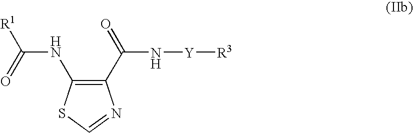

and salts, tautomers, N-oxides and solvates thereof;
wherein R¹, R³ and Y are each independently selected from R¹, R³ and Y as defined herein

Within formulae (II) and (IIa), it is preferred that R² is hydrogen or C₁₋₄ alkyl (e.g. C₁₋₃ alkyl), and more preferably R² is hydrogen.

In one sub-group of compounds of the formula (II) (or formula (IIa) or formula (IIb), R¹ is:

(i) phenyl optionally substituted by one or more substituents (e.g. 1, 2 or 3) selected from fluorine; chlorine; hydroxy; 5- and 6-membered saturated heterocyclic groups containing 1 or 2 heteroatoms selected from O, N and S, the heterocyclic groups being optionally substituted by one or more C₁₋₄ alkyl groups; C₁₋₄ hydrocarbyloxy; and C₁₋₄ hydrocarbyl; wherein the C₁₋₄ hydrocarbyl and C₁₋₄ hydrocarbyloxy groups are optionally substituted by one or more substituents chosen from hydroxy, fluorine, C₁₋₂ alkoxy, amino, mono and di-C₁₋₄ alkylamino, phenyl, halophenyl, saturated carbocyclic groups having 3 to 7 ring members (more preferably 4, 5 or 6 ring members, e.g. 5 or 6 ring members) or saturated heterocyclic groups of 5 or 6 ring members and containing up to 2 heteroatoms selected from O, S and N; or 2,3-dihydro-benzo[1,4]dioxine; or
(ii) a monocyclic heteroaryl group containing one or two heteroatoms selected from O, S and N; or a bicyclic heteroaryl group containing a single heteroatom selected from O, S and N; the monocyclic and bicyclic heteroaryl groups each being optionally substituted by one or more substituents selected from fluorine; chlorine; C₁₋₃ hydrocarbyloxy; and C₁₋₃ hydrocarbyl optionally substituted by hydroxy, fluorine, methoxy or a five or six membered saturated carbocyclic or heterocyclic group containing up to two heteroatoms selected from O, S and N; or
(iii) a substituted or unsubstituted cycloalkyl group having from 3 to 6 ring members; or
(iv) a C₁₋₄ hydrocarbyl group optionally substituted by one or more substituents selected from fluorine; hydroxy; C₁₋₄ hydrocarbyloxy; amino; mono- or di-C₁₋₄ hydrocarbylamino; and carbocyclic or heterocyclic groups having from 3 to 12 ring members, and wherein one of the carbon atoms of the hydrocarbyl group may optionally be replaced by an atom or group selected from O, NH, SO and SO₂.

In a further alternative, R¹ can be:

(v) a non-aromatic heterocyclic group selected from pyrrolidine, piperidine, tetrahydropyran, tetrahydrothiopyran, morpholine, thiomorpholine, thiomorpholine S,S-dioxide, piperazine, N-alkyl piperazines, and N-alkyl piperidines.

Within the group of compounds defined by the formula (II) (or formulae (Ia) and (IIb)), where R¹ is (i) an optionally substituted phenyl group, it may be, for example, an unsubstituted phenyl group or a 2-monosubstituted, 3-monosubstituted, 2,3 disubstituted, 2,5 disubstituted or 2,6 disubstituted phenyl group or 2,3-dihydro-benzo[1,4]dioxine, where the substituents are selected from halogen; hydroxyl; C₁₋₃ alkoxy; and C₁₋₃ alkyl groups wherein the C₁₋₃ alkyl group is optionally substituted by hydroxy, fluorine, C₁₋₂ alkoxy, amino, mono and di-C₁₋₄ alkylamino, or saturated carbocyclic groups having 3 to 6 ring members and/or saturated heterocyclic groups of 5 or 6 ring members and containing 1 or 2 heteroatoms selected from N and O.

In a further embodiment within the group of compounds defined by the formula (II) (or formulae (IIa) and (IIb)), where R¹ is (i) an optionally substituted phenyl group, it may be, for example, a 2,4,6-trisubstituted phenyl group or a 2,3,6-trisubstituted phenyl group.

In one embodiment, R¹ is selected from unsubstituted phenyl, 2-fluorophenyl, 2-hydroxyphenyl, 2-methoxyphenyl, 2-methylphenyl, 2-(2-(pyrrolidin-1-yl)ethoxy)-phenyl, 3-fluorophenyl, 3-methoxyphenyl, 2,6-difluorophenyl, 2-fluoro-6-hydroxyphenyl, 2-fluoro-3-methoxyphenyl, 2-fluoro-5-methoxyphenyl, 2-chloro-6-methoxyphenyl, 2-fluoro-6-methoxyphenyl, 2,6-dichlorophenyl, 2-chloro-6-fluorophenyl, 5-fluoro-2-methoxyphenyl, 2-methoxy-4-chlorophenyl, and 2,3-dihydro-benzo[1,4]dioxin-5-yl.

In one sub-group of compounds, R¹ is selected from unsubstituted phenyl, 2-fluorophenyl, 2-hydroxyphenyl, 2-methoxyphenyl, 2-methylphenyl, 2-(2-(pyrrolidin-1-yl)ethoxy)-phenyl, 3-fluorophenyl, 3-methoxyphenyl, 2,6-difluorophenyl, 2-fluoro-6-hydroxyphenyl, 2-fluoro-3-methoxyphenyl, 2-fluoro-5-methoxyphenyl, 2-chloro-6-methoxyphenyl, 2-fluoro-6-methoxyphenyl, 2,6-dichlorophenyl, 2-chloro-6-fluorophenyl and 5-fluoro-2-methoxyphenyl.

Particular groups R¹ are 2,6-difluorophenyl, 2-fluoro-6-methoxyphenyl and 2,6-dichlorophenyl.

One particularly preferred group R¹ is 2,6-difluorophenyl.

Another particularly preferred group R¹ is 2,6-dichlorophenyl.

In another embodiment, R¹ is 2,4,6-trichlorophenyl.

When R¹ is (ii) a monocyclic heteroaryl group containing one or two heteroatoms selected from O, S and N or a bicyclic heteroaryl group containing a single heteroatom, examples of monocyclic and bicyclic heteroaryl groups include furanyl (e.g. 2-furanyl and 3-furanyl), imidazolyl, pyridyl (e.g. 2-pyridyl), indolyl, thienyl (e.g. 2-thienyl and 3-thienyl) groups. The optional substituents for such groups can include chlorine, fluorine, methyl, methoxy, hydroxymethyl, methoxymethyl, morpholinomethyl, piperazinomethyl, N-methylypiperazinomethyl and piperidinylmethyl groups. Particular examples of groups (ii) include unsubstituted 2-furanyl, 3-methyl-2-furanyl, unsubstituted 4-(1H)-imidazolyl, unsubstituted 5-(1H)-imidazolyl, unsubstituted 3-furanyl, unsubstituted 3-thienyl, 2-methyl-3-thienyl and unsubstituted 3-pyrrolyl, and further examples include 4-methoxy-3-thienyl, 5-(1-pyrrolidinyl)methyl-2-furyl and 5-(4-morpholino)methyl-2-furyl groups.

When R¹ is (iii) an optionally substituted cycloalkyl group, it can be for example a substituted or unsubstituted cyclopropyl, cyclobutyl, cyclopentyl or cyclohexyl group. When the cycloalkyl group is substituted, preferred substituents include methyl, fluorine and hydroxyl. Particular examples of cycloalkyl groups include 1-methylcyclopropyl, 1-hydroxycyclopropyl, and unsubstituted cyclohexyl, cyclopentyl and cyclobutyl.

In another embodiment, R¹ is a saturated 5- or 6-membered heterocyclic ring containing one or two heteroatom ring members selected from O, N and S. The saturated heterocyclic ring may be unsubstituted or substituted by one or more substitutents R¹⁰ as defined herein.

Examples of saturated 5- or 6-membered heterocyclic rings are set out above in the General Preferences and Definitions section.

Particular examples include piperidine (such as 4-piperidine) and N-substituted forms thereof such as N-methyl piperidine, piperazine) and N-substituted forms thereof such as N-methyl piperazine, pyrrolidine, tetrahydropyran, morpholine and thiomorpholine and the S-oxide and S,S-dioxide thereof.

Specific examples of groups R¹—CO— in formula (II) are set out in Table 1 above.

Preferred groups R1 include the groups AJ, BQ and BS in Table 1, more particularly the sub-set consisting of AJ and BQ.

Another group of compounds of the invention is represented by the formula (III):

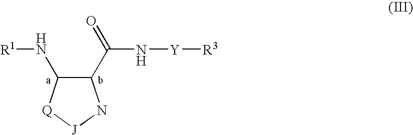

and salts, tautomers, N-oxides and solvates thereof;
wherein Q, J, the ring carbon atoms “a” and “b”, R¹, R³ and Y are as defined herein;

but excluding the compound wherein the ring containing the moiety Q-J is a thiazole ring, R¹ is cyclohexyl, Y is a bond and R³ is a methoxy-substituted dibenzofuran group.

Within formula (III), one sub-group of compounds is represented by the formula (IIIa):

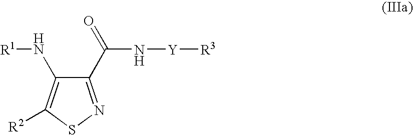

and salts, tautomers, N-oxides and solvates thereof;
wherein R¹, R², R³ and Y are as defined herein.

Another sub-group of compounds within formula (III) is represented by formula (IIIb):

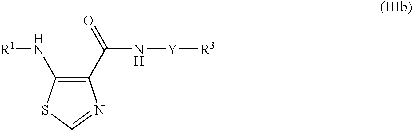

and salts, tautomers, N-oxides and solvates thereof;
wherein R¹, R³ and Y are as defined herein;

but excluding the compound wherein R¹ is cyclohexyl, Y is a bond and R³ is a methoxy-substituted dibenzofuran group.

Examples of, and preferences, for the groups R¹, R², R³ and Y are as set out above for compounds of the formulae (I) to (Ie), (II), (IIa) and (IIb) unless the context indicates otherwise.

Particular sub-groups of compounds of the formulae (III), (IIa) and (IIIb) include:

(i) compounds wherein R¹ is a heteroaryl group containing 1, 2 or 3 heteroatom ring members selected from N, O and S;
(ii) compounds wherein R¹ is a C₁₋₄ hydrocarbyl group optionally substituted by a carbocyclic or heterocyclic group having from 3 to 12 ring members; and
(iii) compounds wherein R¹ is a non-aromatic carbocyclic or heterocyclic group having from 3 to 12 ring members.

A further sub-group of compounds of the formulae (III), (IIIa) and (IIIb) includes:

(iv) compounds wherein R¹ is an aryl group

A still further sub-group of the formulae (III), (IIIa) and (IIIb) includes:

(v) compounds wherein R¹ is an arylalkyl or heteroarylalkyl group wherein the aryl and heteroaryl moieties are optionally substituted monocyclic groups, for example substituted and unsubstituted benzyl, phenyl-1-ethyl and pyridylmethyl groups.

Examples of compounds of the formula (III) (or (IIIa) or (IIIb)) wherein R¹ is (i) a heteroaryl group include 5- and 6-membered monocyclic heteroaryl groups, e.g. containing 1 or 2 heteroatom ring members selected from O, N and S. In one embodiment, the heteroaryl group is a monocyclic group containing 1 or 2 nitrogen ring members. In another embodiment, the heteroaryl groups are selected from 6-membered rings containing 1 or 2 nitrogen ring members, for example pyridine, pyrimidine, pyrazine and pyridazine groups, one particular sub-group consisting of pyrazinyl and pyridyl.

The heteroaryl groups can be unsubstituted or substituted by one or more groups R¹⁰ as defined herein.

Examples of compounds of the formulae (III), (IIIa) and (IIIb) wherein R¹ is (ii) an optionally substituted C₁₋₄ hydrocarbyl group include those in which the hydrocarbyl group is unsubstituted hydrocarbyl, for example unsubstituted alkyl such as methyl, ethyl, propyl, isopropyl, butyl, isobutyl and t-butyl.

Further examples of compounds of the formulae (III), (IIIa) and (IIIb) wherein R¹ is (ii) an optionally substituted C₁₋₄ hydrocarbyl group include those in which the hydrocarbyl group is substituted alkyl such as substituted methyl or ethyl, wherein the substituent is an aryl or heteroaryl group optionally substituted by one or more substituent groups R¹⁰ as defined herein. Particular examples of aryl and heteroaryl groups are monocyclic groups such as optionally substituted phenyl. More particularly, R¹ may be an optionally substituted benzyl or α-methylbenzyl in which the phenyl group is optionally substituted.

Examples of compounds wherein R¹ is a non-aromatic carbocyclic or heterocyclic group include those wherein the carbocyclic or heterocylic group is monocyclic and contains up to 2 heteroatoms selected from oxygen and nitrogen. Particular examples of such groups are cyclohexyl and piperidine. Further particular examples include tetrahydropyranyl, morpholinyl, pyrrolidinyl and N-methylpiperidinyl.

Examples of compounds of the formula (III) (or (IIIa) or (IIIb)) wherein R¹ is (iv) an aryl group include those wherein R¹ is a phenyl group which may be unsubstituted or substituted as defined herein.

Another sub-group of compounds of the formula (I) can be represented by the formula (IV):

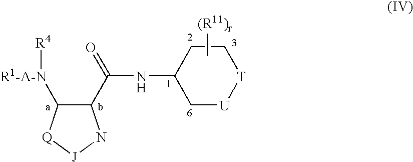

and salts, tautomers, N-oxides and solvates thereof;
wherein A, J, Q, R¹ and R⁴ are as defined herein;
an optional second bond may be present between carbon atoms numbered 1 and 2; one of U and T is selected from CH₂, CHR¹³, CR¹¹R³, NR¹⁴, N(O)R¹⁵, O and S(O)ₜ; and the other of U and T is selected from, NR¹⁴, O, CH₂, CHR¹¹, C(R¹¹)₂, and C═O; r is 0, 1, 2, 3 or 4; t is 0, 1 or 2;
R¹¹ is selected from hydrogen, halogen (particularly fluorine), C₁₋₃ alkyl (e.g. methyl) and C₁₋₃ alkoxy (e.g. methoxy);
R¹³ is selected from hydrogen, NHR¹⁴, NOH, NOR¹⁴ and R^{a}—R^{b};
R¹⁴ is selected from hydrogen and R^{d}—R^{b};
R^{d} is selected from a bond, CO, C(X²)X¹, SO₂ and SO₂NR^{c};
R^{a}, R^{b} and R^{c} are as hereinbefore defined; and R¹⁵ is selected from C₁₋₄ saturated hydrocarbyl optionally substituted by hydroxy, C₁₋₂ alkoxy, halogen or a monocyclic 5- or 6-membered carbocyclic or heterocyclic group, provided that U and T cannot be 0 simultaneously.

In one sub-group of compounds within formula (IV), the moiety:

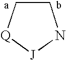

is an isothiazole group.

In another sub-group of compounds within formula (IV), the moiety

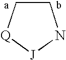

is a thiazole group.

In respect of formula (IV) and the isothiazole and thiazole sub-groups thereof as defined above, examples of, and preferences, for the groups R¹ and R² are as set out above for compounds of the formulae (I) to (Ie), (II), (Ia), (IIb), (III), (IIa) and (IIIb) unless the context indicates otherwise.

Within formula (IV), r can be 0, 1, 2, 3 or 4. In one embodiment, r is 0. In another embodiment, r is 2, and in a further embodiment r is 4.

Within formula (IV), one sub-set of preferred compounds is the set of compounds where there is only a single bond between the carbon atoms numbered 1 and 2.

However, in another sub-set of compounds, there is a double bond between the carbon atoms numbered 1 and 2.

Another sub-set of compounds is characterised by gem disubstitution at the 2-carbon (when there is a single bond between carbon atoms numbers 1 and 2) and/or the 6-carbon. Preferred gem disubstituents include difluoro and dimethyl.

A further sub-set of compounds is characterised by the presence of an alkoxy group, for example a methoxy group at the carbon atom numbered 3, i.e. at a position α with respect to the group T.

Within formula (IV) are compounds wherein, for example, R³ is selected from any of the following ring systems:

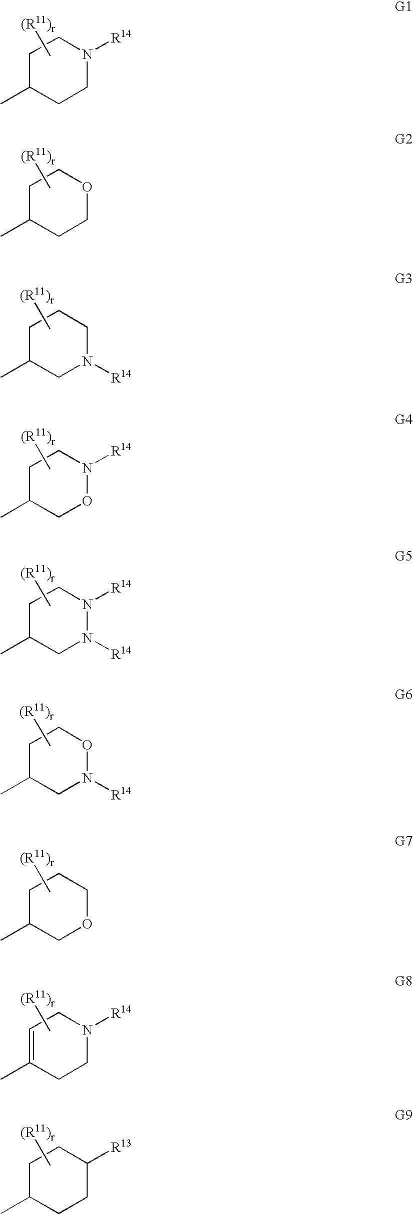

Preferred ring systems include G1 and G3. Another preferred ring system is G9.

In a preferred sub-group of compounds within formula (IV), one of U and T is selected from CH₂, CHR¹³, CR¹¹R³, NR¹⁴, N(O)R¹⁵, O and S(O)ₜ; and the other of U and T is selected from CH₂, CHR¹¹, C(R¹¹)₂, and C═O; r is 0, 1 or 2; t is 0, 1 or 2;

R¹¹ is selected from hydrogen and C₁₋₃ alkyl;
R¹³ is selected from hydrogen and R^{a}—R^{b};
R¹⁴ is selected from hydrogen and R^{d}—R^{b};
R^{d} is selected from a bond, CO, C(X²)X¹, SO₂ and SO₂NR^{c};
R^{a}, R^{b} and R^{c} are as hereinbefore defined; and
R¹⁵ is selected from C₁₋₄ saturated hydrocarbyl optionally substituted by hydroxy, C₁₋₂ alkoxy, halogen or a monocyclic 5- or 6-membered carbocyclic or heterocyclic group.

In one embodiment, T is selected from CH₂, CHR¹³, CR¹¹R¹³, NR¹⁴, N(O)R⁵, O and S(O)ₜ; and U is preferably selected from CH₂, CHR¹¹, C(R¹¹)₂, and C═O.

In the definitions for substituents R¹¹ and R¹⁴, R^{b} is preferably selected from hydrogen; monocyclic carbocyclic and heterocyclic groups having from 3 to 7 ring members; and C₁₋₄ hydrocarbyl (more preferably acyclic saturated C₁₋₄ groups) optionally substituted by one or more substituents selected from hydroxy, oxo, halogen, amino, mono- or di-C₁₋₄ hydrocarbylamino, and monocyclic carbocyclic and heterocyclic groups having from 3 to 7 ring members (more preferably 3 to 6 ring members) and wherein one or more carbon atoms of the C₁₋₄ hydrocarbyl group may optionally be replaced by O, S, SO, SO₂, NR^{c}, X¹C(X²), C(X²)X¹; R^{c} is selected from hydrogen and C₁₋₄ hydrocarbyl; and

X¹ is O, S or NR^{c} and X² is ═O, ═S or ═NR^{c}.

R¹¹ is preferably selected from hydrogen and methyl and most preferably is hydrogen.

R¹³ is preferably selected from hydrogen; hydroxy; halogen; cyano; amino; mono-C₁₋₄ saturated hydrocarbylamino; di-C₁₋₄ saturated hydrocarbylamino; monocyclic 5- or 6-membered carbocyclic and heterocyclic groups; C₁₋₄ saturated hydrocarbyl optionally substituted by hydroxy, C₁₋₂ alkoxy, halogen or a monocyclic 5- or 6-membered carbocyclic or heterocyclic group.

Particular examples of R¹³ are hydrogen, hydroxy, amino, C₁₋₂ alkylamino (e.g. methylamino) C₁₋₄ alkyl (e.g. methyl, ethyl, propyl and butyl), C₁₋₂ alkoxy (e.g. methoxy), C₁₋₂ alkylsulphonamido (e.g. methanesulphonamido), hydroxy-C₁₋₂ alkyl (e.g. hydroxymethyl), C₁₋₂-alkoxy-C₁₋₂ alkyl (e.g. methoxymethyl and methoxyethyl), carboxy, C₁₋₄ alkoxycarbonyl (e.g. ethoxycarbonyl) and amino-C₁₋₂-alkyl (e.g. aminomethyl).

One preferred example of R³, particularly in the context of the group G9, is a methoxy group.

Particular examples of R¹⁴ are hydrogen; C₁₋₄ alkyl optionally substituted by fluoro or a five or six membered saturated heterocyclic group (e.g. a group selected from (i) methyl, ethyl, n-propyl, i-propyl, butyl, 2,2,2-trifluoroethyl and tetrahydrofuranylmethyl; and/or (ii) 2-fluoroethyl and 2,2-difluoroethyl); cyclopropylmethyl; substituted or unsubstituted pyridyl-C₁₋₂ alkyl (e.g. 2-pyridylmethyl); substituted or unsubstituted phenyl-C₁₋₂ alkyl (e.g. benzyl); C₁₋₄ alkoxycarbonyl (e.g. ethoxycarbonyl and t-butyloxycarbonyl); substituted and unsubstituted phenyl-C₁₋₂ alkoxycarbonyl (e.g. benzyloxycarbonyl); substituted and unsubstituted 5- and 6-membered heteroaryl groups such as pyridyl (e.g. 2-pyridyl and 6-chloro-2-pyridyl) and pyrimidinyl (e.g. 2-pyrimidinyl); C₁₋₂-alkoxy-C₁₋₂ alkyl (e.g. methoxymethyl and methoxyethyl); C₁₋₄ alkylsulphonyl (e.g. methanesulphonyl).

Preferred compounds include those in which (i) U is CHR¹³ (more preferably CH₂) and T is NR¹⁴, and (ii) T is CHR¹³ (more preferably CH₂) and U is NR¹⁴.

One preferred sub-group of compounds of the formula (IV) can be represented by the formula (V):

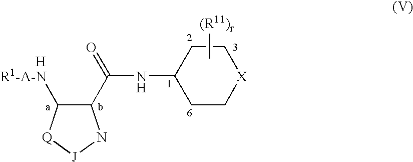

and salts, tautomers, N-oxides and solvates thereof;
wherein Q, J, the ring carbon atoms “a” and “b”, R¹, R¹¹ and “r” are as defined herein and X is selected from CH-OMe and a group NR¹⁴ as defined herein.

In one sub-group of compounds within formula (V), the moiety:

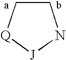

is an isothiazole group.

In another sub-group of compounds within formula (V), the moiety

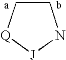

is a thiazole group.

Particular groups within formula (V) are thiazoles and isothiazoles wherein X is a CHOMe, NH or NMe group and r is 0.

One particular preferred sub-group of compounds of the formula (V) can be represented by the formula (Va):

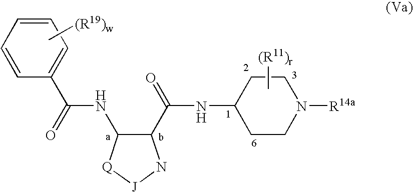

and salts, tautomers, N-oxides and solvates thereof;
wherein R^{14a} is selected from hydrogen, C₁₋₄ alkyl optionally substituted by fluoro (e.g. methyl, ethyl, n-propyl, i-propyl, butyl, 2-fluoroethyl, 2,2-difluoroethyl and 2,2,2-trifluoroethyl), cyclopropylmethyl, phenyl-C₁₋₂ alkyl (e.g. benzyl), C₁₋₄ alkoxycarbonyl (e.g. ethoxycarbonyl and t-butyloxycarbonyl), phenyl-C₁₋₂ alkoxycarbonyl (e.g. benzyloxycarbonyl), C₁₋₂-alkoxy-C₁₋₂ alkyl (e.g. methoxymethyl and methoxyethyl), and C₁₋₄ alkylsulphonyl (e.g. methanesulphonyl), wherein the phenyl moieties when present are optionally substituted by one to three substituents selected from fluorine, chlorine, C₁₋₄ alkoxy optionally substituted by fluoro or C₁₋₂-alkoxy, and C₁₋₄ alkyl optionally substituted by fluoro or C₁₋₂-alkoxy;
w is 0, 1, 2 or 3;
R² is hydrogen or methyl, most preferably hydrogen;
R¹¹ and r are as hereinbefore defined; and
R¹⁹ is selected from fluorine; chlorine; C₁₋₄ alkoxy optionally substituted by fluoro or C₁₋₂-alkoxy; and C₁₋₄ alkyl optionally substituted by fluoro or C₁₋₂-alkoxy.

Another particular preferred sub-group of compounds of the formula (IV) can be represented by the formula (Vb):

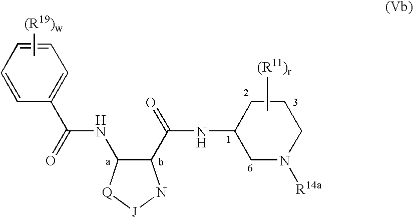

and salts, tautomers, N-oxides and solvates thereof;
wherein R^{14a} is selected from hydrogen, C₁₋₄ alkyl optionally substituted by fluoro (e.g. methyl, ethyl, n-propyl, i-propyl, butyl and 2,2,2-trifluoroethyl), cyclopropylmethyl, phenyl-C₁₋₂ alkyl (e.g. benzyl), C₁₋₄ alkoxycarbonyl (e.g. ethoxycarbonyl and t-butyloxycarbonyl), phenyl-C₁₋₂ alkoxycarbonyl (e.g. benzyloxycarbonyl), C₁₋₂-alkoxy-C₁₋₂ alkyl (e.g. methoxymethyl and methoxyethyl), and C₁₋₄ alkylsulphonyl (e.g. methanesulphonyl), wherein the phenyl moieties when present are optionally substituted by one to three substituents selected from fluorine, chlorine, C₁₋₄ alkoxy optionally substituted by fluoro or C₁₋₂-alkoxy, and C₁₋₄ alkyl optionally substituted by fluoro or C₁₋₂-alkoxy;
w is 0, 1, 2 or 3;
R² is hydrogen or methyl, most preferably hydrogen;
R¹¹ and r are as hereinbefore defined; and
R¹⁹ is selected from fluorine; chlorine; C₁₋₄ alkoxy optionally substituted by fluoro or C₁₋₂-alkoxy; and C₁₋₄ alkyl optionally substituted by fluoro or C₁₋₂-alkoxy.

In formulae (Va) and (Vb), when w is 1, 2 or 3, it is preferred that the phenyl ring is 2-monosubstituted, 3-monosubstituted, 2,6-disubstituted, 2,3-disubstituted, 2,4-disubstituted 2,5-disubstituted, 2,3,6-trisubstituted or 2,4,6-trisubstituted. Most preferably the phenyl ring is disubstituted at positions 2- and 6- with substituents selected from fluorine, chlorine and methoxy.

R¹¹ is preferably hydrogen (or r is 0).

R^{14a} is most preferably hydrogen or methyl.

One preferred sub-group of compounds of the formula (Va) can be represented by the formula (VI):

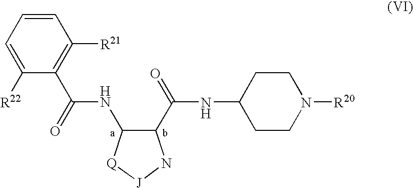

and salts, tautomers, N-oxides and solvates thereof;
wherein R²⁰ is selected from hydrogen and methyl;
R²¹ is selected from fluorine and chlorine; and
R²² is selected from fluorine, chlorine and methoxy; or
one of R²¹ and R²² is hydrogen and the other is selected from chlorine, methoxy, ethoxy, difluoromethoxy, trifluoromethoxy and benzyloxy.

Within formula (VI), particular compounds are those wherein R²⁰ is selected from hydrogen and methyl; R²¹ is selected from fluorine and chlorine; and R²² is selected from fluorine, chlorine and methoxy.

Within formula (V), a further group of preferred compounds can be represented by the formula (VII):

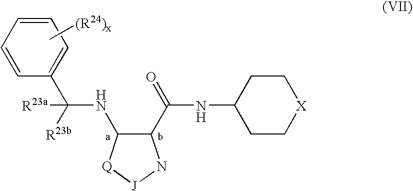

and salts, solvates, tautomers and N-oxides thereof;
wherein Q, J and X are as defined herein, x is 0, 1, 2 or 3, R^{23a} and R^{23b} are each independently selected from hydrogen, methyl and fluorine, and R²⁴ is selected from methyl, ethyl, methoxy, ethoxy, chlorine and fluorine.

Another sub-group of compounds within formula (I) is represented by formula (VIII):

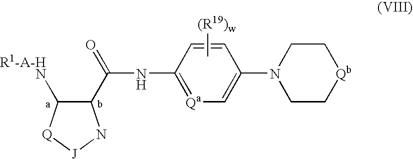

wherein R¹, A, Q, J, R¹⁹, and w are as hereinbefore defined, Q^{a} is N, CH or CR¹⁹ and Q^{b} is selected from O, NH, N—(C₁₋₄ alkyl) and N—C(O)—O(C₁₋₄ alkyl).

In one sub-group of compounds within formula (VIII), Q^{a} is N.

In another sub-group of compounds within formula (VIII), Q^{a} is CH.

Particular examples of Q^{b} are O, NH, N-Me and N-boc.

In each of sub-groups (Va), (Vb), (VI), (VII) and (VIII), the moiety:

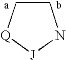

can be a thiazole group or an isothiazole group. In one embodiment, the said moiety is a thiazole group. In another embodiment, the said moiety is an isothiazole group.

For the avoidance of doubt, it is to be understood that each general and specific preference, embodiment and example of the groups R¹ may be combined with each general and specific preference, embodiment and example of the groups R² and/or R³ and/or R⁴ and/or R¹⁰ and/or Y and/or R^{g} and/or sub-groups thereof as defined herein and that all such combinations are embraced by this application.

The various functional groups and substituents making up the compounds of the formulae (I), (Ia), (Ib), (Ic), (Id), (Ie), (II), (IIa), (IIb), (III), (IIIa), (IIIb), (IV), (V), (Va), (Vb), (VI), (VIa), (VII) and (VIII) are typically chosen such that the molecular weight of the compound of the formula and sub-groups thereof does not exceed 1000. More usually, the molecular weight of the compound will be less than 750, for example less than 700, or less than 650, or less than 600, or less than 550. More preferably, the molecular weight is less than 525 and, for example, is 500 or less.

Particular compounds of the invention are as illustrated in the examples below and include:
4-(2,6-dichloro-benzoylamino)-isothiazole-3-carboxylic acid (1-methyl-piperidin-4-yl)-amide;
5-(2,6-difluoro-benzoylamino)-thiazole-4-carboxylic acid piperidin-4-ylamide;
5-benzylamino-thiazole-4-carboxylic acid piperidin-4-ylamide;
5-(2,6-dichloro-benzylamino)-thiazole-4-carboxylic acid piperidin-4-ylamide;
5-(2-ethoxy-benzoylamino)-thiazole-4-carboxylic acid piperidin-4-ylamide;
5-(1-phenyl-ethylamino)-thiazole-4-carboxylic acid piperidin-4-ylamide;
5-(2-methoxy-benzylamino)-thiazole-4-carboxylic acid piperidin-4-ylamide; and
5-(pyridin-3-ylamino)-thiazole-4-carboxylic acid piperidin-4-ylamide;
4-(2,6-dichloro-benzoylamino)-isothiazole-3-carboxylic acid (4-piperazin-1-yl-phenyl)-amide;
5-(2,4,6-trichloro-benzoylamino)-thiazole-4-carboxylic acid phenylamide;
5-(2,4,6-trichloro-benzoylamino)-thiazole-4-carboxylic acid (5-piperazin-1-yl-pyridin-2-yl)-amide;
5-(2,4,6-trichloro-benzoylamino)-thiazole-4-carboxylic acid [1-(tetrahydro-pyran-4-yl)-piperidin-4-yl]-amide;
5-(2,4,6-trichloro-benzoylamino)-thiazole-4-carboxylic acid pyridin-2-yl amide;
5-(2,4,6-trichloro-benzoylamino)-thiazole-4-carboxylic acid piperidin-4-yl amide;
5-(2,4,6-trichloro-benzoylamino)-thiazole-4-carboxylic acid (4-morpholin-4-yl-phenyl)-amide;
5-(2,4,6-trichloro-benzoylamino)-thiazole-4-carboxylic acid (4-methoxy methoxy-cyclohexyl)-amide;
5-(2,4,6-trichloro-benzoylamino)-thiazole-4-carboxylic acid (4-piperazin-1-yl-phenyl)-amide;
5-(2,4,6-trichloro-benzoylamino)-thiazole-4-carboxylic acid (3-hydroxy-phenyl)-amide;
5-(2,4,6-trichloro-benzoylamino)-thiazole-4-carboxylic acid (2-hydroxy-phenyl)-amide;
5-phenylamino-thiazole-4-carboxylic acid phenylamide;
5-phenylamino-thiazole-4-carboxylic acid pyridin-2-yl-amide;
5-phenylamino-thiazole-4-carboxylic acid (5-piperazin-1-yl-pyridin-2-yl)-amide;
5-phenylamino-thiazole-4-carboxylic acid (4-piperazin-1-yl-phenyl)-amide;
5-(2-methoxy-benzylamino)-thiazole-4-carboxylic acid (5-piperazin-1-yl-pyridin-2-yl)-amide;
4-(2,6-dichloro-benzoylamino)-isothiazole-3-carboxylic acid (5-piperazin-1-yl-pyridin-2-yl)-amide;
4-(2,6-dichloro-benzoylamino)-isothiazole-3-carboxylic acid (1-methanesulfonyl-piperidin-4-yl)-amide;
4-benzoylamino-isothiazole-3-carboxylic acid (4-piperazin-1-yl-phenyl)-amide;
4-(cyclopentane-carbonyl-amino)-isothiazole-3-carboxylic acid (5-piperazin-1-yl-pyridin-2-yl)-amide;
4-(2,4,6-trichloro-benzoylamino)-isothiazole-3-carboxylic acid (5-piperazin-1-yl-pyridin-2-yl)-amide;
5-(2,6-dichloro-benzoylamino)-thiazole-4-carboxylic acid (4-piperazin-1-yl-phenyl)-amide; and
5-benzoylamino-thiazole-4-carboxylic acid (4-piperazin-1-yl-phenyl)-amide; and salts, tautomers, N-oxides and solvates thereof.

### Salts, Solvates, Tautomers, Isomers, N-Oxides, Esters, Prodrugs and Isotypes

A reference to a compound of the formulae (I) and sub-groups thereof also includes ionic forms, salts, solvates, isomers, tautomers, N-oxides, esters, prodrugs, isotopes and protected forms thereof, for example, as discussed below; preferably, the salts or tautomers or isomers or N-oxides or solvates thereof; and more preferably, the salts or tautomers or N-oxides or solvates thereof.

For example, a reference to a particular compound also includes ionic, salt, solvate, and protected forms thereof, for example, as discussed below.

Many compounds of the formula (I) can exist in the form of salts, for example acid addition salts or, in certain cases salts of organic and inorganic bases such as carboxylate, sulphonate and phosphate salts. All such salts are within the scope of this invention, and references to compounds of the formula (I) include the salt forms of the compounds. As in the preceding sections of this application, all references to formula (I) should be taken to refer also to formulae (Ia), (Ib), (Ic), (Id), (Ie), (II), (IIa), (IIb), (III), (IIIa), (IIIb), (IV), (V), (Va), (Vb), (VI), (VIa) or (VII) and sub-groups thereof unless the context indicates otherwise.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two; generally, nonaqueous media such as ether, ethyl acetate, ethanol, isopropanol, or acetonitrile are used.

Acid addition salts may be formed with a wide variety of acids, both inorganic and organic. Examples of acid addition salts include salts formed with an acid selected from the group consisting of acetic, 2,2-dichloroacetic, adipic, alginic, ascorbic (e.g. L-ascorbic), L-aspartic, benzenesulphonic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrobromic, hydrochloric, hydriodic, isethionic, (+)-L-lactic, (±)-DL-lactic, lactobionic, maleic, malic, (−)-L-malic, malonic, (±)-DL-mandelic, methanesulphonic, naphthalene-2-sulphonic, naphthalene-1,5-disulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, (+)-L-tartaric, thiocyanic, p-toluenesulphonic, undecylenic and valeric acids, as well as acylated amino acids and cation exchange resins.

One particular group of salts consists of salts formed from hydrochloric, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, acetic, propanoic, butanoic, malonic, glucuronic and lactobionic acids.

One preferred group of salts consists of salts formed from hydrochloric, acetic, adipic, L-aspartic and DL-lactic acids.

Particularly preferred salts are hydrochloride salts

For example, if the compound is anionic, or has a functional group which may be anionic (e.g., —COOH may be —COO—), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the formula (I) contain an amine function, these may form quaternary ammonium salts, for example by reaction with an alkylating agent according to methods well known to the skilled person. Such quaternary ammonium compounds are within the scope of formula (I).

In one embodiment, the salt of the compound of formula (I) is other than a quaternary ammonium salt.

The salt forms of the compounds of the invention are typically pharmaceutically acceptable salts, and examples of pharmaceutically acceptable salts are discussed in Berge et al., 1977, “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention.

Compounds of the formula (I) containing an amine function may also form N-oxides. A reference herein to a compound of the formula (I) that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Compounds of the formula (I) may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

Examples of tautomeric forms include, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

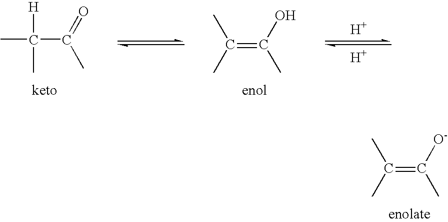

Where compounds of the formula (I) contain one or more chiral centres, and can exist in the form of two or more optical isomers, references to compounds of the formula (I) include all optical isomeric forms thereof (e.g. enantiomers, epimers and diastereoisomers), either as individual optical isomers, or mixtures (e.g. racemic mixtures) or two or more optical isomers, unless the context requires otherwise.

For example, the group R¹⁰ can include one or more chiral centres. Thus, for example, when a hydrocarbyl group has two substituents, the carbon atom to which they are attached is typically chiral and hence the compound of the formula (I) will exist as a pair of enantiomers (or more than one pair of enantiomers where more than one chiral centre is present in the compound).

The optical isomers may be characterised and identified by their optical activity (i.e. as + and − isomers, or d and l isomers) or they may be characterised in terms of their absolute stereochemistry using the “R and S” nomenclature developed by Cahn, Ingold and Prelog, see Advanced Organic Chemistry by Jerry March, 4^{th} Edition, John Wiley & Sons, New York, 1992, pages 109-114, and see also Cahn, Ingold & Prelog, Angew. Chem. Int. Ed. Engl., 1966, 5, 385-415.

Optical isomers can be separated by a number of techniques including chiral chromatography (chromatography on a chiral support) and such techniques are well known to the person skilled in the art.

Where compounds of the formula (I) exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention provides compositions containing a compound of the formula (I) having one or more chiral centres, wherein at least 55% (e.g. at least 60%, 65%, 70%, 75%, 80%, 85%, 90% or 95%) of the compound of the formula (I) is present as a single optical isomer (e.g. enantiomer or diastereoisomer). In one general embodiment, 99% or more (e.g. substantially all) of the total amount of the compound of the formula (I) may be present as a single optical isomer (e.g. enantiomer or diastereoisomer).

The compounds of the invention include compounds with one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O.

The isotopes may be radioactive or non-radioactive. In one embodiment of the invention, the compounds contain no radioactive isotopes. Such compounds are preferred for therapeutic use. In another embodiment, however, the compound may contain one or more radioisotopes. Compounds containing such radioisotopes may be useful in a diagnostic context.

Esters such as carboxylic acid esters and acyloxy esters of the compounds of formula (I) bearing a carboxylic acid group or a hydroxyl group are also embraced by Formula (I). Examples of esters are compounds containing the group —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, —C(═O)OCH₃, —C(═O)OCH₂CH₃, —C(═O)OC(CH₃)₃, and —C(═O)OPh. Examples of acyloxy (reverse ester) groups are represented by —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, —OC(═O)CH₃ (acetoxy), —OC(═O)CH₂CH₃, —OC(═O)C(CH₃)₃, —OC(—O)Ph, and —OC(═O)CH₂Ph.

Also encompassed by formula (I) are any polymorphic forms of the compounds, solvates (e.g. hydrates), complexes (e.g. inclusion complexes or clathrates with compounds such as cyclodextrins, or complexes with metals) of the compounds, and pro-drugs of the compounds. By “prodrugs” is meant for example any compound that is converted in vivo into a biologically active compound of the formula (I).

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is:

C₁₋₇alkyl
(e.g., -Me, -Et, -nPr, -iPr, -nBu, -sBu, -iBu, -tBu);
C₁₋₇-aminoalkyl
(e.g., aminoethyl; 2-(N,N-diethylamino)ethyl; 2-(4-morpholino)ethyl); and
acyloxy-C₁₋₇alkyl
(e.g., acyloxymethyl;
acyloxyethyl;
pivaloyloxymethyl;
acetoxymethyl;
1-acetoxyethyl;
1-(1-methoxy-1-methyl)ethyl-carbonxyloxyethyl;
1-(benzoyloxy)ethyl; isopropoxy-carbonyloxymethyl;
1-isopropoxy-carbonyloxyethyl; cyclohexyl-carbonyloxymethyl;
1-cyclohexyl-carbonyloxyethyl;
cyclohexyloxy-carbonyloxymethyl;
1-cyclohexyloxy-carbonyloxyethyl;
(4-tetrahydropyranyloxy)carbonyloxymethyl;
1-(4-tetrahydropyranyloxy)carbonyloxyethyl;
(4-tetrahydropyranyl)carbonyloxymethyl; and
1-(4-tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

### Biological Activity

The compounds of the formulae (I) and sub-groups thereof are inhibitors of cyclin dependent kinases. For example, compounds of the invention are inhibitors of cyclin dependent kinases, and in particular cyclin dependent kinases selected from CDK1, CDK2, CDK3, CDK4, CDK5, CDK6 and CDK9, and more particularly selected from CDK1, CDK2, CDK3, CDK4, CDK5 and CDK9.

Preferred compounds are compounds that inhibit one or more CDK kinases selected from CDK1, CDK2, CDK4 and CDK9, for example CDK1 and/or CDK2 and/or CDK4.

Compounds of the invention also have activity against glycogen synthase kinase-3 (GSK-3).

As a consequence of their activity in modulating or inhibiting CDK and glycogen synthase kinase, they are expected to be useful in providing a means of arresting, or recovering control of, the cell cycle in abnormally dividing cells. It is therefore anticipated that the compounds will prove useful in treating or preventing proliferative disorders such as cancers. It is also envisaged that the compounds of the invention will be useful in treating conditions such as viral infections, type II or non-insulin dependent diabetes mellitus, autoimmune diseases, head trauma, stroke, epilepsy, neurodegenerative diseases such as Alzheimer's, motor neurone disease, progressive supranuclear palsy, corticobasal degeneration and Pick's disease for example autoimmune diseases and neurodegenerative diseases.

One sub-group of disease states and conditions where it is envisaged that the compounds of the invention will be useful consists of viral infections, autoimmune diseases and neurodegenerative diseases.

CDKs play a role in the regulation of the cell cycle, apoptosis, transcription, differentiation and CNS function. Therefore, CDK inhibitors could be useful in the treatment of diseases in which there is a disorder of proliferation, apoptosis or differentiation such as cancer. In particular RB+ve tumours may be particularly sensitive to CDK inhibitors. RB−ve tumours may also be sensitive to CDK inhibitors.

Examples of cancers which may be inhibited include, but are not limited to, a carcinoma, for example a carcinoma of the bladder, breast, colon (e.g. colorectal carcinomas such as colon adenocarcinoma and colon adenoma), kidney, epidermis, liver, lung, for example adenocarcinoma, small cell lung cancer and non-small cell lung carcinomas, oesophagus, gall bladder, ovary, pancreas e.g. exocrine pancreatic carcinoma, stomach, cervix, thyroid, prostate, or skin, for example squamous cell carcinoma; a hematopoietic tumour of lymphoid lineage, for example leukemia, acute lymphocytic leukemia, chronic lymphocytic leukaemia, B-cell lymphoma (such as diffuse large B cell lymphoma), T-cell lymphoma, Hodgkin's lymphoma, non-Hodgkin's lymphoma, hairy cell lymphoma, or Burkett's lymphoma; a hematopoietic tumour of myeloid lineage, for example acute and chronic myelogenous leukemias, myelodysplastic syndrome, or promyelocytic leukemia; thyroid follicular cancer; a tumour of mesenchymal origin, for example fibrosarcoma or rhabdomyosarcoma; a tumour of the central or peripheral nervous system, for example astrocytoma, neuroblastoma, glioma or schwannoma; melanoma; seminoma; teratocarcinoma; osteosarcoma; xeroderma pigmentosum; keratoctanthoma; thyroid follicular cancer; or Kaposi's sarcoma.

The cancers may be cancers which are sensitive to inhibition of any one or more cyclin dependent kinases selected from CDK1, CDK2, CDK3, CDK4, CDK5 and CDK6, for example, one or more CDK kinases selected from CDK1, CDK2, CDK4 and CDK5, e.g. CDK1 and/or CDK2 and/or CDK4.

Whether or not a particular cancer is one which is sensitive to inhibition by a cyclin dependent kinase may be determined by means of a cell growth assay as set out in the examples below or by a method as set out in the section headed “Methods of Diagnosis”.

CDKs are also known to play a role in apoptosis, proliferation, differentiation and transcription and therefore CDK inhibitors could also be useful in the treatment of the following diseases other than cancer; viral infections, for example herpes virus, pox virus, Epstein-Barr virus, Sindbis virus, adenovirus, HIV, HPV, HCV and HCMV; prevention of AIDS development in HIV-infected individuals; chronic inflammatory diseases, for example systemic lupus erythematosus, autoimmune mediated glomerulonephritis, rheumatoid arthritis, psoriasis, inflammatory bowel disease, and autoimmune diabetes mellitus; cardiovascular diseases for example cardiac hypertrophy, restenosis, atherosclerosis; neurodegenerative disorders, for example Alzheimer's disease, AIDS-related dementia, Parkinson's disease, amyotropic lateral sclerosis, retinitis pigmentosa, spinal muscular atropy and cerebellar degeneration; glomerulonephritis; myelodysplastic syndromes, ischemic injury associated myocardial infarctions, stroke and reperfusion injury, arrhythmia, atherosclerosis, toxin-induced or alcohol related liver diseases, haematological diseases, for example, chronic anemia and aplastic anemia; degenerative diseases of the musculoskeletal system, for example, osteoporosis and arthritis, aspirin-sensitive rhinosinusitis, cystic fibrosis, multiple sclerosis, kidney diseases and cancer pain.

It has also been discovered that some cyclin-dependent kinase inhibitors can be used in combination with other anticancer agents. For example, the cyclin-dependent kinase inhibitor flavopiridol has been used with other anticancer agents in combination therapy.

Thus, in the pharmaceutical compositions, uses or methods of this invention for treating a disease or condition comprising abnormal cell growth, the disease or condition comprising abnormal cell growth in one embodiment is a cancer.

One group of cancers includes human breast cancers (e.g. primary breast tumours, node-negative breast cancer, invasive duct adenocarcinomas of the breast, non-endometrioid breast cancers); and mantle cell lymphomas. In addition, other cancers are colorectal and endometrial cancers.

Another sub-set of cancers includes hematopoietic tumours of lymphoid lineage, for example leukemia, chronic lymphocytic leukaemia, mantle cell lymphoma and B-cell lymphoma (such as diffuse large B cell lymphoma).

One particular cancer is chronic lymphocytic leukaemia.

Another particular cancer is mantle cell lymphoma.

Another particular cancer is diffuse large B cell lymphoma

Another sub-set of cancers includes breast cancer, ovarian cancer, colon cancer, prostate cancer, oesophageal cancer, squamous cancer and non-small cell lung carcinomas.

A further sub-set of cancers, namely cancers wherein compounds having CDK4 inhibitory activity may be of particular therapeutic benefit, comprises retinoblastomas, small cell lung carcinomas, non-small lung carcinomas, sarcomas, gliomas, pancreatic cancers, head, neck and breast cancers and mantle cell lymphomas.

Another sub-set of cancers wherein compounds having CDK4 inhibitory activity may be of particular therapeutic benefit comprises small cell lung cancer, non-small cell lung cancer, pancreatic cancer, breast cancer, glioblastoma multiforme, T cell ALL and mantle cell lymphoma.

The activity of the compounds of the invention as inhibitors of cyclin dependent kinases and glycogen synthase kinase-3 can be measured using the assays set forth in the examples below and the level of activity exhibited by a given compound can be defined in terms of the IC₅₀ value. Preferred compounds of the present invention are compounds having an IC₅₀ value of less than 1 micromolar, more preferably less than 0.1 micromolar.

### Methods for the Preparation of Compounds of the Invention

Compounds of the formula (I) and the various sub-groups thereof can be prepared in accordance with synthetic methods well known to the skilled person and the methods described in the Examples section of this application. Unless stated otherwise, R¹, R², R³, Y, X and A are as hereinbefore defined.

In this section, as in all the other sections of this application, references to formula (I) should be taken to refer also to formulae (Ia), (Ib), (Ic), (Id), (Ie), (II), (IIa), (IIb), (III), (IIIa), (IIIb), (IV), (V), (Va), (Vb), (VI), (VIa) or (VII) and sub-groups thereof unless the context indicates otherwise.

Compounds of the formula (I) can be prepared by the reaction of a carboxylic acid of the formula (X), R^{h}=H, or an activated derivative thereof with an amine of the formula H₂N—Y—R³ under conditions suitable for forming an amide bond.

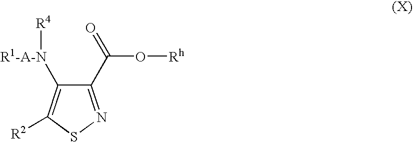

The coupling reaction between the carboxylic acid (X) and the amine is preferably carried out in the presence of a reagent of the type commonly used in the formation of peptide linkages. Examples of such reagents include 1,3-dicyclohexylcarbodiimide (DCC) (Sheehan et al, J. Amer. Chem. Soc. 1955, 77, 1067), 1-ethyl-3-(3′-dimethylaminopropyl)-carbodiimide (referred to herein either as EDC or EDAC but also known in the art as EDCI and WSCDI) (Sheehan et al, J. Org. Chem., 1961, 26, 2525), uronium-based coupling agents such as O-(7-azabenzotriazol-1-yl)-N,N,N′,N′-tetramethyluronium hexafluorophosphate (HATU) and phosphonium-based coupling agents such as 1-benzo-triazolyloxytrispyrrolidino)phosphonium hexafluorophosphate (PyBOP) (Castro et al, Tetrahedron Letters, 1990, 31, 205). Carbodiimide-based coupling agents are advantageously used in combination with 1-hydroxy-7-azabenzotriazole (HOAt) (L. A. Carpino, J. Amer. Chem. Soc., 1993, 115, 4397) or 1-hydroxybenzotriazole (HOBt) (Konig et al, Chem. Ber., 103, 708, 2024-2034). Preferred coupling reagents include EDC (EDAC) and DCC in combination with HOAt or HOBt.

The coupling reaction is typically carried out in a non-aqueous, non-protic solvent such as acetonitrile, dioxan, dimethylsulphoxide, dichloromethane, dimethylformamide or N-methylpyrrolidine, or in an aqueous solvent optionally together with one or more miscible co-solvents. The reaction can be carried out at room temperature or, where the reactants are less reactive (for example in the case of electron-poor anilines bearing electron withdrawing groups such as sulphonamide groups) at an appropriately elevated temperature. The reaction may be carried out in the presence of a non-interfering base, for example a tertiary amine such as triethylamine or N,N-diisopropylethylamine.

As an alternative, a reactive derivative of the carboxylic acid, e.g. an anhydride or acid chloride, may be used. Reaction with a reactive derivative such an anhydride is typically accomplished by stirring the amine and anhydride at room temperature in the presence of a base such as pyridine. Reaction with an acid chloride is typically carried out in an aprotic solvent such as dichloromethane in the presence of a non-interfering base such as triethylamine.

Amines of the formula H₂N—Y—R³ can be obtained from commercial sources or can be prepared by any of a large number of standard synthetic methods well known those skilled in the art, see for example see Advanced Organic Chemistry by Jerry March, 4^{th} Edition, John Wiley & Sons, 1992, and Organic Syntheses, Volumes 1-8, John Wiley, edited by Jeremiah P. Freeman (ISBN: 0-471-31192-8), 1995.

The carboxylic acids of the formula (X), R^{h}=H, can be prepared by hydrolysis of the corresponding ester (e.g. ethyl ester) under standard conditions for the saponification of esters. Thus, for example, the esters can be treated with an alkali metal hydroxide such as sodium hydroxide in a polar solvent such as dioxan, for example at room temperature or with mild heating.

Esters of the formula (X), R^{h}=ethyl) wherein A is C═O can be prepared from 4-aminoisothiazole compounds of the formula (X¹):

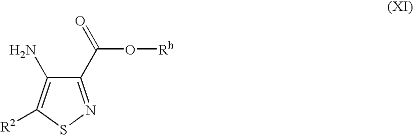

by reaction with a carboxylic acid of the formula R¹CO₂H Under Amide Coupling conditions as described above.

Where A is NH(C═O), the compound of the formula (X) can be prepared by reacting an amino-isothiazole compound of the formula (XI) with a suitably substituted phenylisocyanate in a polar solvent such as DMF. The reaction is conveniently carried out at room temperature. Ureas may alternatively be formed by reacting the ester (XI) with an amine R¹—NH₂ in the presence of a “carbonyl donating” reagent such as carbonyl dimidazole (CDI) or triphosgene.

Alternatively, where A is O(C═O) the compound of the formula (X) can be prepared by reacting an amino-isothiazole compound of the formula (X¹) with a chloroformate derivative of the formula R¹—O—C(O)—Cl under conditions well known to the skilled person.

In an alternative route to compounds of the formula (I), a 4-aminoisothiazole compound of the formula (XII) can be reacted with a carboxylic acid of the formula R¹CO₂H or an activated derivative thereof under amide coupling conditions of the type described above.

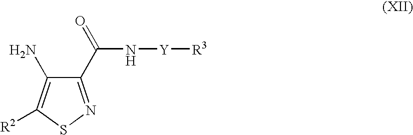

Amine compounds of the formula (XII) can be prepared from the corresponding 4-nitro compound by reaction with a suitable reducing agent. For example, reduction may be effected using a reducing agent such as iron or tin (II) chloride in ethanol, typically with heating, for example to the reflux temperature of the solvent.

Carboxylic acids of the formula R¹—CO₂H can be obtained commercially or can be synthesised according to methods well known to the skilled person, see for example Advanced Organic Chemistry and Organic Syntheses, the details for which are given above.

Compounds of the formula (I) in which A is a bond, can be prepared from the 4-amino compounds of the formula (XII) by a number of methods. Reductive amination with an appropriately substituted aldehyde or ketone can be carried out in the presence of variety of reducing agents (see Advanced Organic Chemistry by Jerry March, 4^{th} Edition, John Wiley & Sons, 1992, pp 898-900. For example, reductive amination can be carried out in the presence of sodium triacetoxyborohydride in the presence of an aprotic solvent such as dichloromethane at or near ambient temperatures.

Compounds in which X is a group R¹-A-NR⁴ where A is a bond can also be prepared by the reaction of the 4-amino isothiazole compound (XII) with a compound of the formula R¹-L in a nucleophilic displacement reaction where L is a leaving group such as a halogen.

Compounds of the formula (XII) can also be converted into urea or urethane compounds of the formula (I) by means of the methods described above.

Thiazole compounds of the invention (i.e. wherein, in formula (I), J is CH and Q is S) can be prepared by the sequence of reactions shown in Scheme 1.

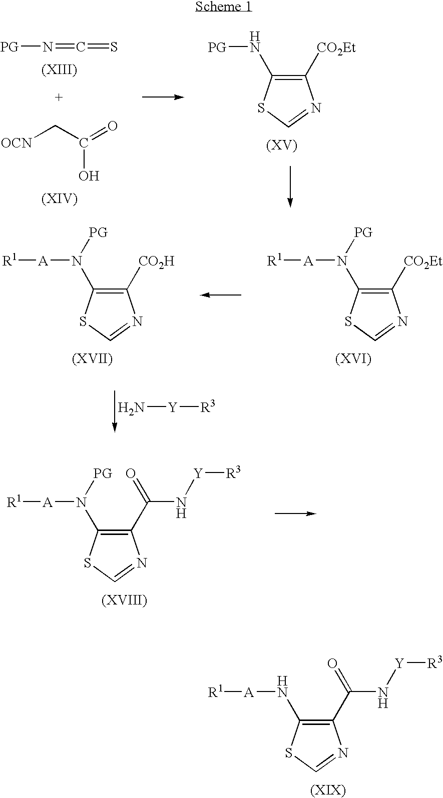

In Scheme 1, ethyl isocyanoacetate (XIV) is reacted with a substituted isothiocyanate (XIII) in which PG is a protecting group such as p-methoxybenzyl to form thiazole ester (XV). The reaction is typically carried out in a polar solvent such as THF in the presence of a strong base such as potassium tert-butoxide, for example at room temperature.

The thiazole ester is then converted into the ester compound (XVI) by removing the protecting group and reacting the resulting amine with a carboxylic acid or active derivative thereof under amide forming conditions of the type described above, or by reaction with appropriately substituted isocyanate or amine under urea forming conditions, also as described. Alternatively, when the protecting group is a benzylic group such as para-methoxybenzyl, the compound of formula (XV) may be treated with a strong base, e.g. a metal hydride such as sodium hydride, and then reacted with an acid chloride of the formula R¹—COCl to form the intermediate (XVI). The reaction with the acid chloride is typically carried out at room temperature in a polar aprotic solvent such as DMF.

The ester compound (XVI) is then hydrolysed using an alkali metal hydroxide such as sodium hydroxide to give the carboxylic acid (XVII).

The carboxylic acid (XVII) can then be converted into amide (XVIII) by reaction with an amine H₂N—Y—R³ under the amide conditions described above, for example using a combination of EDC and HOBt as the amide coupling reagent in dichloromethane solvent at room temperature.

As the final step, the protecting group PG and any other protecting groups present can be removed by standard methods to give the amide (XIX). For example, when PG is apara-methoxybenzyl group, this can be removed using trifluoroacetic acid in the presence of anisole.

In Scheme 1, the N-protected carboxylic acid (XVII) is reacted with the amine H₂N—Y—R³ to give the protected amide compound of formula (XVIII) which is then deprotected to give the product (XIX). In an alternative to this route, the carboxylic acid (XXI):

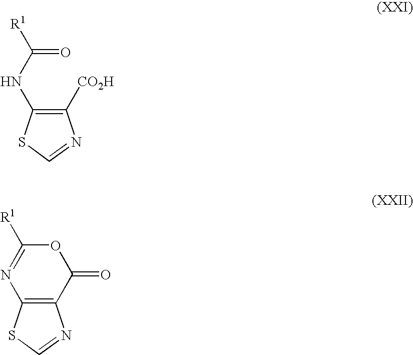

can be converted to the imidate lactone (XXII) by reaction with oxalyl chloride in a an aprotic solvent such as dichloromethane in the presence of dimethylformamide. The imidate lactone (XXII) can then be reacted with the amine H₂N—Y—R³ at an elevated temperature (i.e. above 100° C., more usually at least 130° C., for example approximately 150° C. to give a compound of the formula (I). The reaction is typically carried out in a high boiling polar solvent such as NMP using microwave heating.

In order to prepare thiazole compounds of the formula (I) wherein A is a bond and R¹ is, for example, a heteroaryl, aryl, heteroarylalkyl or arylalkyl group, a compound of the formula R¹—N═C═S can be reacted with the isocyanoacetate (XIV) to give a compound of the formula ((XX):

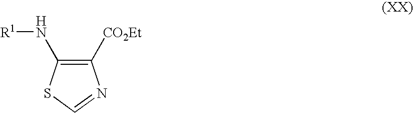

which can then be hydrolysed using sodium hydroxide and coupled with an amine H₂N—Y—R³ under the amide conditions described above.

Once formed, one compound of the formula (I) may be transformed into another compound of the formula (I) using standard chemistry procedures well known in the art. For examples of functional group interconversions, see for example, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8), 1995.

### Protecting Groups

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999).

A hydroxy group may be protected, for example, as an ether (—OR) or an ester (—OC(═O)R), for example, as: a t-butyl ether; a tetrahydropyranyl (THP) ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc).

An aldehyde or ketone group may be protected, for example, as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

An amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz or NH-Z); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), or as a 2(-phenylsulphonyl)ethyloxy amide (—NH-Psec).

Other protecting groups for amines, such as cyclic amines and heterocyclic N—H groups, include toluenesulphonyl (tosyl) and methanesulphonyl (mesyl) groups, benzyl groups such as apara-methoxybenzyl (PMB) group and tetrahydropyranyl (THP) groups.

A carboxylic acid group may be protected as an ester for example, as: an C₁₋₇ alkyl ester (e.g., a methyl ester; a t-butyl ester); a C₁₋₇ haloalkyl ester (e.g., a C₁₋₇ trihaloalkyl ester); a triC₁₋₇ alkylsilyl-C₁₋₇alkyl ester; or a C₅₋₂₀ aryl-C₁₋₇ alkyl ester (e.g., a benzyl ester; a nitrobenzyl ester); or as an amide, for example, as a methyl amide. A thiol group may be protected, for example, as a thioether (—SR), for example, as: a benzyl thioether; an acetamidomethyl ether (—S—CH₂NHC(—O)CH₃).

### Isolation and Purification of the Compounds of the Invention

The compounds of the invention can be isolated and purified according to standard techniques well known to the person skilled in the art. One technique of particular usefulness in purifying the compounds is preparative liquid chromatography using mass spectrometry as a means of detecting the purified compounds emerging from the chromatography column.

Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z, Lindsley C., Development of a custom high-throughput preparative liquid chromatography/mass spectrometer platform for the preparative purification and analytical analysis of compound libraries; J Comb Chem.; 2003; 5(3); 322-9.

An example of such a system for purifying compounds via preparative LC-MS is described below in the Examples section of this application (under the heading “Mass Directed Purification LC-MS System”). However, it will be appreciated that alternative systems and methods to those described could be used. In particular, normal phase preparative LC based methods might be used in place of the reverse phase methods described here. Most preparative LC-MS systems utilise reverse phase LC and volatile acidic modifiers, since the approach is very effective for the purification of small molecules and because the eluents are compatible with positive ion electrospray mass spectrometry. Employing other chromatographic solutions e.g. normal phase LC, alternatively buffered mobile phase, basic modifiers etc as outlined in the analytical methods described below could alternatively be used to purify the compounds.

### Pharmaceutical Formulations

While it is possible for the active compound to be administered alone, it is preferable to present it as a pharmaceutical composition (e.g. formulation) comprising at least one active compound of the invention together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents; for example agents that reduce or alleviate some of the side effects associated with chemotherapy. Particular examples of such agents include anti-emetic agents and agents that prevent or decrease the duration of chemotherapy-associated neutropenia and prevent complications that arise from reduced levels of red blood cells or white blood cells, for example erythropoietin (EPO), granulocyte macrophage-colony stimulating factor (GM-CSF), and granulocyte-colony stimulating factor (G-CSF).

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Accordingly, in a further aspect, the invention provides compounds of the formula (I) and sub-groups thereof as defined herein in the form of pharmaceutical compositions.

The pharmaceutical compositions can be in any form suitable for oral, parenteral, topical, intranasal, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer term infusion and can be via passive delivery or through the utilisation of a suitable infusion pump.

Pharmaceutical formulations adapted for parenteral administration include aqueous and non-aqueous sterile injection solutions which may contain anti-oxidants, buffers, bacteriostats, co-solvents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, inter alia, stabilising the active ingredient in a soluble form and rendering the formulation isotonic with the blood of the intended recipient. Pharmaceutical formulations for parenteral administration may also take the form of aqueous and non-aqueous sterile suspensions which may include suspending agents and thickening agents (R. G. Strickly, Solubilizing Excipients in oral and injectable formulations, Pharmaceutical Research, Vol 21(2) 2004, p 201-230).

A drug molecule that is ionizable can be solubilized to the desired concentration by pH adjustment if the drug's pKₐ is sufficiently away from the formulation pH value. The acceptable range is pH 2-12 for intravenous and intramuscular administration, but subcutaneously the range is pH 2.7-9.0. The solution pH is controlled by either the salt form of the drug, strong acids/bases such as hydrochloric acid or sodium hydroxide, or by solutions of buffers which include but are not limited to buffering solutions formed from glycine, citrate, acetate, maleate, succinate, histidine, phosphate, tris(hydroxymethyl)aminomethane (TRIS), or carbonate.

The combination of an aqueous solution and a water-soluble organic solvent/surfactant (i.e., a cosolvent) is often used in injectable formulations. The water-soluble organic solvents and surfactants used in injectable formulations include but are not limited to propylene glycol, ethanol, polyethylene glycol 300, polyethylene glycol 400, glycerin, dimethylacetamide (DMA), N-methyl-2-pyrrolidone (NIP; Pharmasolve), dimethylsulphoxide (DMSO), Solutol HS 15, Cremophor EL, Cremophor RH 60, and polysorbate 80. Such formulations can usually be, but are not always, diluted prior to injection.

Propylene glycol, PEG 300, ethanol, Cremophor EL, Cremophor RH 60, and polysorbate 80 are the entirely organic water-miscible solvents and surfactants used in commercially available injectable formulations and can be used in combinations with each other. The resulting organic formulations are usually diluted at least 2-fold prior to IV bolus or IV infusion.

Alternatively increased water solubility can be achieved through molecular complexation with cyclodextrins

Liposomes are closed spherical vesicles composed of outer lipid bilayer membranes and an inner aqueous core and with an overall diameter of <100 μm. Depending on the level of hydrophobicity, moderately hydrophobic drugs can be solubilized by liposomes if the drug becomes encapsulated or intercalated within the liposome. Hydrophobic drugs can also be solubilized by liposomes if the drug molecule becomes an integral part of the lipid bilayer membrane, and in this case, the hydrophobic drug is dissolved in the lipid portion of the lipid bilayer. A typical liposome formulation contains water with phospholipid at −5-20 mg/ml, an isotonicifier, a pH 5-8 buffer, and optionally cholesterol.

The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use.

The pharmaceutical formulation can be prepared by lyophilising a compound of Formula (I) or acid addition salt thereof. Lyophilisation refers to the procedure of freeze-drying a composition. Freeze-drying and lyophilisation are therefore used herein as synonyms. A typical process is to solubilise the compound and the resulting formulation is clarified, sterile filtered and aseptically transferred to containers appropriate for lyophilisation (e.g. vials). In the case of vials, they are partially stoppered with lyo-stoppers. The formulation can be cooled to freezing and subjected to lyophilisation under standard conditions and then hermetically capped forming a stable, dry lyophile formulation. The composition will typically have a low residual water content, e.g. less than 5% e.g. less than 1% by weight based on weight of the lyophile.

The lyophilisation formulation may contain other excipients for example, thickening agents, dispersing agents, buffers, antioxidants, preservatives, and tonicity adjusters. Typical buffers include phosphate, acetate, citrate and glycine. Examples of antioxidants include ascorbic acid, sodium bisulphite, sodium metabisulphite, monothioglycerol, thiourea, butylated hydroxytoluene, butylated hydroxyl anisole, and ethylenediamietetraacetic acid salts. Preservatives may include benzoic acid and its salts, sorbic acid and its salts, alkyl esters of para-hydroxybenzoic acid, phenol, chlorobutanol, benzyl alcohol, thimerosal, benzalkonium chloride and cetylpyridinium chloride. The buffers mentioned previously, as well as dextrose and sodium chloride, can be used for tonicity adjustment if necessary.

Bulking agents are generally used in lyophilisation technology for facilitating the process and/or providing bulk and/or mechanical integrity to the lyophilized cake.

Bulking agent means a freely water soluble, solid particulate diluent that when co-lyophilised with the compound or salt thereof, provides a physically stable lyophilized cake, a more optimal freeze-drying process and rapid and complete reconstitution. The bulking agent may also be utilised to make the solution isotonic.

The water-soluble bulking agent can be any of the pharmaceutically acceptable inert solid materials typically used for lyophilisation. Such bulking agents include, for example, sugars such as glucose, maltose, sucrose, and lactose; polyalcohols such as sorbitol or mannitol; amino acids such as glycine; polymers such as polyvinylpyrrolidine; and polysaccharides such as dextran.

The ratio of the weight of the bulking agent to the weight of active compound is typically within the range from about 1 to about 5, for example of about 1 to about 3, e.g. in the range of about 1 to 2.

Alternatively they can be provided in a solution form which may be concentrated and sealed in a suitable vial. Sterilisation of dosage forms may be via filtration or by autoclaving of the vials and their contents at appropriate stages of the formulation process. The supplied formulation may require further dilution or preparation before delivery for example dilution into suitable sterile infusion packs.

Extemporaneous injection solutions and suspensions may be prepared from sterile powders, granules and tablets.

In one preferred embodiment of the invention, the pharmaceutical composition is in a form suitable for i.v. administration, for example by injection or infusion.

In another preferred embodiment, the pharmaceutical composition is in a form suitable for sub-cutaneous (s.c.) administration.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Pharmaceutical compositions containing compounds of the formula (I) can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (eg; tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. As a further alternative, the active compound can be formulated in a delivery system that provides osmotic control of the release of the compound. Osmotic release and other delayed release or sustained release formulations may be prepared in accordance with methods well known to those skilled in the art.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

Compositions for topical use include ointments, creams, sprays, patches, gels, liquid drops and inserts (for example intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the formula (I) will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within this range, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 milligrams to 1 gram, of active compound.

The active compound will be administered to a patient in need thereof (for example a human or animal patient) in an amount sufficient to achieve the desired therapeutic effect.

### Methods of Treatment

It is envisaged that the compounds of the formula (I) and sub-groups thereof as defined herein will be useful in the prophylaxis or treatment of a range of disease states or conditions mediated by cyclin dependent kinases and glycogen synthase kinase-3. Examples of such disease states and conditions are set out above.

The compounds are generally administered to a subject in need of such administration, for example a human or animal patient, preferably a human.

The compounds will typically be administered in amounts that are therapeutically or prophylactically useful and which generally are non-toxic. However, in certain situations (for example in the case of life threatening diseases), the benefits of administering a compound of the formula (I) may outweigh the disadvantages of any toxic effects or side effects, in which case it may be considered desirable to administer compounds in amounts that are associated with a degree of toxicity.

The compounds may be administered over a prolonged term to maintain beneficial therapeutic effects or may be administered for a short period only. Alternatively they may be administered in a pulsatile or continuous manner.

A typical daily dose of the compound of formula (I) can be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams, and more typically 1 microgram per kilogram to 20 milligrams per kilogram, for example 1 microgram to 10 milligrams per kilogram) per kilogram of bodyweight although higher or lower doses may be administered where required. The compound of the formula (I) can be administered on a daily basis or on a repeat basis every 2, or 3, or 4, or 5, or 6, or 7, or 10 or 14, or 21, or 28 days for example.

An example of a dosage for a 60 kilogram person comprises administering a compound of the formula (I) as defined herein at a starting dosage of 4.5-10.8 mg/60 kg/day (equivalent to 75-180 μg/kg/day) and subsequently by an efficacious dose of 44-97 mg/60 kg/day (equivalent to 0.7-1.6 mg/kg/day) or an efficacious dose of 72-274 mg/60 kg/day (equivalent to 1.2-4.6 mg/kg/day) although higher or lower doses may be administered where required. The mg/kg dose would scale pro-rata for any given body weight.

In one particular dosing schedule, a patient will be given an infusion of a compound of the formula (I) for periods of one hour daily for up to ten days in particular up to five days for one week, and the treatment repeated at a desired interval such as two to four weeks, in particular every three weeks.

More particularly, a patient may be given an infusion of a compound of the formula (I) for periods of one hour daily for 5 days and the treatment repeated every three weeks.

In another particular dosing schedule, a patient is given an infusion over 30 minutes to 1 hour followed by maintenance infusions of variable duration, for example 1 to 5 hours, e.g. 3 hours.

In a further particular dosing schedule, a patient is given a continuous infusion for a period of 12 hours to 5 days, an in particular a continuous infusion of 24 hours to 72 hours.

Ultimately, however, the quantity of compound administered and the type of composition used will be commensurate with the nature of the disease or physiological condition being treated and will be at the discretion of the physician.

The compounds of formula (I) and sub-groups as defined herein can be administered as the sole therapeutic agent or they can be administered in combination therapy with one of more other compounds for treatment of a particular disease state, for example a neoplastic disease such as a cancer as hereinbefore defined. Examples of other therapeutic agents or therapies that may be administered or used together (whether concurrently or at different time intervals) with the compounds of the invention include but are not limited to topoisomerase inhibitors, alkylating agents, antimetabolites, DNA binders, microtubule inhibitors (tubulin targeting agents), monoclonal antibodies and signal transduction inhibitors, particular examples being cisplatin, cyclophosphamide, doxorubicin, irinotecan, fludarabine, 5FU, taxanes, mitomycin C and radiotherapy.

For the case of CDK inhibitors combined with other therapies, the two or more treatments may be given in individually varying dose schedules and via different routes.

Where the compound of the formula (I) is administered in combination therapy with one, two, three, four or more other therapeutic agents (preferably one or two, more preferably one), the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

The compounds of the invention may also be administered in conjunction with non-chemotherapeutic treatments such as radiotherapy, photodynamic therapy, gene therapy; surgery and controlled diets.

For use in combination therapy with another chemotherapeutic agent, the compound of the formula (I) and one, two, three, four or more other therapeutic agents can be, for example, formulated together in a dosage form containing two, three, four or more therapeutic agents. In an alternative, the individual therapeutic agents may be formulated separately and presented together in the form of a kit, optionally with instructions for their use.

A person skilled in the art would know through his or her common general knowledge the dosing regimes and combination therapies to use.

### Methods of Diagnosis

Prior to administration of a compound of the formula (I), a patient may be screened to determine whether a disease or condition from which the patient is or may be suffering is one which would be susceptible to treatment with a compound having activity against cyclin dependent kinases.

For example, a biological sample taken from a patient may be analysed to determine whether a condition or disease, such as cancer, that the patient is or may be suffering from is one which is characterised by a genetic abnormality or abnormal protein expression which leads to over-activation of CDKs or to sensitisation of a pathway to normal CDK activity. Examples of such abnormalities that result in activation or sensitisation of the CDK2 signal include up-regulation of cyclin E, (Harwell R M, Mull B B, Porter D C, Keyomarsi K.; J Biol. Chem. 2004 Mar. 26; 279(13):12695-705) or loss of p21 or p27, or presence of CDC4 variants (Rajagopalan H, Jallepalli P V, Rago C, Velculescu V E, Kinzler K W, Vogelstein B, Lengauer C.; Nature. 2004 Mar. 4; 428(6978):77-81). Tumours with mutants of CDC4 or up-regulation, in particular over-expression, of cyclin E or loss of p21 or p27 may be particularly sensitive to CDK inhibitors. The term up-regulation includes elevated expression or over-expression, including gene amplification (i.e. multiple gene copies) and increased expression by a transcriptional effect, and hyperactivity and activation, including activation by mutations.

Thus, the patient may be subjected to a diagnostic test to detect a marker characteristic of up-regulation of cyclin E, or loss of p21 or p27, or presence of CDC4 variants. The term diagnosis includes screening. By marker we include genetic markers including, for example, the measurement of DNA composition to identify mutations of CDC4. The term marker also includes markers which are characteristic of up regulation of cyclin E, including enzyme activity, enzyme levels, enzyme state (e.g. phosphorylated or not) and mRNA levels of the aforementioned proteins. Tumours with upregulation of cyclin E, or loss of p21 or p27 may be particularly sensitive to CDK inhibitors. Tumours may preferentially be screened for upregulation of cyclin E, or loss of p21 or p27 prior to treatment. Thus, the patient may be subjected to a diagnostic test to detect a marker characteristic of up-regulation of cyclin E, or loss of p21 or p27.

In addition, the cancer may be analysed for INK4a and RB loss of function, and cyclin D1 or CDK4 overexpression or CDK4 mutation. RB loss and mutations inactivating p16^{INK4a} function or hypermethylation of p16^{INK4a} occurs in many tumor types. Rb is inactivated in 100% retinoblastomas and in 90% of small cell lung carcinomas. Cyclin D1 is amplified in 40% of head and neck, over-expressed in 50% of breast cancers and 90% of mantle cell lymphomas. p16 deleted in 60% of non-small lung carcinomas and in 40% of pancreatic cancers. CDK4 amplified in 20% of sarcomas and in 10% of gliomas. Events resulting in RB or p16^{INK4a} inactivation through mutation, deletion, or epigenetic silencing, or in the overexpression of cyclin D1 or Cdk4 can be identified by the techniques outlined herein. Tumours with up-regulation, in particular over-expression of cyclin D or CDK4 or loss of INK4a or RB may be particularly sensitive to CDK inhibitors. Thus, the patient may be subjected to a diagnostic test to detect a marker characteristic of over-expression of cyclin D or CDK4 or loss of INK4a or RB.

Cancers that experience INK4a and RB loss of function and cyclin D1 or CDK4 overexpression, include small cell lung cancer, non-small cell lung cancer, pancreatic cancer, breast cancer, glioblastoma multiforme, T cell ALL and mantle cell lymphoma. Therefore patients with small cell lung cancer, non-small cell lung cancer, pancreatic cancer, breast cancer, glioblastoma multiforme, T cell ALL or mantle cell lymphoma could be selected for treatment with a CDK inhibitor using diagnostic tests outlined above and may in particular be treated with a CDK inhibitor as provided herein.

The diagnostic tests are typically conducted on a biological sample selected from tumour biopsy samples, blood samples (isolation and enrichment of shed tumour cells), stool biopsies, sputum, chromosome analysis, pleural fluid, peritoneal fluid, or urine.

It has been found, Rajagopalan et al (Nature. 2004 Mar. 4; 428(6978):77-81), that there were mutations present in CDC4 (also known as Fbw7 or Archipelago) in human colorectal cancers and endometrial cancers (Spruck et al, Cancer Res. 2002 Aug. 15; 62(16):4535-9). Identification of individual carrying a mutation in CDC4 may mean that the patient would be particularly suitable for treatment with a CDK inhibitor. Tumours may preferentially be screened for presence of a CDC4 variant prior to treatment. The screening process will typically involve direct sequencing, oligonucleotide microarray analysis, or a mutant specific antibody.

Methods of identification and analysis of mutations and up-regulation of proteins are well known to a person skilled in the art. Screening methods could include, but are not limited to, standard methods such as reverse-transcriptase polymerase chain reaction (RT-PCR) or in-situ hybridisation.

In screening by RT-PCR, the level of mRNA in the tumour is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. Current Protocols in Molecular Biology, 2004, John Wiley & Sons Inc., or Innis, M. A. et-al., eds. PCR Protocols: a guide to methods and applications, 1990, Academic Press, San Diego. Reactions and manipulations involving nucleic acid techniques are also described in Sambrook et al., 2001, 3^{rd} Ed, Molecular Cloning: A Laboratory Manual, Cold Spring Harbor Laboratory Press. Alternatively a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference.

An example of an in-situ hybridisation technique for assessing mRNA expression would be fluorescence in-situ hybridisation (FISH) (see Angerer, 1987 Meth. Enzymol., 152: 649).

Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. Current Protocols in Molecular Biology, 2004, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Alternatively, the protein products expressed from the mRNAs may be assayed by immunohistochemistry of tumour samples, solid phase immunoassay with microtiter plates, Western blotting, 2-dimensional SDS-polyacrylamide gel electrophoresis, ELISA, flow cytometry and other methods known in the art for detection of specific proteins. Detection methods would include the use of site specific antibodies. The skilled person will recognize that all such well-known techniques for detection of upregulation of cyclin E, or loss of p21 or p27, or detection of CDC4 variants could be applicable in the present case.

Therefore, all of these techniques could also be used to identify tumours particularly suitable for treatment with the compounds of the invention.

Tumours with mutants of CDC4 or up-regulation, in particular over-expression, of cyclin E or loss of p21 or p27 may be particularly sensitive to CDK inhibitors. Tumours may preferentially be screened for up-regulation, in particular over-expression, of cyclin E (Harwell R M, Mull B B, Porter D C, Keyomarsi K.; J Biol. Chem. 2004 Mar. 26; 279(13):12695-705) or loss of p21 or p27 or for CDC4 variants prior to treatment (Rajagopalan H, Jallepalli P V, Rago C, Velculescu V E, Kinzler K W, Vogelstein B, Lengauer C.; Nature. 2004 Mar. 4; 428(6978):77-81).

Patients with mantle cell lymphoma (MCL) could be selected for treatment with a compound of the invention using diagnostic tests outlined herein. MCL is a distinct clinicopathologic entity of non-Hodgkin's lymphoma, characterized by proliferation of small to medium-sized lymphocytes with co-expression of CD5 and CD20, an aggressive and incurable clinical course, and frequent t(11;14)(q13;q32) translocation. Over-expression of cyclin D1 mRNA, found in mantle cell lymphoma (MCL), is a critical diagnostic marker. Yatabe et al (Blood. 2000 Apr. 1; 95(7):2253-61) proposed that cyclin D1-positivity should be included as one of the standard criteria for MCL, and that innovative therapies for this incurable disease should be explored on the basis of the new criteria. Jones et al (J Mol. Diagn. 2004 May; 6(2):84-9) developed a real-time, quantitative, reverse transcription PCR assay for cyclin D1 (CCND1) expression to aid in the diagnosis of mantle cell lymphoma (MCL). Howe et al (Clin Chem. 2004 January; 50(1):80-7) used real-time quantitative RT-PCR to evaluate cyclin D1 mRNA expression and found that quantitative RT-PCR for cyclin D1 mRNA normalized to CD19 mRNA can be used in the diagnosis of MCL in blood, marrow, and tissue. Alternatively, patients with breast cancer could be selected for treatment with a CDK inhibitor using diagnostic tests outline above. Tumour cells commonly overexpress cyclin E and it has been shown that cyclin E is over-expressed in breast cancer (Harwell et al, Cancer Res, 2000, 60, 481-489). Therefore breast cancer may in particular be treated with a CDK inhibitor as provided herein.

### Antifungal Use

In a further aspect, the invention provides the use of the compounds of the formula (I) and sub-groups thereof as defined herein as antifungal agents.

The compounds may be used in animal medicine (for example in the treatment of mammals such as humans), or in the treatment of plants (e.g. in agriculture and horticulture), or as general antifungal agents, for example as preservatives and disinfectants.

In one embodiment, the invention provides compounds of the formula (I) and sub-groups thereof as defined herein for use in the prophylaxis or treatment of a fungal infection in a mammal such as a human.

Also provided is the use of a compound of the formula (I) and sub-groups thereof as defined herein for the manufacture of a medicament for use in the prophylaxis or treatment of a fungal infection in a mammal such as a human.

For example, compounds of the invention may be administered to human patients suffering from, or at risk of infection by, topical fungal infections caused by among other organisms, species of Candida, Trichophyton, Microsporum or Epidermophyton, or in mucosal infections caused by Candida albicans (e.g. thrush and vaginal candidiasis). The compounds of the invention can also be administered for the treatment or prophylaxis of systemic fungal infections caused by, for example, Candida albicans, Cryptococcus neoformans, Aspergillus flavus, Aspergillus fumigatus, Coccidiodies, Paracoccidioides, Histoplasma or Blastomyces.

In another aspect, the invention provides an antifungal composition for agricultural (including horticultural) use, comprising a compound of the formulae (I) and sub-groups thereof as defined herein together with an agriculturally acceptable diluent or carrier.

The invention further provides a method of treating an animal (including a mammal such as a human), plant or seed having a fungal infection, which comprises treating said animal, plant or seed, or the locus of said plant or seed, with an effective amount of a compound of formula and sub-groups thereof as defined herein.

The invention also provides a method of treating a fungal infection in a plant or seed which comprises treating the plant or seed with an antifungally effective amount of a fungicidal composition as hereinbefore defined.

Differential screening assays may be used to select for those compounds of the present invention with specificity for non-human CDK enzymes. Compounds which act specifically on the CDK enzymes of eukaryotic pathogens can be used as anti-fungal or anti-parasitic agents. Inhibitors of the Candida CDK kinase, CKSI, can be used in the treatment of candidiasis. Antifungal agents can be used against infections of the type hereinbefore defined, or opportunistic infections that commonly occur in debilitated and immunosuppressed patients such as patients with leukemias and lymphomas, people who are receiving immunosuppressive therapy, and patients with predisposing conditions such as diabetes mellitus or AIDS, as well as for non-immunosuppressed patients.

Assays described in the art can be used to screen for agents which may be useful for inhibiting at least one fungus implicated in mycosis such as candidiasis, aspergillosis, mucormycosis, blastomycosis, geotrichosis, cryptococcosis, chromoblastomycosis, coccidiodomycosis, conidiosporosis, histoplasmosis, maduromycosis, rhinosporidosis, nocaidiosis, para-actinomycosis, penicilliosis, monoliasis, or sporotrichosis. The differential screening assays can be used to identify anti-fungal agents which may have therapeutic value in the treatment of aspergillosis by making use of the CDK genes cloned from yeast such as Aspergillus fumigatus, Aspergillus flavus, Aspergillus niger, Aspergillus nidulans, or Aspergillus terreus, or where the mycotic infection is mucon-nycosis, the CDK assay can be derived from yeast such as Rhizopus arrhizus, Rhizopus oryzae, Absidia corymbifera, Absidia ramosa, or Mucor pusillus. Sources of other CDK enzymes include the pathogen Pneumocystis carinii.

By way of example, in vitro evaluation of the antifungal activity of the compounds can be performed by determining the minimum inhibitory concentration (M.I.C.) which is the concentration of the test compounds, in a suitable medium, at which growth of the particular microorganism fails to occur. In practice, a series of agar plates, each having the test compound incorporated at a particular concentration is inoculated with a standard culture of, for example, Candida albicans and each plate is then incubated for an appropriate period at 37° C. The plates are then examined for the presence or absence of growth of the fungus and the appropriate M.I.C. value is noted

The in vivo evaluation of the compounds can be carried out at a series of dose levels by intraperitoneal or intravenous injection or by oral administration, to mice that have been inoculated with a fungus, e.g., a strain of Candida albicans or Aspergillus flavus. The activity of the compounds can be assessed on the basis of the survival of a treated group of mice after the death of an untreated group of mice. The activity may be measured in terms of the dose level at which the compound provides 50% protection against the lethal effect of the infection (PD₅₀).

For human antifungal use, the compounds can be administered alone or in admixture with a pharmaceutical carrier selected in accordance with the intended route of administration and standard pharmaceutical practice. Thus, for example, they may be administered orally, parenterally, intravenously, intramuscularly or subcutaneously by means of the formulations described above in the section headed “Pharmaceutical Formulations”.

For oral and parenteral administration to human patients, the daily dosage level of the antifungal compounds of the invention can be from 0.01 to 10 mg/kg (in divided doses), depending on inter alia the potency of the compounds when administered by either the oral or parenteral route. Tablets or capsules of the compounds may contain, for example, from 5 mg. to 0.5 g of active compound for administration singly or two or more at a time as appropriate. The physician in any event will determine the actual dosage (effective amount) which will be most suitable for an individual patient and it will vary with the age, weight and response of the particular patient.

Alternatively, the antifungal compounds can be administered in the form of a suppository or pessary, or they may be applied topically in the form of a lotion, solution, cream, ointment or dusting powder. For example, they can be incorporated into a cream consisting of an aqueous emulsion of polyethylene glycols or liquid paraffin; or they can be incorporated, at a concentration between 1 and 10%, into an ointment consisting of a white wax or white soft paraffin base together with such stabilizers and preservatives as may be required.

In addition to the therapeutic uses described above, anti-fungal agents developed with such differential screening assays can be used, for example, as preservatives in foodstuff, feed supplement for promoting weight gain in livestock, or in disinfectant formulations for treatment of non-living matter, e.g., for decontaminating hospital equipment and rooms. In similar fashion, side by side comparison of inhibition of a mammalian CDK and an insect CDK, such as the Drosophilia CDK5 gene (Hellmich et al. (1994) FEBS Lett 356:317-21), will permit selection amongst the compounds herein of inhibitors which discriminate between the human/mammalian and insect enzymes. Accordingly, the present invention expressly contemplates the use and formulations of the compounds of the invention in insecticides, such as for use in management of insects like the fruit fly.

In yet another embodiment, certain of the subject CDK inhibitors can be selected on the basis of inhibitory specificity for plant CDK's relative to the mammalian enzyme. For example, a plant CDK can be disposed in a differential screen with one or more of the human enzymes to select those compounds of greatest selectivity for inhibiting the plant enzyme. Thus, the present invention specifically contemplates formulations of the subject CDK inhibitors for agricultural applications, such as in the form of a defoliant or the like.

For agricultural and horticultural purposes the compounds of the invention may be used in the form of a composition formulated as appropriate to the particular use and intended purpose. Thus the compounds may be applied in the form of dusting powders, or granules, seed dressings, aqueous solutions, dispersions or emulsions, dips, sprays, aerosols or smokes. Compositions may also be supplied in the form of dispersible powders, granules or grains, or concentrates for dilution prior to use. Such compositions may contain such conventional carriers, diluents or adjuvants as are known and acceptable in agriculture and horticulture and they are manufactured in accordance with conventional procedures. The compositions may also incorporate other active ingredients, for example, compounds having herbicidal or insecticidal activity or a further fungicide. The compounds and compositions can be applied in a number of ways, for example they can be applied directly to the plant foliage, stems, branches, seeds or roots or to the soil or other growing medium, and they may be used not only to eradicate disease, but also prophylactically to protect the plants or seeds from attack. By way of example, the compositions may contain from 0.01 to 1 wt. % of the active ingredient. For field use, likely application rates of the active ingredient may be from 50 to 5000 g/hectare.

The invention also contemplates the use of the compounds of the formula (I) and sub-groups thereof as defined herein in the control of wood decaying fingi and in the treatment of soil where plants grow, paddy fields for seedlings, or water for perfusion. Also contemplated by the invention is the use of the compounds of the formula (I) and sub-groups thereof as defined herein to protect stored grain and other non-plant loci from fungal infestation.

### EXAMPLES

The invention will now be illustrated, but not limited, by reference to the specific embodiments described in the following examples.

In the examples, the following abbreviations, where used, have the meanings shown.
AcOH acetic acid
BOC tert-butyloxycarbonyl
CDI 1,1-carbonyldiimidazole
DMAW90 Solvent mixture: DCM: MeOH, AcOH, H₂O (90:18:3:2)
DMAW120 Solvent mixture: DCM: MeOH, AcOH, H₂O (120:18:3:2)
DMAW240 Solvent mixture: DCM: MeOH, AcOH, H₂O (240:20:3:2)
DCM dichloromethane
DMF dimethylformamide
DMSO dimethyl sulphoxide
EDC 1-ethyl-3-(3′-dimethylaminopropyl)-carbodiimide
Et₃N triethylamine
EtOAc ethyl acetate
Et₂O diethyl ether
HOAt 1-hydroxyazabenzotriazole
HOBt 1-hydroxybenzotriazole
MeCN acetonitrile
MeOH methanol
P.E. petroleum ether
SiO₂ silica
TBTU N,N,N′,N′-tetramethyl-O-(benzotriazol-1-yl)uronium tetrafluoroborate
THF tetrahydrofuran

### Analytical LC-MS System and Method Description

In the examples, the compounds prepared were characterised by liquid chromatography and mass spectroscopy using the systems and operating conditions set out below. Where atoms with different isotopes are present, and a single mass quoted, the mass quoted for the compound is the monoisotopic mass (i.e. ³⁵Cl; ⁷⁹Br etc.). Several systems were used, as described below, and these were equipped with, and were set up to run under, closely similar operating conditions. The operating conditions used are also described below.

### Waters Platform LC-MS System:

| | |
|---|---|
| HPLC System: | Waters 2795 |
| Mass Spec Detector: | Micromass Platform LC |
| PDA Detector: | Waters 2996 PDA |

### Analytical Acidic Conditions:

| | |
|---|---|
| Eluent A: | H₂O (0.1% Formic Acid) |
| Eluent B: | CH₃CN (0.1% Formic Acid) |
| Gradient: | 5-95% eluent B over 3.5 minutes |
| Flow: | 0.8 ml/min |
| Column: | Phenomenex Synergi 4 μ MAX-RP 80A, 2.0 × 50 mm |

### Analytical Basic Conditions:

| | |
|---|---|
| Eluent A: | H₂O (10 mM NH₄HCO₃ buffer |
| | adjusted to pH = 9.2 with NH₄OH) |
| Eluent B: | CH₃CN |
| Gradient: | 05-95% eluent B over 3.5 minutes |
| Flow: | 0.8 ml/min |
| Column: | Phenomenex Luna C18(2) 5 μm 2.0 × 50 mm |

### Analytical Polar Conditions:

| | |
|---|---|
| Eluent A: | H₂O (0.1% Formic Acid) |
| Eluent B: | CH₃CN (0.1% Formic Acid) |
| Gradient: | 00-50% eluent B over 3 minutes |
| Flow: | 0.8 ml/min |
| Column: | Phenomenex Synergi 4 μ MAX-RP 80A, 2.0 × 50 mm |

### Analytical Lipophilic Conditions:

| | |
|---|---|
| Eluent A: | H₂O (0.1% Formic Acid) |
| Eluent B: | CH₃CN (0.1% Formic Acid) |
| Gradient: | 55-95% eluent B over 3.5 minutes |
| Flow: | 0.8 ml/min |
| Column: | Phenomenex Synergi 4 μ MAX-RP 80A, 2.0 × 50 mm |

### Analytical Long Acidic Conditions:

| | |
|---|---|
| Eluent A: | H₂O (0.1% Formic Acid) |
| Eluent B: | CH₃CN (0.1% Formic Acid) |
| Gradient: | 05-95% eluent B over 15 minutes |
| Flow: | 0.4 ml/min |
| Column: | Phenomenex Synergi 4 μ MAX-RP 80A, 2.0 × 150 mm |

### Analytical Long Basic Conditions:

| | |
|---|---|
| Eluent A: | H₂O (10 mM NH₄HCO₃ buffer |
| | adjusted to pH = 9.2 with NH₄OH) |
| Eluent B: | CH₃CN |
| Gradient: | 05-95% eluent B over 15 minutes |
| Flow: | 0.8 ml/min |
| Column: | Phenomenex Luna C18(2) 5 μm 2.0 × 50 mm |

### Platform MS Conditions:

| | |
|---|---|
| Capillary voltage: | 3.6 kV (3.40 kV on ES negative) |
| Cone voltage: | 25 V |
| Source Temperature: | 120° C. |
| Scan Range: | 100-800 amu |
| Ionisation Mode: | ElectroSpray Positive or |
| | Electro Spray Negative or |
| | ElectroSpray Positive & Negative |

### Waters Fractionlynx LC-MS System:

| | |
|---|---|
| HPLC System: | 2767 autosampler-2525 binary gradient pump |
| Mass Spec Detector: | Waters ZQ |
| PDA Detector: | Waters 2996 PDA |

### Analytical Acidic Conditions:

| | |
|---|---|
| Eluent A: | H₂O (0.1% Formic Acid) |
| Eluent B: | CH₃CN (0.1% Formic Acid) |
| Gradient: | 5-95% eluent B over 4 minutes |
| Flow: | 2.0 ml/min |
| Column: | Phenomenex Synergi 4 μ MAX-RP 80A, 4.6 × 50 mm |

### Analytical Polar Conditions:

| | |
|---|---|
| Eluent A: | H₂O (0.1% Formic Acid) |
| Eluent B: | CH₃CN (0.1% Formic Acid) |
| Gradient: | 00-50% eluent B over 4 minutes |
| Flow: | 2.0 ml/min |
| Column: | Phenomenex Synergi 4 μ MAX-RP 80A, 4.6 × 50 mm |

### Analytical Lipophilic Conditions:

| | |
|---|---|
| Eluent A: | H₂O (0.1% Formic Acid) |
| Eluent B: | CH₃CN (0.1% Formic Acid) |
| Gradient: | 55-95% eluent B over 4 minutes |
| Flow: | 2.0 ml/min |
| Column: | Phenomenex Synergi 4 μ MAX-RP 80A, 4.6 × 50 mm |

### Fractionlynx MS Conditions:

| | |
|---|---|
| Capillary voltage: | 3.5 kV (3.2 kV on ES negative) |
| Cone voltage: | 25 V (30 V on ES negative) |
| Source Temperature: | 120° C. |
| Scan Range: | 100-800 amu |
| Ionisation Mode: | ElectroSpray Positive or |
| | ElectroSpray Negative or |
| | ElectroSpray Positive & Negative |

### Mass Directed Purification LC-MS System

Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z, Lindsley C., Development of a custom high-throughput preparative liquid chromatography/mass spectrometer platform for the preparative purification and analytical analysis of compound libraries; J Comb Chem.; 2003; 5(3); 322-9.

One such system for purifying compounds via preparative LC-MS is described below although a person skilled in the art will appreciate that alternative systems and methods to those described could be used. In particular, normal phase preparative LC based methods might be used in place of the reverse phase methods described here. Most preparative LC-MS systems utilise reverse phase LC and volatile acidic modifiers, since the approach is very effective for the purification of small molecules and because the eluents are compatible with positive ion electrospray mass spectrometry. Employing other chromatographic solutions e.g. normal phase LC, alternatively buffered mobile phase, basic modifiers etc as outlined in the analytical methods described above could alternatively be used to purify the compounds.

### Preparative LC-MS Systems:

### Waters Fractionlynx System:

Hardware:

### 2767 Dual Loop Autosampler/Fraction Collector

2525 preparative pump
CFO (column fluidic organiser) for column selection
RMA (Waters reagent manager) as make up pump

### Waters ZQ Mass Spectrometer

Waters 2996 Photo Diode Array detector

### Waters ZQ Mass Spectrometer

Software:

### Masslynx 4.0

Waters MS Running Conditions:

| | |
|---|---|
| Capillary voltage: | 3.5 kV (3.2 kV on ES Negative) |
| Cone voltage: | 25 V |
| Source Temperature: | 120° C. |
| Multiplier: | 500 V |
| Scan Range: | 125-800 amu |
| Ionisation Mode: | ElectroSpray Positive or |
| | ElectroSpray Negative |

### Agilent 1100 LC-MS Preparative System:

Hardware:

Autosampler: 1100 series “prepALS”
Pump: 1100 series “PrepPump” for preparative flow gradient and 1100 series “QuatPump” for pumping modifier in prep flow
UV detector: 1100 series “MWD” Multi Wavelength Detector
MS detector: 1100 series “LC-MSD VL”

### Fraction Collector: 2×“Prep-FC”

Make Up pump: “Waters RMA”

### Agilent Active Splitter

Software:

### Chemstation: Chem32

Agilent MS Running Conditions:

| | |
|---|---|
| Capillary voltage: | 4000 V (3500 V on ES Negative) |
| Fragmentor/Gain: | 150/1 |
| Drying gas flow: | 13.0 L/min |
| Gas Temperature: | 350° C. |
| Nebuliser Pressure: | 50 psig |
| Scan Range: | 125-800 amu |
| Ionisation Mode: | ElectroSpray Positive or |
| | ElectroSpray Negative |

### Chromatographic Conditions:

Columns:

### 1. Low pH Chromatography:

Phenomenex Synergy MAX-RP, 10μ, 100×21.2 mm (alternatively used Thermo Hypersil-Keystone HyPurity Aquastar, 5μ, 100×21.2 mm for more polar compounds)

### 2. High pH Chromatography:

### Phenomenex Luna C18 (2), 10μ, 100×21.2 mm

(alternatively used Phenomenex Gemini, 5μ, 100×21.2 mm)

Eluents:

### 1. Low pH Chromatography:

Solvent A: H₂O+0.1% Formic Acid, pH˜1.5

### Solvent B: CH₃CN+0.1% Formic Acid

### 2. High pH Chromatography:

Solvent A: H₂O+10 mM NH₄HCO₃+NH4OH, pH=9.2

### Solvent B: CH₃CN

### 3. Make Up Solvent:

MeOH+0.2% Formic Acid (for both chromatography type)

Methods:

According to the analytical trace the most appropriate preparative chromatography type was chosen. A typical routine was to run an analytical LC-MS using the type of chromatography (low or high pH) most suited for compound structure. Once the analytical trace showed good chromatography a suitable preparative method of the same type was chosen. Typical running condition for both low and high pH chromatography methods were:

Flow rate: 24 ml/min
Gradient: Generally all gradients had an initial 0.4 min step with 95% A+5% B. Then according to analytical trace a 3.6 min gradient was chosen in order to achieve good separation (e.g. from 5% to 50% B for early retaining compounds; from 35% to 80% B for middle retaining compounds and so on)
Wash: 1.2 minute wash step was performed at the end of the gradient
Re-equilibration: 2.1 minutes re-equilibration step was ran to prepare the system for the next run
Make Up flow rate: 1 ml/min

Solvent:

All compounds were usually dissolved in 100% MeOH or 100% DMSO

From the information provided someone skilled in the art could purify the compounds described herein by preparative LC-MS.

The starting materials for each of the Examples are commercially available unless otherwise specified.

### Example 1

### Synthesis of 4-(2,6-Dichloro-benzoylamino)-isothiazole-3-carboxylic acid (1-methyl-piperidin-4-yl)-amide

### 1A. Synthesis of 4-Amino-isothiazole-3-carboxylic acid methyl ester

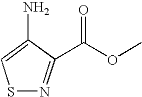

Thionyl chloride (0.601 g, 5.05 mmol) was added dropwise at 0° C. to a solution of 4-amino-isothiazole-3-carboxylic acid (0.485 g, 3.37 mmol) in methanol (10 ml) and the mixture was stirred for 20 h at ambient temperature. The reaction mixture was reduced in vacuo and dried through azeotrope with toluene to afford 4-amino-isothiazole-3-carboxylic acid methyl ester as a pale yellow solid (0.463 g, 87%). (LC/MS: Rₜ 1.65, [M+H]⁺ 159.08).

### 1B. Synthesis of 4-(2,6-Dichloro-benzoylamino)-isothiazole-3-carboxylic acid methyl ester

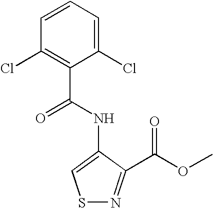

2,6-Dichloro-benzoyl chloride (0.332 g, 1.59 mmol) and triethylamine (0.176 g, 0.174 mmol) were added to a solution of 4-amino-isothiazole-3-carboxylic acid methyl ester (0.231 g, 1.46 mmol) in THF (5 ml) and the resulting suspension was stirred at ambient temperature for 16 h. The reaction mixture was reduced in vacuo and the residue partitioned between ethyl acetate (50 ml) and water (50 ml) and the aqueous phase back extracted with ethyl acetate (50 ml). The combined organics were washed with brine (50 ml), dried (MgSO₄) and reduced in vacuo. The resulting residue was subjected to column chromatography eluting with a 0-40% gradient of ethyl acetate in petroleum ether. Product containing fractions were reduced in vacuo to give 4-(2,6-dichloro-benzoylamino)-isothiazole-3-carboxylic acid methyl ester as a yellow solid. (0.17 g, 36%) (LC/MS: Rₜ 3.09, [M+H]⁺ 330.93).

### 1C. Synthesis of 4-(2,6-Dichloro-benzoylamino)-isothiazole-3-carboxylic acid

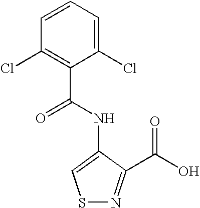

A mixture of 4-(2,6-dichloro-benzoylamino)-isothiazole-3-carboxylic acid methyl ester (0.173 g, 0.52 mmol) in 2 M aqueous NaOH/dioxane (1:1, 6 ml) was stirred at ambient temperature for 16 h. Volatile materials were removed in vacuo, water (50 ml) was added and the mixture taken to pH 4 by the addition of 1M aqueous HCl. The resultant precipitate was collected by filtration and dried under suction to give 4-(2,6-dichloro-benzoylamino)-isothiazole-3-carboxylic acid as a white solid (0.134 g, 82%).

### 1D. Synthesis of 4-(2,6-Dichloro-benzoylamino)-isothiazole-3-carboxylic acid (1-methyl-piperidin-4-yl)-amide

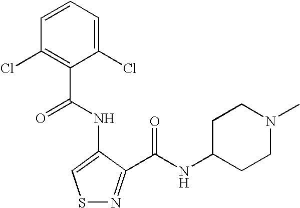

4-(2,6-Dichloro-benzoylamino)-isothiazole-3-carboxylic acid (0.134 g, 0.42 mmol) was combined with 1-methyl-piperidin-4-ylamine (0.057 g, 0.50 mmol), EDC (0.096 g, 0.50 mmol), HOBt (0.067 g, 0.50 mmol) and DMF (6 ml) and the resulting reaction mixture was stirred at ambient temperature for 18 hours. The reaction mixture was reduced in vacuo and the residue partitioned between ethyl acetate (50 ml) and saturated aqueous sodium bicarbonate solution (50 ml). The organic layer was washed with brine, dried (MgSO₄), reduced in vacuo and triturated with diethyl ether to give 4-(2,6-dichloro-benzoylamino)-isothiazole-3-carboxylic acid (1-methyl-piperidin-4-yl)-amide as a white solid (0.112 g, 65%). (LC/MS: Rₜ 2.06, [M+H]⁺ 413.04).

### Example 2

### Synthesis of 5-(2,6-difluoro-benzoylamino)-thiazole-4-carboxylic acid piperidin-4-ylamide

### 2A. Synthesis of 5-(4-methoxy-benzylamino)-thiazole-4-carboxylic acid ethyl ester

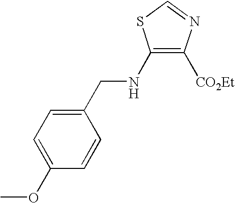

To a vigorously stirred solution of potassium tert-butoxide (5.45 g, 48.59 mmoles) in THF (140 ml) was added dropwise ethyl isocyanoacetate (4.8 ml, 44.17 mmoles). The suspension was stirred at ambient temperature for 10 minutes. To the suspension was added dropwise 4-methoxybenzyl isothiocyanate (6.9 ml, 44.17 mmoles). The suspension was stirred at ambient temperature for a further 2 hours. Acetic acid (10 ml) was added to the suspension and then the solvent was removed in vacuo. The residue was partitioned between EtOAc and water. The organic portion was dried (MgSO₄), filtered and evaporated in vacuo. The residue was purified [Biotage SP4, 2×40M, flow rate 40 ml/min, gradient 1:4 EtOAc/Petrol to 7:3 EtOAc/Petrol] to give 5-(4-methoxy-benzylamino)-thiazole-4-carboxylic acid ethyl ester as a brown oil (7.6 g, 59%). (LC/MS: Rₜ 2.90, [M+H]⁺ 292.99).

### 2B. Synthesis of 5-[2,6-difluoro-benzoyl)-(4-methoxy-benzyl)-amino]-thiazole-4-carboxylic acid ethyl ester

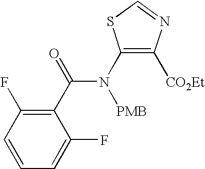

To a stirred solution of 5-(4-methoxy-benzylamino)-thiazole-4-carboxylic acid ethyl ester (1.0 g, 3.42 mmoles) in DMF (10 ml) was added portionwise sodium hydride (301 mg, 7.53 mmoles). The solution was stirred at ambient temperature for 10 minutes. To the reaction mixture was added 2,6-difluorobenzoyl chloride (0.858 ml, 6.84 mmoles), and then stirred at ambient temperature for 1 hour. The reaction mixture was partitioned between ether and water. The organic portion was dried (MgSO₄), filtered and evaporated in vacuo. The residue was purified [Biotage SP4, 40S, flow rate 40 ml/min, gradient 1:4 EtOAc/Petrol to 7:3 EtOAc/Petrol) to give 5-[2,6-difluoro-benzoyl)-(4-methoxy-benzyl)-amino]-thiazole-4-carboxylic acid ethyl ester as a white solid (1.1 g, 74%). (LC/MS: Rₜ 3.16, [M+H]⁺ 432.98).

### 2C. Synthesis of 5-[(2,6-difluoro-benzoyl)-(4-methoxy-benzyl)-amino]-thiazole-4-carboxylic acid

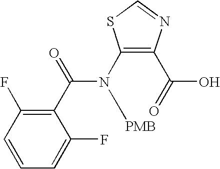

A solution of 5-[2,6-difluoro-benzoyl)-(4-methoxy-benzyl)-amino]-thiazole-4-carboxylic acid ethyl ester (1.1 g, 2.55 mmoles) in a mixture of ethanol (20 ml) and 2N sodium hydroxide solution (20 ml) was stirred at ambient temperature for 24 hours. Ethanol was evaporated in vacuo. The residue was partitioned between EtOAc and 2N hydrochloric acid. The organic portion was dried (MgSO₄), filtered and evaporated in vacuo to give 5-[(2,6-difluoro-benzoyl)-(4-methoxy-benzyl)-amino]-thiazole-4-carboxylic acid as a pale yellow solid (0.95 g, 92%). (LC/MS: Rₜ 2.68, [M+H]⁺ 404.92).

### 2D. Synthesis of 4-({5-[(2,6-difluoro-benzoyl)-(4-methoxy-benzyl)-amino]-thiazole-4-carbonyl}-amino)-piperidine-1-carboxylic acid tert-butyl ester

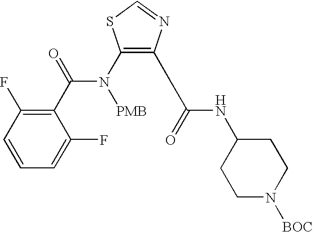

A solution of 5-[(2,6-difluoro-benzoyl)-(4-methoxy-benzyl)-amino]-thiazole-4-carboxylic acid (from Example 2C) (350 mg, 0.87 mmoles), 4-amino-piperidine-1-carboxylic acid tert-butyl ester (174 mg, 0.87 mmoles), EDC (200 mg, 1.04 mmoles), and HOBt (140 mg, 1.04 mmoles) in dichloromethane (10 ml) was stirred at ambient temperature for 3 hours. The reaction mixture was diluted with EtOAc, and washed with a saturated solution of NaHCO₃ and then brine. The organic portion was dried (MgSO₄), filtered and evaporated in vacuo to give 4-({5-[(2,6-difluoro-benzoyl)-(4-methoxy-benzyl)-amino]-thiazole-4-carbonyl}-amino)-piperidine-1-carboxylic acid tert-butyl ester as a viscous pale yellow oil (460 mg, 90%). (LC/MS: Rt 3.42, [M+H]+ 587.15).

### 2E. Synthesis of 5-(2,6-difluoro-benzoylamino)-thiazole-4-carboxylic acid piperidin-4-ylamide

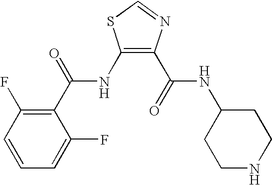

A solution of 4-({5-[(2,6-difluoro-benzoyl)-(4-methoxy-benzyl)-amino]-thiazole-4-carbonyl}-amino)-piperidine-1-carboxylic acid tert-butyl ester (460 mg, 0.78 mmoles) in trifluoroacetic acid (2 ml) and anisole (170 μl, 1.56 mmoles) was heated at 100° C. (80 W) in a CEM discover microwave synthesizer for 10 minutes.

The solvent was removed in vacuo. The residue was purified by trituration with ether to give 5-(2,6-difluoro-benzoylamino)-thiazole-4-carboxylic acid piperidin-4-ylamide as a white solid (279 mg, 98%). (LC/MS: Rₜ 1.95, [M+H]⁺ 366.97).

### Examples 3 to 5

Be following the methods described in Examples 1 and 2, the compounds shown in the table below were prepared.

| Example | Structure | Method of Preparation | LCMS |
|---|---|---|---|
| 3 | 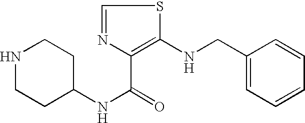 | As example 2, but usedbenzyl chloride instead of2,6-difluorobenzoyl chloridein step 2B | [M + H]⁺317.25Rₜ 2.01 |
| 4 | 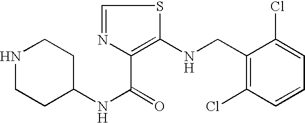 | As example 2, but using2,6-dichlorobenzyl chlorideinstead of 2,6-difluorobenzoyl chloridestep 2B | [M + H]⁺385.20Rₜ 2.16 |
| 5 | 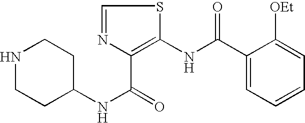 | As example 2, but using 2-ethoxybenzoyl chlorideinstead of 2,6-difluorobenzoyl chloride instep 2B | [M + H]⁺375.28Rₜ 2.30 |

### Example 6

Synthesis of 5-(1-phenyl-ethylamino)-thiazole-4-carboxylic acid piperidin-4-ylamide

### 6A. Synthesis of 5-(1-phenyl-ethylamino)-thiazole-4-carboxylic acid ethyl ester

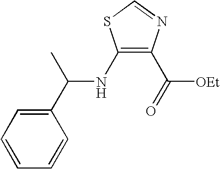

To a vigorously stirred solution of potassium tert-butoxide (1.4 g, 12.27 mmoles) in THF (100 ml) was added dropwise ethyl isocyanoacetate (1.3 ml, 12.27 mmoles). The suspension was stirred at ambient temperature for 10 minutes. A solution of α-methyl benzyl isothiocyanate (2 g, 12.27 mmoles) in THF (100 ml) was added dropwise to the reaction mixture. The suspension was stirred at ambient temperature for a further 2 hours. Acetic acid (10 ml) was added to the suspension and then the solvent was removed in vacuo. The residue was partitioned between EtOAc and water. The organic portion was washed with brine, dried (MgSO₄), filtered and evaporated in vacuo. The residue was purified by flash chromatography (Biotage SP4, 40S, flow rate 40 ml/min, gradient 3:17 EtOAc/Petrol to 3:2 EtOAc/Petrol) to give 5-(1-phenyl-ethylamino)-thiazole-4-carboxylic acid ethyl ester as a yellow oil (900 mg, 27%). (LC/MS: Rₜ 3.06, [M+H]⁺ 277.25).

### 6B. Synthesis of 5-(1-phenyl-ethylamino)-thiazole-4-carboxylic acid

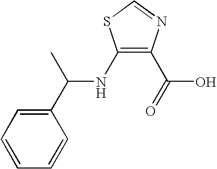

A solution of 5-(1-phenyl-ethylamino)-thiazole-4-carboxylic acid ethyl ester (900 mg, 3.26 mmoles) in a mixture of ethanol (20 ml) and 2N sodium hydroxide solution (20 ml) was stirred at ambient temperature for 24 hours. The ethanol was removed in vacuo and the residue partitioned between EtOAc and 2N HCl. The organic portion was dried (MgSO₄), filtered and evaporated in vacuo to give 5-(1-phenyl-ethylamino)-thiazole-4-carboxylic acid as a white solid (580 mg, 72%). (LC/MS: Rₜ 2.58, [M+H]⁺ 249.15).

### 6C. Synthesis of 5-(1-phenyl-ethylamino)-thiazole-4-carboxylic acid piperidin-4-ylamide

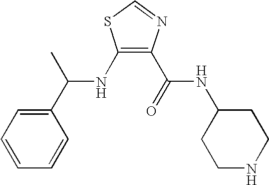

A solution of 5-(1-phenyl-ethylamino)-thiazole-4-carboxylic acid (580 mg, 2.34 mmoles), 4-amino-piperidine-1-carboxylic acid tert-butyl ester (468 mg, 2.34 mmoles), EDC (538 mg, 2.81 mmoles) and HOBt (379 mg, 2.81 mmoles) in dichloromethane (10 ml) was stirred at ambient temperature for 3 hours. The reaction mixture was diluted with EtOAc, washed with a saturated solution of sodium hydrogen carbonate and then brine. The organic portion was dried (MgSO₄), filtered and evaporated in vacuo. The residue was purified by flash chromatography (Biotage SP4, 40M, flow rate 40 ml/min, gradient 3:17 EtOAc/Petrol to 3:2 EtOAc/Petrol) to give a colourless oil. The oil was dissolved in HCl in dioxane (4M, 10 ml) and stirred at ambient temperature for 72 hours. The reaction mixture was azeotroped with toluene/methanol in vacuo. The residue was triturated with ether and filtered to give 5-(1-phenyl-ethylamino)-thiazole-4-carboxylic acid piperidin-4-ylamide as a light green dihydrochloride salt (630 mg, 67%) (LC/MS: Rₜ 2.09, [M+H]⁺ 331.25).

### Examples 7 & 8

By following the methods described in Example 6, modified where indicated in the table below, the compounds of Examples 7 and 8 were prepared.

| Example | Structure | Method of Preparation | LCMS |
|---|---|---|---|
| 7 | 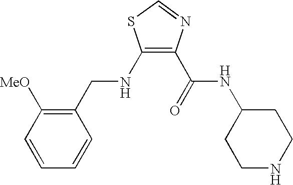 | As example 6, but using 2-methoxy benzylisothiocyanate instead ofα-methyl benzylisothiocyanate in step 6A | [M + H]⁺347.27Rₜ 2.11 |
| 8 | 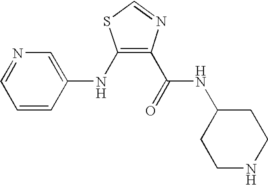 | As example 6, but using 3-pyridyl isothiocyanateinstead of α-methyl benzylisothiocyanate in step 6A | [M + H]⁺304.24Rₜ 2.49 |

### Example 9

### Synthesis of 4-(2,6-dichloro-benzoylamino)-isothiazole-3-carboxylic acid (4-piperazin-1-yl-phenyl)-amide hydrochloride

### 9A. Synthesis of 4-(2,6-dichloro-benzoylamino)-isothiazole-3-carboxylic acid

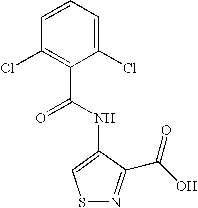

To a solution of 4-amino-isothiazole-3-carboxylic acid (500 mg; 3.5 mmol) in DCM (25 ml) was added triethylamine (1.06 ml; 2.2 equiv.) followed by 2,6-dichlorobenzoyl chloride (550 μl; 1.1 equiv.) and the mixture was then stirred at room temperature for 16 hours. After this time, the reaction mixture was diluted with DCM, washed with 2M hydrochloric acid followed by brine, dried (MgSO₄), filtered and evaporated to give a gummy white solid. The solid was triturated with 1:1 saturated sodium hydrogen carbonate and diethyl ether, the solid was collected by filtration, washed with water and sucked dry to give 580 mg of 4-(2,6-dichloro-benzoylamino)-isothiazole-3-carboxylic acid as a white solid. ¹H NMR (d₆-DMSO) 13.2 (1H, bs), 9.2 (1H, s), 7.7-7.5 (4H, m).

### 9B. Synthesis of 4-(2,6-dichloro-benzoylamino)-isothiazole-3-carboxylic acid (4-piperazin-1-yl-phenyl)-amide hydrochloride

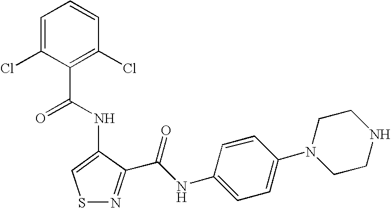

To a solution of 4-(2,6-dichloro-benzoylamino)-isothiazole-3-carboxylic acid (75 mg; 0.24 mmol) in DMF (5 ml) was added HOBt (40 mg; 1.2 equiv.), EDAC (55 mg; 1.2 equiv.) triethylamine (80 ul; 2.2 equiv.) and 4-(4-amino-phenyl)-piperazine-1-carboxylic acid tert-butyl ester dihydrochloride (91 mg; 1.1 equiv.). The reaction mixture was stirred at room temperature overnight and then poured onto saturated sodium hydrogen carbonate. The resulting solid was collected by filtration, washed with water and sucked dry. The crude material was dissolved in saturated HCl/EtOAc (10 ml), stirred at room temperature overnight and then evaporated before purifying by flash column chromatography (eluting with DMAW120), followed by prep. LC/MS. The hydrochloride salt was formed by evaporation from HCl/EtOAc and 8 mg of 4-(2,6-dichloro-benzoylamino)-isothiazole-3-carboxylic acid (4-piperazin-1-yl-phenyl)-amide hydrochloride was isolated as a white solid. ¹H NMR (d₆-DMSO) 11.05 (1H, s), 10.75 (1H, s), 9.55 (1H, s), 7.70-7.55 (5H, m), 7.00 (2H, d), 3.35-3.20 (8H, m).

### Example 10

### Synthesis of 5-(2,4,6-trichloro-benzoylamino)-thiazole-4-carboxylic acid phenylamide

### 10A. 5-(2,4,6-Trichloro-benzoylamino)-thiazole-4-carboxylic acid

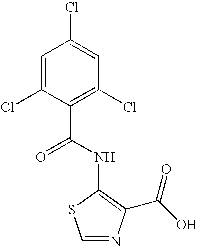

A mixture of 5-[(4-methoxy-benzyl)-(2,4,6-trichloro-benzoyl)-amino]-thiazole-4-carboxylic acid ethyl ester* (595 mg) and anisole (300 ml) in trifluoroacetic acid (5 ml) was heated at 120° C. for 30 minutes in a CEM explorer microwave synthesiser and then evaporated. The residue was partitioned between EtOAc and saturated sodium hydrogen carbonate solution, the EtOAc layer was separated, dried (MgSO₄) and evaporated to give 5-(2,4,6-trichloro-benzoylamino)-thiazole-4-carboxylic acid ethyl ester as a yellow/brown gum. The crude material was dissolved in methanol (10 ml), treated with 2M sodium hydroxide solution and heated at 65° C. for 16 hours. The methanol was evaporated in vacuo and the aqueous layer acidified with 2M HCl. The solid was collected by filtration, washed with water and sucked dry to give 365 mg of 5-(2,4,6-trichloro-benzoylamino)-thiazole-4-carboxylic acid as an off-white solid. (LC/MS: Rₜ 2.87, [M+H]⁺ 351). *Prepared in a manner analogous to Example 2B.

### 10B. 5-(2,4,6-trichloro-benzoylamino)-thiazole-4-carboxylic acid phenylamide

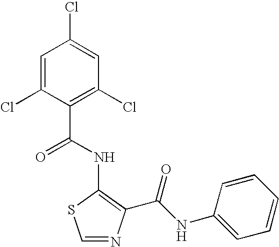

A solution of 5-(2,4,6-trichloro-benzoylamino)-thiazole-4-carboxylic acid (50 mg; 0.143 mmol), aniline (15 μl; 1.1 equiv.), HOBt (25 mg; 1.2 equiv.) and EDAC (35 mg; 1.2 equiv.) in 5 ml of DMF was stirred at room temperature overnight then evaporated. The residue was dissolved in EtOAc, washed with 2M HCl followed by saturated sodium hydrogen carbonate solution, then dried (gSO₄) and evaporated. The crude material was triturated with petroleum ether, filtered and dried under suction to give 31 mg of 5-(2,4,6-trichloro-benzoylamino)-thiazole-4-carboxylic acid phenylamide as a beige solid. ¹H NMR (d₆-DMSO) 12.05 (1H, s), 10.45 (1H, s), 8.90 (1H, s), 7.85 (2H, s) 7.80 (2H, d), 7.45 (2H, d), 7.15 (1H, t).

### Example 11

### 5-(2,4,6-trichloro-benzoylamino)-thiazole-4-carboxylic acid (5-piperazin-1-yl-pyridin-2-yl)-amide

### 11A. 5-(2,4,6-trichloro-phenyl)-3a,7a-dihydro-6-oxa-3-thia-1,4-diaza-inden-7-one

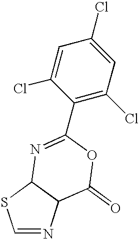

Oxalyl chloride (130 μl; 1.5 equiv.) was added to a stirred suspension of 5-(2,4,6-trichloro-benzoylamino)-thiazole-4-carboxylic acid (350 mg; 1 mmol) in 99:1 DCM/DMF (20 ml) and stirred at room temperature for 72 hours. The reaction mixture was evaporated and re-evaporated with toluene (×2) to give 330 mg of 5-(2,4,6-trichloro-phenyl)-3a,7a-dihydro-6-oxa-3-thia-1,4-diaza-inden-7-one. (LC/MS: Rₜ 3.41, [M+H]⁺ 333).

### 11C. 4-(6-amino-pyridin-3-yl)-piperazine-1-carboxylic acid tert-butyl ester

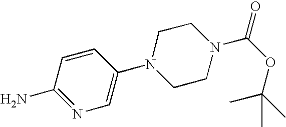

The title compound was prepared by the method described in J. Med. Chem. 2005, 48 (7), 2388-2406. Thus, a mixture of 5-bromo-2-nitropyridine (5.03 g; 24.8 mmol), potassium carbonate (3.8 g; 1.1 equiv.), piperazine (2.8 g; 1.3 equiv.) and tetra-n-butylammonium iodide (0.46 g; 0.05 equiv.) in 60 ml of DMSO was heated at 80° C. overnight, then cooled and poured into 300 ml of water. The solid was collected by filtration washed with water (50 ml) and DCM (50 ml) and sucked dry to give 650 mg of yellow solid. The aqueous filtrate was extracted with CHCl₃ (4×150 ml), the combined organics were washed with brine (100 ml), dried (MgSO₄), filtered and evaporated to give 5.5 g of yellow solid.

The 2 batches of product were dissolved in THF/water (40 ml:10 ml), treated with sodium hydrogen carbonate (3.1 g; 1.5 equiv.) and di-tert-butyl dicarbonate (6.5 g; 1.2 equiv.), stirred at room temperature overnight and then evaporated. The residue was partitioned between DCM and brine (100 ml:100 ml), and the DCM layer separated, dried (MgSO₄), filtered and evaporated. The crude material was purified by flash column chromatography (eluting with 1:2 to 2:1 EtOAc/P.E.). Product-containing fractions were combined and evaporated to give 4.5 g of 4-(6-nitro-pyridin-3-yl)-piperazine-1-carboxylic acid tert-butyl ester.

A mixture of 4-(6-nitro-pyridin-3-yl)-piperazine-1-carboxylic acid tert-butyl ester and 10% palladium on carbon in ethanol/ethyl acetate (100 ml/100 ml) was hydrogenated at room temperature and pressure overnight, then filtered and the filtrate evaporated to give to give 4 g of 4-(6-amino-pyridin-3-yl)-piperazine-1-carboxylic acid tert-butyl ester as a brown solid. ¹H NMR (d₆-DMSO) 7.63 (1H, d), 7.18 (1H, dd), 6.40 (1H, d), 5.45 (2H, s), 3.45 (4H, m) 2.85 (4H, m), 1.43 (9H, s).

### 11C. 4-(6-{[5-(2,4,6-trichloro-benzoylamino)-thiazole-4-carbonyl]-amino}-pyridin-3-yl)-piperazine-1-carboxylic acid tert-butyl ester

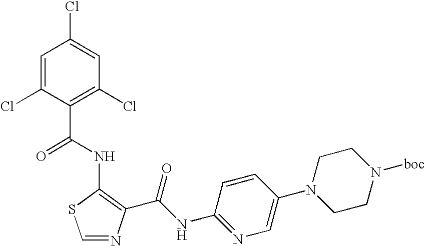

A mixture of 5-(2,4,6-trichloro-phenyl)-3a,7a-dihydro-6-oxa-3-thia-1,4-diaza-inden-7-one (100 mg; 0.3 mmol) and 4-(6-amino-pyridin-3-yl)-piperazine-1-carboxylic acid tert-butyl ester (420 mg; 5 equiv.) in 4 ml of NMP was heated at 150° C. for 30 minutes in a CEM explorer microwave synthesiser. The reaction mixture was partitioned between EtOAc and saturated NH₄Cl solution, and the EtOAc layer was separated, washed with saturated NH₄Cl solution, dried (MgSO₄) and evaporated. The crude material was purified by flash chromatography (1:2 EtOAc/Petrol) to give 90 mg of 4-(6-{[5-(2,4,6-trichloro-benzoylamino)-thiazole-4-carbonyl]-amino}-pyridin-3-yl)-piperazine-1-carboxylic acid tert-butyl ester as a cream solid. (LC/MS: Rₜ 4.05, [M+H]⁺ 611).

### 11D. 5-(2,4,6-trichloro-benzoylamino)-thiazole-4-carboxylic acid (5-piperazin-1-yl-pyridin-2-yl)-amide

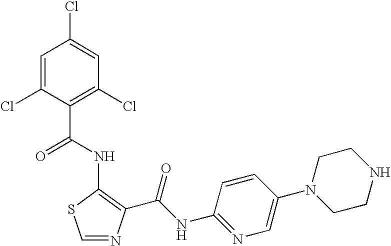

4-(6-{[5-(2,4,6-Trichloro-benzoylamino)-thiazole-4-carbonyl]-amino}-pyridin-3-yl)-piperazine-1-carboxylic acid tert-butyl ester (90 mg) was dissolved in saturated HCl/EtOAc (10 ml) and stirred at room temperature overnight. The precipitate was collected by filtration, washed with EtOAc and then dried in vacuo to give 30 mg of 5-(2,4,6-trichloro-benzoylamino)-thiazole-4-carboxylic acid (5-piperazin-1-yl-pyridin-2-yl)-amide hydrochloride as a yellow solid. ¹H NMR (d₆-DMSO) 11.94 (1H, s), 10.25 (1H, s), 9.25 (2H, br s), 8.90 (1H, s), 8.18 (1H, s) 8.10 (1H, d), 7.85 (2H, s), 7.70 (1H, d), 3.45 (4H, m), 3.20 (4H, m). (LC/MS: Rₜ 2.42, [M+H]⁺ 511).

### Example 12

### 5-(2,4,6-trichloro-benzoylamino)-thiazole-4-carboxylic acid [1-(tetrahydro-pyran-4-yl)-piperidin-4-yl]-amide

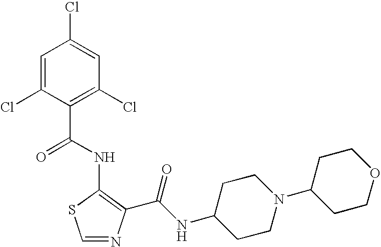

A mixture of 5-(2,4,6-trichloro-benzoylamino)-thiazole-4-carboxylic acid piperidin-4-ylamide (100 mg; 0.23 mmol) tetrahydro-4H-pyran-1-one (35 mg; 2 equiv.) and sodium cyanoborohydride (45 mg; 3 equiv.) in DCM (10 ml) was stirred at room temperature overnight. The DCM was evaporated, replaced by 10 ml of toluene a catalytic amount of p-toluenesulphonic acid was added, heated at reflux overnight then evaporated. The residue was dissolved in EtOAc, washed with saturated sodium hydrogen carbonate solution, then dried (MgSO₄) and evaporated. Purification by preparative LC/MS gave 5 mg of 5-(2,4,6-trichloro-benzoylamino)-thiazole-4-carboxylic acid [1-(tetrahydro-pyran-4-yl)-piperidin-4-yl]-amide as a white solid. ¹H NMR (d₄-MeOH) 8.50 (1H, s), 7.50 (2H, s), 3.95 (2H, dd) 3.88 (1H, m), 3.35 (2H, t), 3.05 (2H, m), 2.65 (1H, m), 2.45 (2H, m), 2.00-1.50 (8H, m). (LC/MS: Rₜ 2.33, [M+H]⁺ 517).

| Example | | Chemical | Method of | | |
|---|---|---|---|---|---|
| No. | Chemical structure | name | preparation | NMR data | LC/MS |
| 13 | 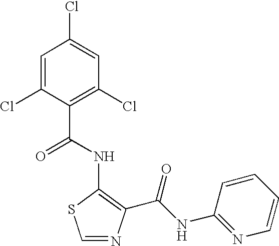 | 5-(2,4,6-trichloro-benzoylamino)-thiazole-4-carboxylic acidpyridin-2-ylamide | Example 10using 2-aminopyridinein step B. | ¹H NMR (d₆-DMSO) 11.98(1H, s), 9.88 (1H,s), 8.90 (1H, s),8.40 (1H, d) 8.15(1H, d), 7.85-7.95 (3H, m),7.20 (1H, dd). | |
| 14 | 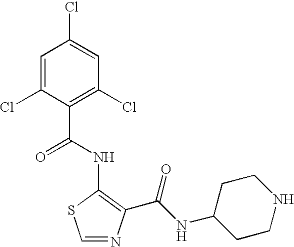 | 5-(2,4,6-trichloro-benzoylamino)-thiazole-4-carboxylic acidpiperidin-4-ylamidehydrochloride | Example 10using 4-amino-piperidine-1-carboxylicacid tert-butylester in step Bfollowed bydeprotectionusingsaturated HCl/EtOAc. | ¹H NMR (d₆-DMSO) 12.10(1H, s), 9.02 (1H,br s), 8.85 (1H,d), 8.80 (1H, s)8.65 (1H, br s),7.90 (2H, s), 4.05(1H, m), 3.30(2H, d), 2.95(2H, dd), 2.80(2H, t), 1.83 (2H,dd), 1.95-1.75(4H, m). | Rₜ 2.26,[M + H]⁺433 |
| 15 | 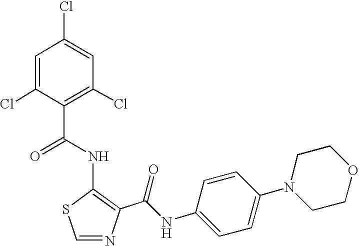 | 5-(2,4,6-trichloro-benzoylamino)-thiazole-4-carboxylic acid(4-morpholin-4-yl-phenyl)-amide | Example 10using 4-morpholin-4-yl-phenyl-amine in step B. | ¹H NMR (d₄-MeOH) 7.70(1H,s), 6.80 (2H, m),6.65 (2H, s), 6.75(2H, m), 3.05(4H, m), 2.35(4H, m). | Rₜ 3.66,[M + H]⁺511 |
| 16 | 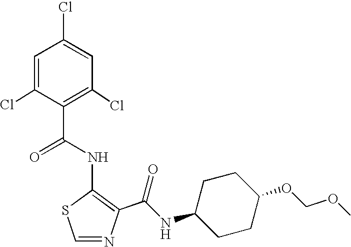 | 5-(2,4,6-trichloro-benzoylamino)-thiazole-4-carboxylic acid(4-methoxymethoxy-cyclohexyl)-amide | Example 10trans-4-methoxy-methoxy-cyclohexyl-amine in step B. | ¹H NMR(d₄-MeOH) 8.55(1H, s), 7.55 (2H,s), 4.65 (2H, s),3.9-3.8 (1H, m),3.6-3.5 (1H, m),3.3 (1H, s), 2.15-1.95 (4H, m)1.55-1.35 (4H,m). | Rₜ 3.68,[M + H]⁺492 |
| 17 | 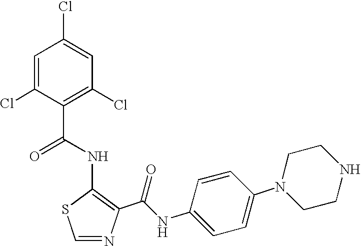 | 5-(2,4,6-trichloro-benzoylamino)-thiazole-4-carboxylic acid(4-piperazin-1-yl-phenyl)-amide aceticacid salt | Example 11using 4-(4-amino-phenyl)-piperazine-1-carboxylicacid tert-butylester di-hydrochloridein step B. | ¹H NMR (d₆-DMSO) 8.50-8.35 (1H, br s),7.75 (2H, s), 7.50-7.35 (2H, m),6.90 (2H, d),3.25 (4H, m),3.15 (4H, m). | Rₜ 2.52,[M + H]⁺510 |
| 18 | 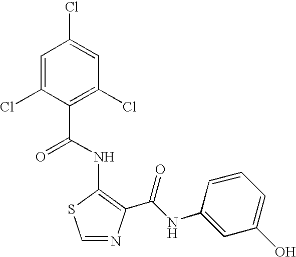 | 5-(2,4,6-trichloro-benzoylamino)-thiazole-4-carboxylic acid(3-hydroxy-phenyl)-amide | Example 11using 3-aminophenolin step B. | ¹H NMR (d₆-DMSO) 12.00(1H, s), 10.25(1H, s), 9.40 (1H,s), 8.85 (1H, s),7.85 (2H, d,)7.35 (1H, s), 7.20(1H, d), 7.10(1H, t), 6.52 (1H,d). | Rₜ 3.47,[M + H]⁺442 |
| 19 | 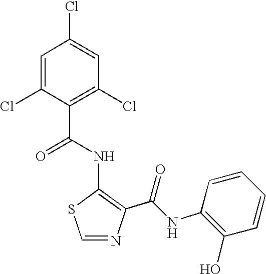 | 5-(2,4,6-trichloro-benzoylamino)-thiazole-4-carboxylic acid(2-hydroxy-phenyl)-amide | Example 11,using 3-aminophenolin step B. | ¹H NMR (d₆-DMSO) 11.95(1H, s), 10.30(1H, s), 9.30 (1H,s), 8.90 (1H, s),8.22 (1H, d) 7.87(2H, s), 6.95 (2H,m), 6.82 (1H, t). | Rₜ 3.71,[M + H]⁺442 |
| 20 | 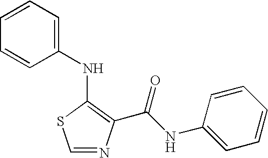 | 5-phenylamino-thiazole-4-carboxylic acidphenylamide | Example 6,using phenyl-isothiocyanatein step A andaniline in stepC. | ¹H NMR (d₆-DMSO) 10.35(1H, s), 10.05(1H, s), 8.45 (1H,s), 7.85 (2H, d),7.45 (2H, m)7.35 (4H, m),7.10(2H, m). | Rₜ 3.63,[M + H]⁺296 |
| 21 | 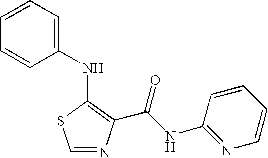 | 5-phenylamino-thiazole-4-carboxylic acidpyridin-2-yl-amide | Example 6,using phenyl-isothiocyanatein step A and2-amino-pyridine instep C. | ¹H NMR (d₆-DMSO) 10.10(1H, s), 9.50 (1H,s), 8.45 (1H, s),8.35 (1H, d),8.25 (1H, d),7.85 (1H, t), 7.50-7.30 (4H, m)7.20 (2H, m). | Rₜ 3.05,[M + H]⁺297 |
| 22 | 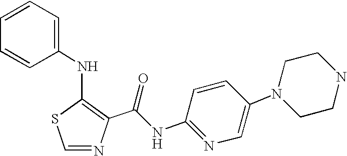 | 5-phenylamino-thiazole-4-carboxylic acid(5-piperazin-1-yl-pyridin-2-yl)-amidehydrochloride | Example 6,using phenyl-isothiocyanatein step A and4-(6-amino-pyridin-3-yl)-piperazine-1-carboxylicacid tert-butylester in stepC. | ¹H NMR (d₆-DMSO) 10.10(1H, s), 10.05(1H, s), 9.40 (2H,br s) 8.45 (1H, s),8.15 (2H, m),7.80 (1H, dd),7.45 (2H, t), 7.35(2H, t) 7.15 (1H,t), 3.45 (4H, m),3.25 (4H, m). | Rₜ 2.11,[M + H]⁺381 |
| 23 | 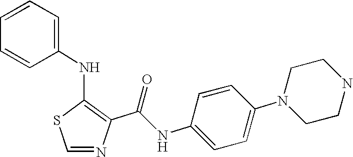 | 5-phenylamino-thiazole-4-carboxylic acid(4-piperazin-1-yl-phenyl)-amidehydrochloride | Example 6,using phenyl-isothiocyanatein step A and4-(4-amino-phenyl)-piperazine-1-carboxylicacid tert-butylester di-hydrochloridein step C. | ¹H NMR (d₆-DMSO) 10.40(1H, s), 9.88 (1Hs), 8.42 (1H, s),8.30 (1H, s), 7.70(2H, d), 7.40(2H, t), 7.35 (2H,d), 7.10 (1H, t)6.92 (2H, d),3.10 (4H, m),2.95 (4H, m). | Rₜ 2.34,[M + H]⁺380 |
| 24 | 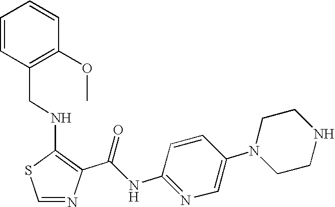 | 5-(2-methoxy-benzylamino)-thiazole-4-carboxylic acid(5-piperazin-1-yl-pyridin-2-yl)-amide aceticacid salt | Example 6,using 2-methoxy-benzyl-isothiocyanatein step A and4-(6-amino-pyridin-3-yl)-piperazine-1-carboxylicacid tert-butylester in step C. | ¹H NMR (d₆-DMSO) 9.10(1H, s), 8.20 (1H,t), 8.15 (1H, s),8.05 (1H, d),8.02 (1H, d) 7.45(1H, dd), 7.30(2H, m), 7.05(1H, d) 6.95 (1H,t), 3.85 (1H, t),4.45 (2H, d),3.12 (4H, m),2.92 (4H, m),1.90 (3H, s). | Rₜ 2.17,[M + H]⁺425 |
| 25 | 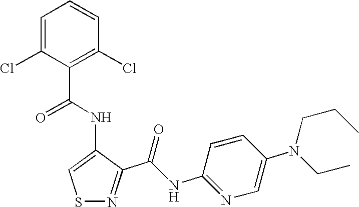 | 4-(2,6-dichloro-benzoylamino)-isothiazole-3-carboxylic acid(5-piperazin-1-yl-pyridin-2-yl)-amide | Example 9,using 4-(6-amino-pyridin-3-yl)-piperazine-1-carboxylicacid tert-butylester in step B. | ¹H NMR (d₆-DMSO) 10.92(1H, br s), 9.55(1H, s), 8.10 (1H,d) 7.88 (1H, d),7.65 (2H, d) 7.60-7.55 (1H, m),7.45 (1H, dd),3.15 (4H, m), 2.95 (4H, m). | Rₜ 2.27,[M + H]⁺477 |
| 26 | 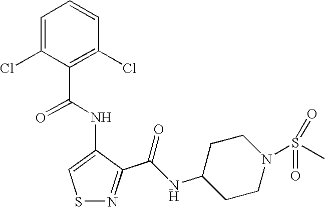 | 4-(2,6-dichloro-benzoylamino)-isothiazole-3-carboxylic acid(1-methanesulfonyl-piperidin-4-yl)-amide | Example 9,using 1-methane-sulphonyl-piperidin-4-yl-amine in step B. | ¹H NMR (d₆-DMSO) 11.20(1H, s), 9.50 (1H,s), 9.15 (1H, d),7.65 (2H, d) 7.60-7.55 (1H, m),3.55 (2H, d),2.87 (3H, s), 2.80(2H, t), 1.83 (2H,dd), 1.70 (2H,qd). | Rₜ 3.12,[M + H]⁺477 |
| 27 | 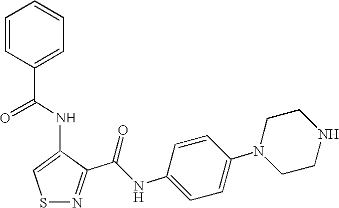 | 4-benzoylamino-isothiazole-3-carboxylic acid(4-piperazin-1-yl-phenyl)-amide | Example 9,using benzoylchloride instep A. | 1H NMR (d₆-DMSO) 11.70(1H, s), 11.00(1H, s), 9.50 (1H,s), 7.95 (2H, d)7.75-7.55 (5H,m), 3.05 (4H, brs), 2.85 (4H, brs). | Rt 3.29,[M + H]+408 |
| 28 | 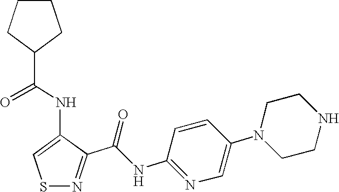 | 4-(cyclopentane-carbonyl-amino)-isothiazole-3-carboxylic acid(5-piperazin-1-yl-pyridin-2-yl)-amide | Example 9,usingcyclopentanecarbonylchloride instep A and 4-(6-amino-pyridin-3-yl)-piperazine-1-carboxylicacid tert-butylester in step B. | 1H NMR (d₆-DMSO) 10.45(1H, s), 10.15(1H, s), 9.15 (1H,s), 8.80 (2H, brs), 8.20 (1H, d)8.00 (1H, d),7.58 (1H, dd),3.45-3.35 (4H,m), 3.25 (4H, brs), 2.90 (1H, p),2.00-1.55 (8H,m) | Rt 2.17,[M + H]+401 |
| 29 | 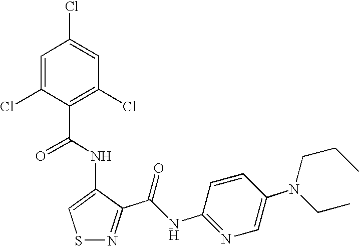 | 4-(2,4,6-trichloro-benzoylamino)-isothiazole-3-carboxylic acid(5-piperazin-1-yl-pyridin-2-yl)-amidetrifluoroaceticacid salt | Example 9,using 2,4,6-trichloro-benzoylchloride instep A and 4-(6-amino-pyridin-3-yl)-piperazine-1-carboxylicacid tert-butylester in step B. | 1H NMR (d₆-DMSO) 10.90(1H, s), 10.30(1H, s), 9.55(1H, s), 8.15 (1H,d) 7.85 (2H, s), 7.55(1H, dd), 3.45-3.35 (4H, m),3.25 (4H, m). | Rt 2.44,[M + H]+511 |
| 30 | 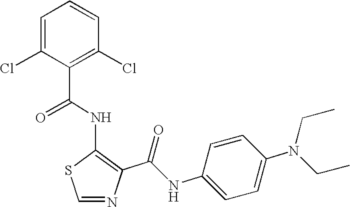 | 5-(2,6-dichloro-benzoylamino)-thiazole-4-carboxylic acid(4-piperazin-1-yl-phenyl)-amide | Example 2,using 2,6-dichloro-benzoylchloride instep B and 4-(4-amino-phenyl)-piperazine-1-carboxylicacid tert-butylester di-hydrochloridein step D. | 1H NMR (d₆-DMSO) 8.50(1H, br s), 8.20(1H, s), 7.55 (2H,d) 7.45 (3H, m),6.90 (2H, d),3.20 (4H, m),3.10 (4H, m). | Rt 2.44,[M + H]+476 |
| 31 | 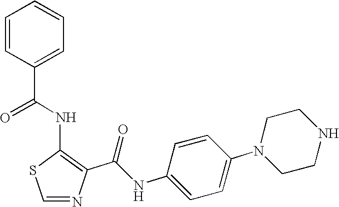 | 5-benzoylamino-thiazole-4-carboxylic acid(4-piperazin-1-yl-phenyl)-amide | Example 2,using benzoylchloride instap B and 4-(4-amino-phenyl)-piperazine-1-carboxylicacid tert-butylester di-hydrochloridein step D. | 1H NMR (d₆-DMSO) 8.70(1H, br s), 8.20(1H, s), 8.00 (2H,d), 7.70 (2H, d)7.65 (3H, m),6.95 (2H, d),3.15 (4H, m),3.00 (4H, m). | Rt 2.22,[M + H]+408 |

### Biological Activity

### Example 32

### Measurement of CDK2 Kinase Inhibitory Activity (IC₅₀)

Compounds of the invention are tested for kinase inhibitory activity using either the following protocol or the activated CDK2/cyclin A kinase protocol described in Example 34.

1.7 μl of active CDK2/CyclinA (Upstate Biotechnology, 10 U/μl) is diluted in assay buffer (250 μl of 10× strength assay buffer (200 mM MOPS pH 7.2, 250 mM β-glycerophosphate, 50 mM EDTA, 150 mM MgCl₂), 11.27 μl 10 mM ATP, 2.5 μl 1M DTT, 25 μl 100 mM sodium orthovanadate, 708.53 μl H₂O), and 10 μl mixed with 10 μl of histone substrate mix (60 μl bovine histone H1 (Upstate Biotechnology, 5 mg/ml), 940 μl H₂O, 35 μCi γ³³P-ATP) and added to 96 well plates along with 5 μl of various dilutions of the test compound in DMSO (up to 2.5%). The reaction is allowed to proceed for 5 hours before being stopped with an excess of ortho-phosphoric acid (30 μl at 2%).

γ³³P-ATP which remains unincorporated into the histone H1 is separated from phosphorylated histone H1 on a Millipore MAPH filter plate. The wells of the MAPH plate are wetted with 0.5% orthophosphoric acid, and then the results of the reaction are filtered with a Millipore vacuum filtration unit through the wells. Following filtration, the residue is washed twice with 200 μl of 0.5% orthophosphoric acid. Once the filters have dried, 25 μl of Microscint 20 scintillant is added, and then counted on a Packard Topcount for 30 seconds.

The % inhibition of the CDK2 activity is calculated and plotted in order to determine the concentration of test compound required to inhibit 50% of the CDK2 activity (IC₅₀).

### Example 33

### CDK Selectivity Assays

Compounds of the invention are tested for kinase inhibitory activity against a number of different kinases using the general protocol described in Example 32, but modified as set out below.

Kinases are diluted to a 10× working stock in 20 mM MOPS pH 7.0, 1 mM EDTA, 0.1% γ-mercaptoethanol, 0.01% Brij-35, 5% glycerol, 1 mg/ml BSA. One unit equals the incorporation of 1 nmol of phosphate per minute into 0.1 mg/ml histone H1, or CDK7 substrate peptide at 30° C. with a final ATP concentration of 100 uM.

The substrate for all the CDK assays (except CDK7) is histone H1, diluted to 10× working stock in 20 mM MOPS pH 7.4 prior to use. The substrate for CDK7 is a specific peptide obtained from Upstate diluted to 10× working stock in deionised water.

Assay Procedure for CDK1/cyclinB, CDK2/cyclinA, CDK2/cyclinE, CDK3/cyclinE, CDK5/p35, CDK6/cyclinD3:

In a final reaction volume of 25 μl, the enzyme (5-10 mU) is incubated with 8 mM MOPS pH 7.0, 0.2 mM EDTA, 0.1 mg/ml histone H1, 10 mM MgAcetate and [γ-³³P-ATP] (specific activity approx 500 cpm/μmol, concentration as required). The reaction is initiated by the addition of Mg²⁺[γ-³³P-ATP]. After incubation for 40 minutes at room temperature the reaction is stopped by the addition of 5 μl of a 3% phosphoric acid solution. 10 ml of the reaction is spotted onto a P30 filter mat and washed 3 times for 5 minutes in 75 mM phosphoric acid and once in methanol prior to drying and counting.

Assay procedure for CDK7/cyclinH/MAT1

In a final reaction volume of 25 μl, the enzyme (5-10 mU) is incubated with 8 mM MOPS pH 7.0, 0.2 mM EDTA, 500 μM peptide, 10 mM MgAcetate and [γ-³³P-ATP] (specific activity approx 500 cpm/μmol, concentration as required). The reaction is initiated by the addition of Mg²⁺[γ-³³P-ATP]. After incubation for 40 minutes at room temperature the reaction is stopped by the addition of 5 μl of a 3% phosphoric acid solution. 10 ml of the reaction is spotted onto a P30 filtermat and washed 3 times for 5 minutes in 75 mM phosphoric acid and once in methanol prior to drying and counting.

### Example 34

### A. Measurement of Activated CDK2/CyclinA Kinase Inhibitory Activity Assay (IC₅₀)

Compounds of the invention are tested for kinase inhibitory activity using the following protocol.

Activated CDK2/CyclinA (Brown et al, Nat. Cell Biol., 1, pp 438-443, 1999; Lowe, E. D., et al Biochemistry, 41, pp 15625-15634, 2002) is diluted to 125 μM in 2.5× strength assay buffer (50 mM MOPS pH 7.2, 62.5 mM β-glycerophosphate, 12.5 mM EDTA, 37.5 mM MgCl₂, 112.5 mM ATP, 2.5 mM DTT, 2.5 mM sodium orthovanadate, 0.25 mg/ml bovine serum albumin), and 10 μl mixed with 10 μl of histone substrate mix (60 μl bovine histone H1 (Upstate Biotechnology, 5 mg/ml), 940 μl H₂O, 35 μCi γ³³P-ATP) and added to 96 well plates along with 5 μl of various dilutions of the test compound in DMSO (up to 2.5%). The reaction is allowed to proceed for 2 to 4 hours before being stopped with an excess of ortho-phosphoric acid (5 μl at 2%).

γ³³P-ATP which remains unincorporated into the histone H1 is separated from phosphorylated histone H1 on a Millipore MAPH filter plate. The wells of the MAPH plate are wetted with 0.5% orthophosphoric acid, and then the results of the reaction are filtered with a Millipore vacuum filtration unit through the wells. Following filtration, the residue is washed twice with 200 μl of 0.5% orthophosphoric acid. Once the filters have dried, 20 μl of Microscint 20 scintillant is added, and then counted on a Packard Topcount for 30 seconds.

The % inhibition of the CDK2 activity is calculated and plotted in order to determine the concentration of test compound required to inhibit 50% of the CDK2 activity (IC₅₀).

Preferred compounds of the formula (I) have IC₅₀ values of less than 20 μM in this assay.

### B. CDK1/CyclinB Assay

CDK1/CyclinB assay is identical to the CDK2/CyclinA above except that CDK1/CyclinB (Upstate Discovery) is used and the enzyme is diluted to 6.25 nM.

### Example 35

### Assay Procedure for CDK4

### Assay A

Assays for CDK4 inhibitory activity are carried out by Proqinase GmbH, Freiburg, Germany using their proprietary 33 PanQinase® Activity Assay. The assays are performed in 96 well FlashPlates™ (PerkinElmer). In each case, the reaction cocktail (50 μl final volume) is composed of; 20 μl assay buffer (final composition 60 mM HEPES-NaOH, pH 7.5, 3 mM MgCl₂, 3 μM Na-orthovanadate, 1.2 mM DTT, 5 [symbol] μg/ml PEG₂₀₀₀, [symbol] μl ATP solution (final concentration 1 μM [γ-³³P]-ATP (approx 5×10⁵ cpm per well)), [symbol] μl test compound (in 10% DMSO), 1 [symbol] μl substrate/1 [symbol] μl enzyme solution (premixed). The final amounts of enzyme and substrate are as below.

| Kinase | Kinase ng/ [symbol] μl | Substrate | Substrate ng/ [symbol] μl |
|---|---|---|---|
| CDK4/CycD1 | 50 | Poly (Ala, Glu, Lys, | 500 |
| | | Tyr) 6:2:5:1 | |

The reaction cocktail is incubated at 30° C. for 80 minutes. The reaction is stopped with 50 μl of 2% H₃PO₄, plates are aspirated and washed twice with 200 μl 0.9% NaCl. Incorporation of ³³P is determined with a microplate scintillation counter. Background values are subtracted from the data before calculating the residual activities for each well. IC₅₀ values are calculated using Prism 3.03.

### Assay B

Compounds of the invention were tested for kinase inhibitory activity using the following protocol.

CDK4/CyclinD1 (Proqinase) is diluted to 12.5 nM in 5 mM Tris pH 7.5, 2.5 mM MgCl2, 25 μM EDTA, 2.5 m M DTT and 125 μM ATP. 10 μl of the enzyme solution is mixed with 10 μl of 100 μl biotin—KAPLSPKKAK₄ (Altabioscience, 1 mM stock −10 mg in 2,250 μl H₂O), 900 μl H₂O, 1 μl 10% triton and 35 μCi γ³³P-ATP) and added to 96 well plates along with 5 μl of various dilutions of the test compound in DMSO (up to 4%). The reaction is allowed to proceed for 2 hours before being stopped with an excess of ortho-phosphoric acid (20 μl at 2%).

γ³³P-ATP which remains unincorporated into the biotin—KAPLSPKKAK4 is separated from phosphorylated biotin—KAPLSPKKAK₄ on a Millipore MAPH filter plate. The wells of the MAPH plate are wetted with 0.5% orthophosphoric acid, and then the results of the reaction are filtered with a Millipore vacuum filtration unit through the wells. Following filtration, the residue is washed twice with 200 μl of 0.5% orthophosphoric acid. Once the filters have dried, 201 of Microscint 20 scintillant is added, and then counted on a Packard Topcount for 30 seconds.

The % inhibition of the CDK4 activity is calculated and plotted in order to determine the concentration of test compound required to inhibit 50% of the CDK4 activity (IC₅₀).

Preferred compounds of the formula (I) have IC₅₀ values of less than 20 μM in this assay.

### Example 36

### Anti-Proliferative Activity

The anti-proliferative activities of compounds of the invention are determined by measuring the ability of the compounds to inhibition of cell growth in a number of cell lines. Inhibition of cell growth is measured using the Alamar Blue assay (Nociari, M. M, Shalev, A., Benias, P., Russo, C. Journal of Immunological Methods 1998, 213, 157-167). The method is based on the ability of viable cells to reduce resazurin to its fluorescent product resorufin. For each proliferation assay cells are plated onto 96 well plates and allowed to recover for 16 hours prior to the addition of inhibitor compounds for a further 72 hours. At the end of the incubation period 10% (v/v) Alamar Blue is added and incubated for a further 6 hours prior to determination of fluorescent product at 535 nM ex/590 nM em. In the case of the non-proliferating cell assay cells are maintained at confluence for 96 hour prior to the addition of inhibitor compounds for a further 72 hours. The number of viable cells is determined by Alamar Blue assay as before. All cell lines are obtained from ECACC (European Collection of cell Cultures).

Preferred compounds of the formula (I) have an IC₅₀ of less than 20 μM when tested against the HCT-116 cell line in this assay.

### Example 37

### Measurement of Inhibitory Activity Against Glycogen Synthase Kinase-3 (GSK-3)

The activities of the compounds of the invention as inhibitors of GSK-3 are determined using either Protocol A or Protocol B below.

### Protocol A

GSK3-β (Upstate Discovery) is diluted to 7.5 n4 in 25 mM MOPS, pH 7.00, 25 mg/ml BSA, 0.0025% Brij-35®, 1.25% glycerol, 0.5 mM EDTA, 25 mM MgCl₂, 0.025% β-mercaptoethanol, 37.5 mM ATP and 10 μl mixed with 10 μl of substrate mix. The substrate mix is 12.5 μM phospho-glycogen synthase peptide-2 (Upstate Discovery) in 1 ml of water with 35 μCi γ³³P-ATP. Enzyme and substrate are added to 96 well plates along with 5 μl of various dilutions of the test compound in DMSO (up to 2.5%). The reaction is allowed to proceed for 3 hours before being stopped with an excess of ortho-phosphoric acid (5 μl at 2%). The filtration procedure is as for Activated CDK2/CyclinA assay above.

### Protocol B

GSK3β (human) is diluted to a 10× working stock in 50 mM Tris pH 7.5, 0.1 mM EGTA, 0.1 mM sodium vanadate, 0.1% β-mercaptoethanol, 1 mg/ml BSA. One unit equals the incorporation of 1 nmol of phosphate per minute phospho-glycogen synthase peptide 2 per minute.

In a final reaction volume of 25 μl, GSK3β (5-10 mU) is incubated with 8 mM MOPS 7.0, 0.2 mM EDTA, 20 μM YRRAAVPPSPSLSRHSSPHQS(P)EDEEE (phospho GS2 peptide), 10 mM MgAcetate and [γ-³³P-ATP] (specific activity approx 500 cpm/pmol, concentration as required). The reaction is initiated by the addition of Mg²+[γ-³³P-ATP]. After incubation for 40 minutes at room temperature the reaction is stopped by the addition of 5 μl of a 3% phosphoric acid solution. 10 μl of the reaction is spotted onto a P30 filter mat and washed 3 times for 5 minutes in 50 mM phosphoric acid and once in methanol prior to drying and counting.

Preferred compounds of the formula (I) have IC₅₀ values of less than 20 μM in this assay.

### Pharmaceutical Formulations

### Example 38

### (i) Tablet Formulation

A tablet composition containing a compound of the formula (I) is prepared by mixing 50 mg of the compound with 197 mg of lactose (BP) as diluent, and 3 mg magnesium stearate as a lubricant and compressing to form a tablet in known manner.

### (ii) Capsule Formulation

A capsule formulation is prepared by mixing 100 mg of a compound of the formula (I) with 100 mg lactose and filling the resulting mixture into standard opaque hard gelatin capsules.

### (iii) Injectable Formulation I

A parenteral composition for administration by injection can be prepared by dissolving a compound of the formula (I) (e.g. in a salt form) in water containing 10% propylene glycol to give a concentration of active compound of 1.5% by weight. The solution is then sterilised by filtration, filled into an ampoule and sealed.

### (iv) Injectable Formulation II

A parenteral composition for injection is prepared by dissolving in water a compound of the formula (I) (e.g. in salt form) (2 mg/ml) and mannitol (50 mg/ml), sterile filtering the solution and filling into sealable 1 ml vials or ampoules.

### (v) Injectable formulation III

A formulation for i.v. delivery by injection or infusion can be prepared by dissolving the compound of formula (I) (e.g. in a salt form) in water at 20 mg/ml. The vial is then sealed and sterilised by autoclaving.

### (vi) Injectable Formulation IV

A formulation for i.v. delivery by injection or infusion can be prepared by dissolving the compound of formula (I) (e.g. in a salt form) in water containing a buffer (e.g. 0.2 M acetate pH 4.6) at 20 mg/ml. The vial is then sealed and sterilised by autoclaving.

### (vii) Subcutaneous Injection Formulation

A composition for sub-cutaneous administration is prepared by mixing a compound of the formula (I) with pharmaceutical grade corn oil to give a concentration of 5 mg/ml. The composition is sterilised and filled into a suitable container.

### (viii) Lyophilised Formulation

Aliquots of formulated compound of formula (I) are put into 50 mL vials and lyophilized. During lyophilisation, the compositions are frozen using a one-step freezing protocol at (−45° C.). The temperature is raised to −10° C. for annealing, then lowered to freezing at 45° C., followed by primary drying at +25° C. for approximately 3400 minutes, followed by a secondary drying with increased steps if temperature to 50° C. The pressure during primary and secondary drying is set at 80 millitor.

### Example 39

### Determination of Antifungal Activity

The antifungal activity of the compounds of the formula (I) is determined using the following protocol.

The compounds are tested against a panel of fungi including Candida parpsilosis, Candida tropicalis, Candida albicans-ATCC 36082 and Cryptococcus neoformans. The test organisms are maintained on Sabourahd Dextrose Agar slants at 4° C. Singlet suspensions of each organism are prepared by growing the yeast overnight at 27° C. on a rotating drum in yeast-nitrogen base broth (YNB) with amino acids (Difco, Detroit, Mich.), pH 7.0 with 0.05 morpholine propanesulphonic acid (MOPS). The suspension is then centrifuged and washed twice with 0.85% NaCl before sonicating the washed cell suspension for 4 seconds (Branson Sonifier, model 350, Danbury, Conn.). The singlet blastospores are counted in a haemocytometer and adjusted to the desired concentration in 0.85% NaCl.

The activity of the test compounds is determined using a modification of a broth microdilution technique. Test compounds are diluted in DMSO to a 1.0 mg/ml ratio then diluted to 64 μg/ml in YNB broth, pH 7.0 with MOPS (Fluconazole is used as the control) to provide a working solution of each compound. Using a 96-well plate, wells 1 and 3 through 12 are prepared with YNB broth, ten fold dilutions of the compound solution are made in wells 2 to 11 (concentration ranges are 64 to 0.125 μg/ml). Well 1 serves as a sterility control and blank for the spectrophotometric assays. Well 12 serves as a growth control. The microtitre plates are inoculated with 10 μl in each of well 2 to 11 (final inoculum size is 10⁴ organisms/ml). Inoculated plates are incubated for 48 hours at 35° C. The MIC values are determined spectrophotometrically by measuring the absorbance at 420 nm (Automatic Microplate Reader, DuPont Instruments, Wilmington, Del.) after agitation of the plates for 2 minutes with a vortex-mixer (Vorte-Genie 2 Mixer, Scientific Industries, Inc., Bolemia, N.Y.). The MIC endpoint is defined as the lowest drug concentration exhibiting approximately 50% (or more) reduction of the growth compared with the control well. With the turbidity assay this is defined as the lowest drug concentration at which turbidity in the well is <50% of the control (IC50). Minimal Cytolytic Concentrations (MCC) are determined by sub-culturing all wells from the 96-well plate onto a Sabourahd Dextrose Agar (SDA) plate, incubating for 1 to 2 days at 35° C. and then checking viability.

### Example 40

### Protocol for the Biological Evaluation of Control of In Vivo Whole Plant Fungal Infection

Compounds of the formula (I) are dissolved in acetone, with subsequent serial dilutions in acetone to obtain a range of desired concentrations. Final treatment volumes are obtained by adding 9 volumes of 0.05% aqueous Tween-20 ™ or 0.01% Triton X-100™, depending upon the pathogen. The compositions are then used to test the activity of the compounds of the invention against tomato blight (Phytophthora infestans) using the following protocol. Tomatoes (cultivar Rutgers) are grown from seed in a soil-less peat-based potting mixture until the seedlings are 10-20 cm tall. The plants are then sprayed to run-off with the test compound at a rate of 100 ppm. After 24 hours the test plants are inoculated by spraying with an aqueous sporangia suspension of Phytophthora infestans, and kept in a dew chamber overnight. The plants are then transferred to the greenhouse until disease develops on the untreated control plants.

Similar protocols are also used to test the activity of the compounds of the invention in combatting Brown Rust of Wheat (Puccinia), Powdery Mildew of Wheat (Ervsiphe vraminis), Wheat (cultivar Monon), Leaf Blotch of Wheat (Septoria tritici), and Glume Blotch of Wheat (Leptosphaeria nodorum).

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1-68. (canceled)

69. A method for the prophylaxis or treatment of a disease state or condition mediated by a cyclin dependent kinase, which method comprises administering to a subject in need thereof a compound of the formula (I): 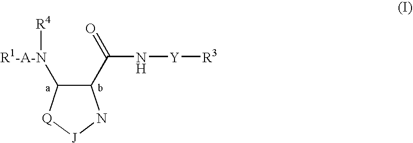
or a salt, tautomer, N-oxide or solvate thereof;
wherein
A is a bond, C═O, NR^{g}(C═O) or O(C═O) wherein R^{g} is hydrogen or C₁₋₄ hydrocarbyl optionally substituted by hydroxy or C₁₋₄ alkoxy;
Y is a bond or an alkylene chain of 1, 2 or 3 carbon atoms in length;
Q is S or CR²;
J is S or CH; provided that one of Q and J is S, and the other of Q and J is not S;
when Q is S, there is a double bond between the ring carbon atoms “a” and “b” and a double bond between the ring nitrogen N and J; and when J is S, there is a double bond between Q and the ring carbon atom “a” and a double bond between the ring nitrogen N and the ring carbon atom “b”;
R¹ is hydrogen; a carbocyclic or heterocyclic group having from 3 to 12 ring members; or a C₁₋₈ hydrocarbyl group optionally substituted by one or more substituents selected from halogen, hydroxy, C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂;
R² is hydrogen; halogen; C₁₋₄ alkoxy; or a C₁₋₄ hydrocarbyl group optionally substituted by halogen, hydroxyl or C₁₋₄ alkoxy;
R³ is selected from hydrogen and carbocyclic and heterocyclic groups having from 3 to 12 ring members; and
R⁴ is hydrogen or a C₁₋₄ hydrocarbyl group optionally substituted by halogen, hydroxyl or C₁₋₄ alkoxy.

70. A method according to claim 69, the compound having the formula (Ib): 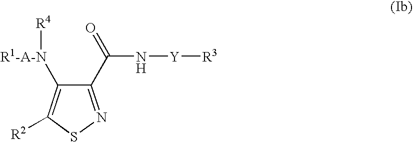
and salts, tautomers, N-oxides or solvates thereof;
wherein
A is a bond, C═O, NR^{g}(C═O) or O(C═O) wherein R^{g} is hydrogen or C₁₋₄ hydrocarbyl optionally substituted by hydroxy or C₁₋₄ alkoxy;
Y is a bond or an alkylene chain of 1, 2 or 3 carbon atoms in length;
R¹ is hydrogen; a carbocyclic or heterocyclic group having from 3 to 12 ring members; or a C₁₋₈ hydrocarbyl group optionally substituted by one or more substituents selected from halogen, hydroxy, C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂;
R² is hydrogen; halogen; C₁₋₄ alkoxy; or a C₁₋₄ hydrocarbyl group optionally substituted by halogen, hydroxyl or C₁₋₄ alkoxy;
R³ is selected from hydrogen and carbocyclic and heterocyclic groups having from 3 to 12 ring members; and
R⁴ is hydrogen or a C₁₋₄ hydrocarbyl group optionally substituted by halogen, hydroxyl or C₁₋₄ alkoxy.

71. A method according to claim 69, the compound having the formula (Id): 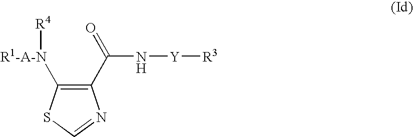
and salts, tautomers, N-oxides or solvates thereof;
wherein
A is a bond, C═O, NR^{g}(C═O) or O(C═O) wherein R^{g} is hydrogen or C₁₋₄ hydrocarbyl optionally substituted by hydroxy or C₁₋₄ alkoxy;
Y is a bond or an alkylene chain of 1, 2 or 3 carbon atoms in length;
R¹ is hydrogen; a carbocyclic or heterocyclic group having from 3 to 12 ring members; or a C₁₋₈ hydrocarbyl group optionally substituted by one or more substituents selected from halogen, hydroxy, C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂;
R³ is selected from hydrogen and carbocyclic and heterocyclic groups having from 3 to 12 ring members; and
R⁴ is hydrogen or a C₁₋₄ hydrocarbyl group optionally substituted by halogen, hydroxyl or C₁₋₄ alkoxy.

72. A compound of the formula (Ia): 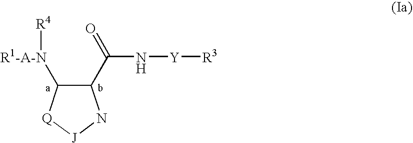
and salts, tautomers, N-oxides and solvates thereof;
wherein
A is a bond, C═O, NR^{g}(C═O) or O(C═O) wherein R^{g} is hydrogen or C₁₋₄ hydrocarbyl optionally substituted by hydroxy or C₁₋₄ alkoxy;
Y is a bond or an alkylene chain of 1, 2 or 3 carbon atoms in length;
Q is S or CR²;
J is S or CH; provided that one of Q and J is S, and the other of Q and J is not S;
when Q is S, there is a double bond between the ring carbon atoms “a” and “b” and a double bond between the ring nitrogen N and J; and when J is S, there is a double bond between Q and the ring carbon atom “a” and a double bond between the ring nitrogen N and the ring carbon atom “b”;
R¹ is (i) a carbocyclic or heterocyclic group having from 3 to 12 ring members; or (ii) a C₁₋₄ hydrocarbyl group substituted by a carbocyclic or heterocyclic group having from 3 to 12 ring members, the hydrocarbyl group being optionally further substituted by one or more substituents selected from halogen, hydroxy, C₁₋₄ hydrocarbyloxy, amino, mono- or di-C₁₋₄ hydrocarbylamino, and wherein 1 or 2 of the carbon atoms of the hydrocarbyl group may optionally be replaced by an atom or group selected from O, S, NH, SO and SO₂;
R² is hydrogen; halogen; C₁₋₄ alkoxy; or a C₁₋₄ hydrocarbyl group optionally substituted by halogen, hydroxy or C₁₋₄ alkoxy;
R³ is selected from carbocyclic and heterocyclic groups having from 3 to 12 ring members; and
R⁴ is hydrogen or a C₁₋₄ hydrocarbyl group optionally substituted by halogen, hydroxy or C₁₋₄ alkoxy;
but excluding:
(A) the compound wherein the ring containing the moiety Q-J is a thiazole ring, A is a bond, R⁴ is hydrogen, R¹ is cyclohexyl, Y is a bond and R³ is a methoxy-substituted dibenzofuran group; and
(B) a compound wherein the ring containing the moiety Q-J is a thiazole ring, A is a bond, R⁴ is hydrogen and R¹ is 4-pyridylmethyl or 5-quinolinyl, and Y—R³ is selected from 3,4-dichlorophenyl, 4-phenoxyphenyl, 4-biphenyl, 4-cyclohexylphenyl and 3-isoquinolinyl.

73. A compound according to claim 72 having the formula (Ic): 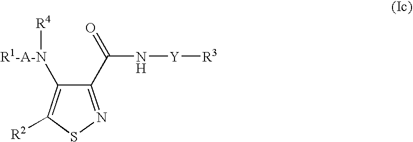
and salts, tautomers, N-oxides and solvates thereof;
wherein
A is a bond, C═O, NR^{g}(C═O) or O(C═O) wherein R^{g} is hydrogen or C₁₋₄ hydrocarbyl optionally substituted by hydroxy or C₁₋₄ alkoxy;
Y is a bond or an alkylene chain of 1, 2 or 3 carbon atoms in length;
R¹ is (i) a carbocyclic or heterocyclic group having from 3 to 12 ring members; or (ii) a C₁₋₄ hydrocarbyl group substituted by a carbocyclic or heterocyclic group having from 3 to 12 ring members, the hydrocarbyl group being optionally further substituted by one or more substituents selected from halogen, hydroxy, C₁₋₄ hydrocarbyloxy, amino, mono- or di-C₁₋₄ hydrocarbylamino, and wherein 1 or 2 of the carbon atoms of the hydrocarbyl group may optionally be replaced by an atom or group selected from O, S, NH, SO and SO₂;
R² is hydrogen; halogen; C₁₋₄ alkoxy; or a C₁₋₄ hydrocarbyl group optionally substituted by halogen, hydroxy or C₁₋₄ alkoxy;
R³ is selected from carbocyclic and heterocyclic groups having from 3 to 12 ring members; and
R⁴ is hydrogen or a C₁₋₄ hydrocarbyl group optionally substituted by halogen, hydroxy or C₁₋₄ alkoxy.

74. A compound according to claim 72 having the formula (Ie): 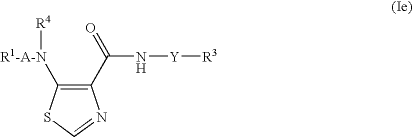
and salts, tautomers, N-oxides and solvates thereof;
wherein
A is a bond, C═O, NR^{g}(C═O) or O(C═O) wherein R^{g} is hydrogen or C₁₋₄ hydrocarbyl optionally substituted by hydroxy or C₁₋₄ alkoxy;
Y is a bond or an alkylene chain of 1, 2 or 3 carbon atoms in length;
R¹ is (i) a carbocyclic or heterocyclic group having from 3 to 12 ring members; or (ii) a C₁₋₄ hydrocarbyl group substituted by a carbocyclic or heterocyclic group having from 3 to 12 ring members, the hydrocarbyl group being optionally further substituted by one or more substituents selected from halogen, hydroxy, C₁₋₄ hydrocarbyloxy, amino, mono- or di-C₁₋₄ hydrocarbylamino, and wherein 1 or 2 of the carbon atoms of the hydrocarbyl group may optionally be replaced by an atom or group selected from O, S, NH, SO and SO₂;
R³ is selected from carbocyclic and heterocyclic groups having from 3 to 12 ring members; and
R⁴ is hydrogen or a C₁₋₄ hydrocarbyl group optionally substituted by halogen, hydroxy or C₁₋₄ alkoxy;
but excluding:
(A) the compound wherein A is a bond, R⁴ is hydrogen, R¹ is cyclohexyl, Y is a bond and R³ is a methoxy-substituted dibenzofuran group; and
(B) a compound wherein A is a bond, R⁴ is hydrogen and R¹ is 4-pyridylmethyl or 5-quinolinyl, and Y—R³ is selected from 3,4-dichlorophenyl, 4-phenoxyphenyl, 4-biphenyl, 4-cyclohexylphenyl and 3-isoquinolyl.

75. A compound according to claim 72 wherein R⁴ is hydrogen.

76. A compound according to claim 72 wherein R^{g} is hydrogen.

77. A compound according to claim 73 wherein R² is hydrogen.

78. A compound according to claim 72 wherein R¹ is (i) a carbocyclic or heterocyclic group having from 3 to 12 ring members; or (ii) a C₁₋₄ hydrocarbyl group substituted by a carbocyclic or heterocyclic group having from 3 to 12 ring members, the hydrocarbyl group being optionally further substituted by one or more substituents selected from halogen, hydroxy, C₁₋₄ hydrocarbyloxy, amino, mono- or di-C₁₋₄ hydrocarbylamino, and wherein 1 or 2 of the carbon atoms of the hydrocarbyl group may optionally be replaced by an atom or group selected from O, S, NH, SO and SO₂.

79. A compound according to claim 78 wherein the carbocyclic or heterocyclic group is an aryl or heteroaryl group, which is optionally substituted by one or more substituent groups R¹⁰ selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; R^{c} is selected from hydrogen and C₁₋₄ hydrocarbyl; and X¹ is O, S or NR^{c} and X² is ═O, ═S or ═NR^{c}.

80. A compound according to claim 79 wherein the substituents are selected from the group R^{10a} consisting of halogen, hydroxy, trifluoromethyl, cyano, nitro, carboxy, a group R^{a}—R^{b} wherein R^{a} is a bond, O, CO, X³C(X⁴), C(X⁴)X³, X³C(X⁴)X³, S, SO, or SO₂, and R^{b} is selected from hydrogen and a C₁₋₈ hydrocarbyl group optionally substituted by one or more substituents selected from hydroxy, oxo, halogen, cyano, nitro, carboxy and monocyclic non-aromatic carbocyclic or heterocyclic groups having from 3 to 6 ring members; wherein one or more carbon atoms of the C₁₋₈ hydrocarbyl group may optionally be replaced by O, S, SO, SO₂, X³C(X⁴), C(X⁴)X³ or X³C(X⁴)X³; X³ is O or S; and X⁴ is ═O or ═S.

81. A compound according to claim 72 having the formula (II): 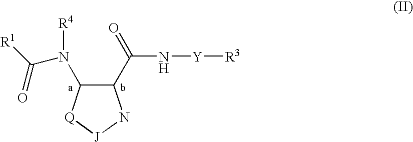
and salts, tautomers, N-oxides and solvates thereof;
wherein Q and J are as defined in claim 72 and R¹, R², R³ and Y are each independently selected from R¹, R², R³ and Y as defined in claim 72.

82. A compound according to claim 81 having the formula (IIa): 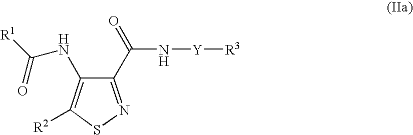
and salts, tautomers, N-oxides and solvates thereof;
wherein R¹, R², R³ and Y are each independently selected from R¹, R², R³ and Y as defined in claim 81.

83. A compound according to claim 81 having the formula (IIb): 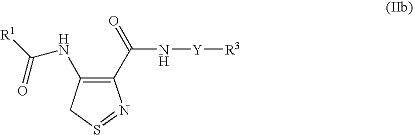
and salts, tautomers, N-oxides and solvates thereof;
wherein R¹, R³ and Y are each independently selected from R¹, R³ and Y as defined in claim 81.

84. A compound according to claim 81 wherein R¹ is:
(i) phenyl optionally substituted by one or more substituents selected from fluorine; chlorine; hydroxy; 5- and 6-membered saturated heterocyclic groups containing 1 or 2 heteroatoms selected from O, N and S, the heterocyclic groups being optionally substituted by one or more C₁₋₄ alkyl groups; C₁₋₄ hydrocarbyloxy; and C₁₋₄ hydrocarbyl; wherein the C₁₋₄ hydrocarbyl and C₁₋₄ hydrocarbyloxy groups are optionally substituted by one or more substituents chosen from hydroxy, fluorine, C₁₋₂ alkoxy, amino, mono and di-C₁₋₄ alkylamino, phenyl, halophenyl, saturated carbocyclic groups having 3 to 7 ring members or saturated heterocyclic groups of 5 or 6 ring members and containing up to 2 heteroatoms selected from O, S and N; or 2,3-dihydro-benzo[1,4]dioxine; or
(ii) a monocyclic heteroaryl group containing one or two heteroatoms selected from O, S and N; or a bicyclic heteroaryl group containing a single heteroatom selected from O, S and N; the monocyclic and bicyclic heteroaryl groups each being optionally substituted by one or more substituents selected from fluorine; chlorine; C₁₋₃ hydrocarbyloxy; and C₁₋₃ hydrocarbyl optionally substituted by hydroxy, fluorine, methoxy or a five or six membered saturated carbocyclic or heterocyclic group containing up to two heteroatoms selected from O, S and N; or
(iii) a substituted or unsubstituted cycloalkyl group having from 3 to 6 ring members; or
(iv) a C₁₋₄ hydrocarbyl group optionally substituted by one or more substituents selected from fluorine; hydroxy; C₁₋₄ hydrocarbyloxy; amino; mono- or di-C₁₋₄ hydrocarbylamino; and carbocyclic or heterocyclic groups having from 3 to 12 ring members, and wherein one of the carbon atoms of the hydrocarbyl group may optionally be replaced by an atom or group selected from O, NH, SO and SO₂.

85. A compound according to claim 84 wherein R¹ is selected from unsubstituted phenyl, 2-fluorophenyl, 2-hydroxyphenyl, 2-methoxyphenyl, 2-methylphenyl, 2-(2-(pyrrolidin-1-yl)ethoxy)-phenyl, 3-fluorophenyl, 3-methoxyphenyl, 2,6-difluorophenyl, 2-fluoro-6-hydroxyphenyl, 2-fluoro-3-methoxyphenyl, 2-fluoro-5-methoxyphenyl, 2-chloro-6-methoxyphenyl, 2-fluoro-6-methoxyphenyl, 2,6-dichlorophenyl, 2-chloro-6-fluorophenyl and 5-fluoro-2-methoxyphenyl.

86. A compound according to claim 85 wherein R¹ is 2,6-dichlorophenyl.

87. A compound according to claim 72 wherein R³ is a non-aromatic group.

88. A compound according to claim 87 wherein R³ is selected from cycloalkyl, oxa-cycloalkyl, aza-cycloalkyl, diaza-cycloalkyl, dioxa-cycloalkyl and aza-oxa-cycloalkyl groups, each unsubstituted or substituted by one or more substituents R¹⁰ or R^{10a} wherein:
R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; R^{c} is selected from hydrogen and C₁₋₄ hydrocarbyl; and X¹ is O, S or NR^{c} and X² is ═O, ═S or ═NR^{c};
R^{10a} is selected from the group consisting of halogen, hydroxy, trifluoromethyl, cyano, nitro, carboxy, a group R^{a}—R^{b} wherein R^{a} is a bond, O, CO, X³C(X⁴), C(X⁴)X³, X³C(X⁴)X³, S, SO, or SO₂, and R^{b} is selected from hydrogen and a C₁₋₈ hydrocarbyl group optionally substituted by one or more substituents selected from hydroxy, oxo, halogen, cyano, nitro, carboxy and monocyclic non-aromatic carbocyclic or heterocyclic groups having from 3 to 6 ring members; wherein one or more carbon atoms of the C₁₋₈ hydrocarbyl group may optionally be replaced by O, S, SO, SO₂, X³C(X⁴), C(X⁴)X³ or X³C(X⁴)X³; X³ is O or S; and X⁴ is ═O or ═S.

89. A compound according to claim 72 having the formula (IV): 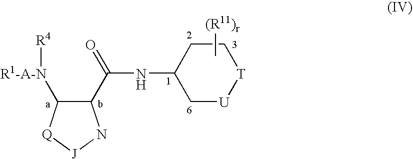
and salts, tautomers, N-oxides and solvates thereof;
wherein A, J, Q, R¹ and R⁴ are as defined in claim 72;
an optional second bond may be present between carbon atoms numbered 1 and 2;
one of U and T is selected from CH₂, CHR¹³, CR¹¹R¹³, NR⁴, N(O)R¹⁵, O and S(O)ₜ; and the other of U and T is selected from, NR¹⁴, O, CH₂, CHR¹¹, C(R¹¹)₂, and C═O; r is 0, 1, 2, 3 or 4; t is 0, 1 or 2;
R¹¹ is selected from hydrogen, halogen (particularly fluorine), C₁₋₃ alkyl and C₁₋₃ alkoxy);
R¹³ is selected from hydrogen, NHR¹⁴, NOH, NOR¹⁴ and R^{a}—R^{b};
R¹⁴ is selected from hydrogen and R^{d}—R^{b};
R^{d} is selected from a bond, CO, C(X²)X¹, SO₂ and SO₂NR^{c};
R^{a}, R^{b} and R^{c} are as hereinbefore defined; and
R¹⁵ is selected from C₁₋₄ saturated hydrocarbyl optionally substituted by hydroxy, C₁₋₂ alkoxy, halogen or a monocyclic 5- or 6-membered carbocyclic or heterocyclic group, provided that U and T cannot be 0 simultaneously.

90. A compound according to claim 89 wherein one of U and T is selected from CH₂, CHR¹³, CR¹¹R¹³, NR⁴, N(O)R¹⁵, O and S(O)ₜ; and the other of U and T is selected from CH₂, CHR¹¹, C(R¹¹)₂, and C═O; r is 0, 1 or 2; t is 0, 1 or 2;
R¹¹ is selected from hydrogen and C₁₋₃ alkyl;
R¹³ is selected from hydrogen and R^{a}—R^{b};
R¹⁴ is selected from hydrogen and R^{d}—R^{b};
R^{d} is selected from a bond, CO, C(X²)X¹, SO₂ and SO₂NR^{c};
R^{a}, R^{b} and R^{c} are as hereinbefore defined; and
R¹⁵ is selected from C₁₋₄ saturated hydrocarbyl optionally substituted by hydroxy, C₁₋₂ alkoxy, halogen or a monocyclic 5- or 6-membered carbocyclic or heterocyclic group.

91. A compound according to claim 90 having the formula (V): 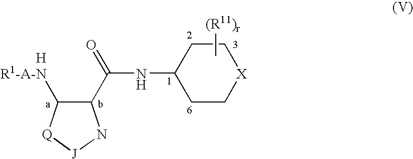
and salts, tautomers, N-oxides and solvates thereof;
wherein Q, J, the ring carbon atoms “a” and “b”, R¹, R¹¹ and “r” are as defined in claim 88 and X is selected from methoxy and a group NR¹⁴ as defined in claim 88.

92. A compound according to claim 90 having the formula (Va): 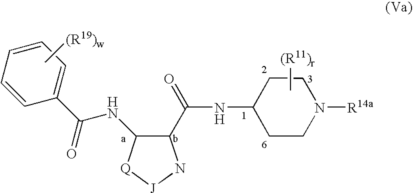
and salts, tautomers, N-oxides and solvates thereof;
wherein R^{14a} is selected from hydrogen, C₁₋₄ alkyl optionally substituted by fluoro, cyclopropylmethyl, phenyl-C₁₋₂ alkyl, C₁₋₄ alkoxycarbonyl, phenyl-C₁₋₂ alkoxycarbonyl, C₁₋₂-alkoxy-C₁₋₂ alkyl, and C₁₋₄ alkylsulphonyl, wherein the phenyl moieties when present are optionally substituted by one to three substituents selected from fluorine, chlorine, C₁₋₄ alkoxy optionally substituted by fluoro or C₁₋₂-alkoxy, and C₁₋₄ alkyl optionally substituted by fluoro or C₁₋₂-alkoxy;
w is 0, 1, 2 or 3;
R² is hydrogen or methyl;
R¹¹ and r are as hereinbefore defined; and
R¹⁹ is selected from fluorine; chlorine; C₁₋₄ alkoxy optionally substituted by fluoro or C₁₋₂-alkoxy; and C₁₋₄ alkyl optionally substituted by fluoro or C₁₋₂-alkoxy.

93. A compound according to claim 92 having the formula (VI): 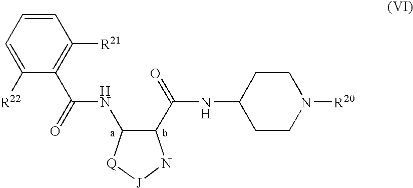
and salts, tautomers, N-oxides and solvates thereof;
wherein R²⁰ is selected from hydrogen and methyl;
R²¹ is selected from fluorine and chlorine; and
R²² is selected from fluorine, chlorine and methoxy; or
one of R²¹ and R²² is hydrogen and the other is selected from chlorine, methoxy, ethoxy, difluoromethoxy, trifluoromethoxy and benzyloxy.

94. A compound according to claim 72 selected from:
4-(2,6-dichloro-benzoylamino)-isothiazole-3-carboxylic acid (1-methyl-piperidin-4-yl)-amide;
5-(2,6-difluoro-benzoylamino)-thiazole-4-carboxylic acid piperidin-4-ylamide;
5-benzylamino-thiazole-4-carboxylic acid piperidin-4-ylamide;
5-(2,6-dichloro-benzylamino)-thiazole-4-carboxylic acid piperidin-4-ylamide;
5-(2-ethoxy-benzoylamino)-thiazole-4-carboxylic acid piperidin-4-ylamide;
5-(1-phenyl-ethylamino)-thiazole-4-carboxylic acid piperidin-4-ylamide;
5-(2-methoxy-benzylamino)-thiazole-4-carboxylic acid piperidin-4-ylamide; 5-(pyridin-3-ylamino)-thiazole-4-carboxylic acid piperidin-4-ylamide;
4-(2,6-dichloro-benzoylamino)-isothiazole-3-carboxylic acid (4-piperazin-1-yl-phenyl)-amide;
5-(2,4,6-trichloro-benzoylamino)-thiazole-4-carboxylic acid phenylamide;
5-(2,4,6-trichloro-benzoylamino)-thiazole-4-carboxylic acid (5-piperazin-1-yl-pyridin-2-yl)-amide;
5-(2,4,6-trichloro-benzoylamino)-thiazole-4-carboxylic acid [1-(tetrahydro-pyran-4-yl)-piperidin-4-yl]-amide;
5-(2,4,6-trichloro-benzoylamino)-thiazole-4-carboxylic acid pyridin-2-yl amide;
5-(2,4,6-trichloro-benzoylamino)-thiazole-4-carboxylic acid piperidin-4-yl amide;
5-(2,4,6-trichloro-benzoylamino)-thiazole-4-carboxylic acid (4-morpholin-4-yl-phenyl)-amide;
5-(2,4,6-trichloro-benzoylamino)-thiazole-4-carboxylic acid (4-methoxy methoxy-cyclohexyl)-amide;
5-(2,4,6-trichloro-benzoylamino)-thiazole-4-carboxylic acid (4-piperazin-1-yl-phenyl)-amide;
5-(2,4,6-trichloro-benzoylamino)-thiazole-4-carboxylic acid (3-hydroxy-phenyl)-amide;
5-(2,4,6-trichloro-benzoylamino)-thiazole-4-carboxylic acid (2-hydroxy-phenyl)-amide;
5-phenylamino-thiazole-4-carboxylic acid phenylamide;
5-phenylamino-thiazole-4-carboxylic acid pyridin-2-yl-amide;
5-phenylamino-thiazole-4-carboxylic acid (5-piperazin-1-yl-pyridin-2-yl)-amide;
5-phenylamino-thiazole-4-carboxylic acid (4-piperazin-1-yl-phenyl)-amide;
5-(2-methoxy-benzylamino)-thiazole-4-carboxylic acid (5-piperazin-1-yl-pyridin-2-yl)-amide;
4-(2,6-dichloro-benzoylamino)-isothiazole-3-carboxylic acid (5-piperazin-1-yl-pyridin-2-yl)-amide;
4-(2,6-dichloro-benzoylamino)-isothiazole-3-carboxylic acid (1-methanesulfonyl-piperidin-4-yl)-amide;
4-benzoylamino-isothiazole-3-carboxylic acid (4-piperazin-1-yl-phenyl)-amide;
4-(cyclopentane-carbonyl-amino)-isothiazole-3-carboxylic acid (5-piperazin-1-yl-pyridin-2-yl)-amide;
4-(2,4,6-trichloro-benzoylamino)-isothiazole-3-carboxylic acid (5-piperazin-1-yl-pyridin-2-yl)-amide;
5-(2,6-dichloro-benzoylamino)-thiazole-4-carboxylic acid (4-piperazin-1-yl-phenyl)-amide; and
5-benzoylamino-thiazole-4-carboxylic acid (4-piperazin-1-yl-phenyl)-amide;
and salts, tautomers, N-oxides and solvates thereof.

95. A composition comprising a pharmaceutically acceptable carrier and a compound of the formula (Ia): 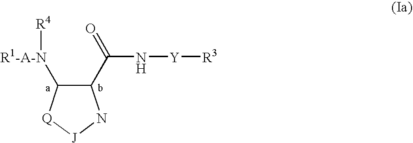
or a salt, tautomer, N-oxide or solvate thereof;
wherein
A is a bond, C═O, NR^{g}(C═O) or O(C═O) wherein R^{g} is hydrogen or C₁₋₄ hydrocarbyl optionally substituted by hydroxy or C₁₋₄ alkoxy;
Y is a bond or an alkylene chain of 1, 2 or 3 carbon atoms in length;
Q is S or CR²;
J is S or CH; provided that one of Q and J is S, and the other of Q and J is not S;
when Q is S, there is a double bond between the ring carbon atoms “a” and “b” and a double bond between the ring nitrogen N and J; and when J is S, there is a double bond between Q and the ring carbon atom “a” and a double bond between the ring nitrogen N and the ring carbon atom “b”;
R¹ is (i) a carbocyclic or heterocyclic group having from 3 to 12 ring members; or (ii) a C₁₋₄ hydrocarbyl group substituted by a carbocyclic or heterocyclic group having from 3 to 12 ring members, the hydrocarbyl group being optionally further substituted by one or more substituents selected from halogen, hydroxy, C₁₋₄ hydrocarbyloxy, amino, mono- or di-C₁₋₄ hydrocarbylamino, and wherein 1 or 2 of the carbon atoms of the hydrocarbyl group may optionally be replaced by an atom or group selected from O, S, NH, SO and SO₂;
R² is hydrogen; halogen; C₁₋₄ alkoxy; or a C₁₋₄ hydrocarbyl group optionally substituted by halogen, hydroxy or C₁₋₄ alkoxy;
R³ is selected from carbocyclic and heterocyclic groups having from 3 to 12 ring members; and
R⁴ is hydrogen or a C₁₋₄ hydrocarbyl group optionally substituted by halogen, hydroxy or C₁₋₄ alkoxy;
but excluding:
(A) the compound wherein the ring containing the moiety Q-J is a thiazole ring, A is a bond, R⁴ is hydrogen, R¹ is cyclohexyl, Y is a bond and R³ is a methoxy-substituted dibenzofuran group; and
(B) a compound wherein the ring containing the moiety Q-J is a thiazole ring, A is a bond, R⁴ is hydrogen and R¹ is 4-pyridylmethyl or 5-quinolinyl, and Y—R³ is selected from 3,4-dichlorophenyl, 4-phenoxyphenyl, 4-biphenyl, 4-cyclohexylphenyl and 3-isoquinolinyl.

96. A method for treating a disease or condition comprising or arising from abnormal cell growth in a mammal, which method comprises administering to the mammal, in an amount effective in inhibiting abnormal cell growth, a compound of the formula (I): 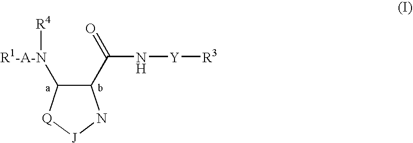
or a salt, tautomer, N-oxide or solvate thereof;
wherein
A is a bond, C═O, NR^{g}(C═O) or O(C═O) wherein R^{g} is hydrogen or C₁₋₄ hydrocarbyl optionally substituted by hydroxy or C₁₋₄ alkoxy;
Y is a bond or an alkylene chain of 1, 2 or 3 carbon atoms in length;
Q is S or CR²;
J is S or CH; provided that one of Q and J is S, and the other of Q and J is not S;
when Q is S, there is a double bond between the ring carbon atoms “a” and “b” and a double bond between the ring nitrogen N and J; and when J is S, there is a double bond between Q and the ring carbon atom “a” and a double bond between the ring nitrogen N and the ring carbon atom “b”;
R¹ is hydrogen; a carbocyclic or heterocyclic group having from 3 to 12 ring members; or a C₁₋₈ hydrocarbyl group optionally substituted by one or more substituents selected from halogen, hydroxy, C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂;
R² is hydrogen; halogen; C₁₋₄ alkoxy; or a C₁₋₄ hydrocarbyl group optionally substituted by halogen, hydroxyl or C₁₋₄ alkoxy;
R³ is selected from hydrogen and carbocyclic and heterocyclic groups having from 3 to 12 ring members; and
R⁴ is hydrogen or a C₁₋₄ hydrocarbyl group optionally substituted by halogen, hydroxyl or C₁₋₄ alkoxy.

97. A method as defined in claim 96 wherein the disease state or condition is selected from proliferative disorders, viral infections, autoimmune diseases and neurodegenerative diseases.

98. A method according to claim 97 wherein the disease state or condition is a cancer selected from breast cancer, ovarian cancer, colon cancer, prostate cancer, oesophageal cancer, squamous cancer, and non-small cell lung carcinomas and hematopoietic tumours of lymphoid lineage, for example leukemia, chronic lymphocytic leukaemia, mantle cell lymphoma and B-cell lymphoma (such as diffuse large B cell lymphoma).

99. A method for the diagnosis and treatment of a disease state or condition mediated by a cyclin dependent kinase, which method comprises (i) screening a patient to determine whether a disease or condition from which the patient is or may be suffering is one which would be susceptible to treatment with a compound having activity against cyclin dependent kinases; and (ii) where it is indicated that the disease or condition from which the patient is thus susceptible, thereafter administering to the patient a compound of the formula (I): 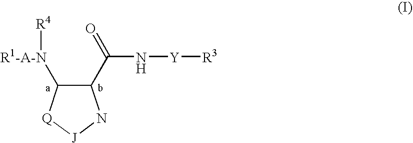
or a salt, tautomer, N-oxide or solvate thereof;
wherein
A is a bond, C═O, NR^{g}(C═O) or O(C═O) wherein R^{g} is hydrogen or C₁₋₄ hydrocarbyl optionally substituted by hydroxy or C₁₋₄ alkoxy;
Y is a bond or an alkylene chain of 1, 2 or 3 carbon atoms in length;
Q is S or CR²;
J is S or CH; provided that one of Q and J is S, and the other of Q and J is not S;
when Q is S, there is a double bond between the ring carbon atoms “a” and “b” and a double bond between the ring nitrogen N and J; and when J is S, there is a double bond between Q and the ring carbon atom “a” and a double bond between the ring nitrogen N and the ring carbon atom “b”;
R¹ is hydrogen; a carbocyclic or heterocyclic group having from 3 to 12 ring members; or a C₁₋₈ hydrocarbyl group optionally substituted by one or more substituents selected from halogen, hydroxy, C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂;
R² is hydrogen; halogen; C₁₋₄ alkoxy; or a C₁₋₄ hydrocarbyl group optionally substituted by halogen, hydroxyl or C₁₋₄ alkoxy;
R³ is selected from hydrogen and carbocyclic and heterocyclic groups having from 3 to 12 ring members; and
R⁴ is hydrogen or a C₁₋₄ hydrocarbyl group optionally substituted by halogen, hydroxyl or C₁₋₄ alkoxy.
